(19) United States
(12) Patent Application Publication
(10) Pub. No.: US 2018/0271581 A1
(43) Pub. Date: Sep. 27, 2018

(54) TISSUE VISUALIZATION AND MODIFICATION DEVICES AND METHODS

(71) Applicant: Trice Medical, Inc., King of Prussia, PA (US)

(72) Inventors: Xiaolong OuYang, Palo Alto, CA (US); James S. Cybulski, Menlo Park, CA (US); Eric Schultz, Menlo Park, CA (US); Fred R. Seddiqui, Los Altos, CA (US)

(21) Appl. No.: 15/921,621

(22) Filed: Mar. 14, 2018

Related U.S. Application Data
(63) Continuation of application No. 14/622,680, filed on Feb. 13, 2015.
Application No. 14/622,680 is a continuation-in-part of application No. 13/447,776, filed on Apr. 16, 2012, now abandoned.
Application No. 13/447,776 is a continuation of application No. 12/269,775, filed on Nov. 12, 2008, now abandoned.
Application No. 14/622,680 is a continuation-in-part of application No. 12/437,865, filed on May 8, 2009, now abandoned.
Application No. 12/437,865 is a continuation-in-part of application No. 12/501,336, filed on Jul. 10, 2009, now abandoned.
Application No. 12/501,336 is a continuation-in-part of application No. 14/526,289, filed on Oct. 28, 2014, now abandoned.
Application No. 14/526,289 is a continuation of application No. 13/085,355, filed on Apr. 12, 2011, now abandoned.
(60) Provisional application No. 61/323,269, filed on Apr. 12, 2010.

(51) Int. Cl. A61B 18/12; A61B 18/14; A61B 1/313; A61B 17/16; A61B 90/30; A61B 90/00

(52) U.S. Cl. CPC A61B 18/1206; A61B 18/1482; A61B 1/3132; A61B 17/1671; A61B 2090/306; A61B 90/361; A61B 2090/309; A61B 17/1606

(57) ABSTRACT

Aspects of the invention include minimally invasive tissue modification systems. Embodiments of the systems include a minimally invasive access device having a proximal end, a distal end and an internal passageway. Positioned among the distal ends of the devices are a visualization element and an illumination element. Also provided are methods of using the systems in tissue modification applications, as well as kits for practicing the methods of the invention. Internal tissue visualization devices having RF-shielded visualization sensor modules are also provided. Minimally invasive RF tissue modulation devices are provided. In some aspects, the devices include a hand-held control unit and an elongated member. In some aspects, RF tissue modulation devices are provided and include an adapter that operably couples to a hand-held medical device. The adapter generates RF energy for delivery to a plasma generator on an elongated member.

### CROSS-REFERENCE TO RELATED APPLICATIONS

The present application is a continuation-in-part of U.S. application Ser. No. 13/447,776 filed on Apr. 16, 2012, entitled MINIMALLY INVASIVE TISSUE MODIFICATION SYSTEMS WITH INTEGRATED VISUALIZATION, which is a continuation of U.S. application Ser. No. 12/269,775, entitled MINIMALLY INVASIVE TISSUE MODIFICATION SYSTEMS WITH INTEGRATED VISUALIZATION, filed on Nov. 12, 2008. The present application is also a continuation-in-part of U.S. application Ser. No. 12/437,865, entitled INTERNAL TISSUE VISUALIZATION SYSTEM COMPRISING A RF-SHIELDED VISUALIZATION SENSOR MODULE, and filed on May 8, 2009. The present application is also a continuation-in-part of U.S. application Ser. No. 12/501,336, entitled HAND-HELD MINIMALLY DIMENSIONED DIAGNOSTIC DEVICE HAVING INTEGRATED DISTAL END VISUALIZATION, and filed on Jul. 10, 2009. The present application is also a continuation-in-part of U.S. application Ser. No. 14/526,289, entitled RF TISSUE MODULATION DEVICES AND METHODS OF USING THE SAME filed on Oct. 28, 2014 which is a continuation of U.S. application Ser. No. 13/085,355, entitled RF TISSUE MODULATION DEVICES AND METHODS OF USING THE SAME, filed on Apr. 12, 2011, which claims the benefit of U.S. Provisional Application No. 61/323,269, entitled RF TISSUE MODULATION DEVICES AND METHODS OF USING THE SAME, and filed on Apr. 12, 2010. The contents of the aforementioned applications are hereby incorporated by reference in their entireties as if fully set forth herein. The benefit of priority to the foregoing applications is claimed under the appropriate legal basis, including, without limitation, under 35 U.S.C. § 119(e).

### INTRODUCTION

Many pathological conditions in the human body may be caused by enlargement, movement, displacement and/or a variety of other changes of bodily tissue, causing the tissue to press against (or “impinge on”) one or more otherwise normal tissues or organs. For example, a cancerous tumor may press against an adjacent organ and adversely affect the functioning and/or the health of that organ. In other cases, bony growths (or “bone spurs”), arthritic changes in bone and/or soft tissue, redundant soft tissue, or other hypertrophic bone or soft tissue conditions may impinge on nearby nerve and/or vascular tissues and compromise functioning of one or more nerves, reduce blood flow through a blood vessel, or both. Other examples of tissues which may grow or move to press against adjacent tissues include ligaments, tendons, cysts, cartilage, scar tissue, blood vessels, adipose tissue, tumor, hematoma, and inflammatory tissue.

The intervertebral disc 10 is composed of a thick outer ring of cartilage (annulus) 12 and an inner gel-like substance (nucleus pulposus) 14. A three-dimensional view of an intervertebral disc 10 is provided in FIG. 1. The annulus 12 contains collagen fibers that form concentric lamellae 16 that surround the nucleus 14 and insert into the endplates of the adjacent vertebral bodies. The nucleus pulposus comprises proteoglycans entrapped by a network of collagen and elastin fibers which has the capacity to bind water. When healthy, the intervertebral disc keeps the spine flexible and serves as a shock absorber by allowing the body to accept and dissipate loads across multiple levels in the spine.

With respect to the spine and intervertebral discs, a variety of medical conditions can occur in which it is desirable to ultimately surgically remove at least some of if not all of an intervertebral disc. As such, a variety of different conditions exist where partial or total disc removal is desirable.

One such condition is disc herniation. Over time, the nucleus pulposus becomes less fluid and more viscous as a result of age, normal wear and tear, and damage caused from an injury. The proteoglycan and water from within the nucleus decreases which in turn results in the nucleus drying out and becoming smaller and compressed. Additionally, the annulus tends to thicken, desiccate, and become more rigid, lessening its ability to elastically deform under load and making it susceptible to disc fissures.

A fissure occurs when the fibrous components of the annulus become separated in particular areas, creating a tear within the annulus. The most common type of fissure is a radial fissure in which the tear is perpendicular to the direction of the fibers. A fissure associated with disc herniation generally falls into three types of categories: 1) contained disc herniation (also known as contained disc protrusion); 2) extruded disc herniation; and 3) sequestered disc herniation (also known as a free fragment.) In a contained herniation, a portion of the disc protrudes or bulges from a normal boundary of the disc but does not breach the outer annulus fibrosis. In an extruded herniation, the annulus is disrupted and a segment of the nucleus protrudes/extrudes from the disc. However, in this condition, the nucleus within the disc remains contiguous with the extruded fragment. With a sequestered disc herniation, a nucleus fragment separates from the nucleus and disc.

As the posterior and posterolateral portions of the annulus are most susceptible to herniation, in many instances, the nucleus pulposus progresses into the fissure from the nucleus in a posteriorly or posterolateral direction. Additionally, biochemicals contained within the nucleus pulposus may escape through the annulus causing inflammation and irritating adjacent nerves. Symptoms of a herniated disc generally include sharp back or neck pain which radiates into the extremities, numbness, muscle weakness, and in late stages, paralysis, muscle atrophy and bladder and bowel incontinence.

Conservative therapy is the first line of treating a herniated disc which includes bed rest, medications to reduce inflammation and pain, physical therapy, patient education on proper body mechanics and weight control.

If conservative therapy offers no improvement then surgery is recommended. Open discectomy is the most common surgical treatment for ruptured or herniated discs. The procedure involves an incision in the skin over the spine to remove the herniated disc material so it no longer presses on the nerves and spinal cord. Before the disc material is removed, some of the bone from the affected vertebra may be removed using a laminotomy or laminectomy to allow the surgeon to better see the area. As an alternative to open surgery, minimally invasive techniques have been rapidly replacing open surgery in treating herniated discs. Minimally invasive surgery utilizes small skin incisions, thereby minimizing the damaging effects of large muscle retraction and offering rapid recovery, less post-operative pain and small incisional scars.

Traditional surgical procedures, both therapeutic and diagnostic, for pathologies located within the body can cause significant trauma to the intervening tissues. These procedures often require a long incision, extensive muscle stripping, prolonged retraction of tissues, de nervation and devascularization of tissue. These procedures can require operating room time of several hours and several weeks of post-operative recovery time due to the destruction of tissue during the surgical procedure. In some cases, these invasive procedures lead to permanent scarring and pain that can be more severe than the pain leading to the surgical intervention.

The development of percutaneous procedures has yielded a major improvement in reducing recovery time and post-operative pain because minimal dissection of tissue, such as muscle tissue, is required. For example, minimally invasive surgical techniques are desirable for spinal and neurosurgical applications because of the need for access to locations within the body and the danger of damage to vital intervening tissues. While developments in minimally invasive surgery are steps in the right direction, there remains a need for further development in minimally invasive surgical instruments and methods.

For the practitioner, the field of diagnostic imaging, for example endoscopy, has allowed for the viewing of objects, internal mechanisms and the like with minimal disruption to the subjects necessarily penetrated to view the afore mentioned objects and mechanisms. Such imaging tools have been used in a wide variety of settings for detailed inspection, including but not limited to the use and application in the field of medicine.

Of particular challenge in the case of using imaging, for example, in the medical field, is the vast amount of equipment typically required, the maintenance of such equipment, and the cabling required for connection to other systems. Among the vast array of equipment required to accomplish an imaging application found in the prior art includes monitor systems, lighting systems and power systems. In addition these systems may be permanently or semi-permanently installed in small offices or operation rooms, for example, which require said offices and rooms to be adapted in potentially a less than ideal fashion so as to accommodate the cumbersomeness of the imaging equipment. In addition, this challenge of the needed installation of imaging systems components may require the duplication of such imaging systems in other offices and rooms as required.

Compounding the above mentioned problem is the requirement that many of these imaging system components must utilize a cabling means to function. These cables that transfer electrical, optical and mechanical means, for example, may physically interfere with objects and persons in the room such as a patient. In some cases, cables for light transmission, for example fiber optic cables, that are rather inflexible may break if over-flexed and thus compromise the outcome of the imaging application.

An additional challenge for imaging technology found in the prior art is the use of external monitoring of the imaging that may be located some distance from the practitioner. As is the case, the practitioner would then be required to view the monitoring of the imaging application in one direction while physically introducing or utilizing the imaging means in a different direction, thus potentially compromising the detail and accuracy of the use of the imaging tool.

Another problem with such imaging systems is that they may require external power. This power must be located relatively proximate to the location of the power outlets and the required voltage available. Since various countries do not share a common power adapter means, or the same voltage output, additional adapters must be utilized for functionality of these systems.

Another challenge faced by imaging systems is satisfaction of the goals of sterility and reusability. Imaging systems must be sterile in order to be employed for their intended applications. While sterility can be accomplished by using a device only once, such approaches are wasteful. However, reusing a device poses significant challenges with respect to maintaining sterility.

### SUMMARY

Aspects of the invention include minimally invasive tissue modification systems. Embodiments of the systems include a minimally invasive access device having a proximal end, a distal end and an internal passageway. The distal end of the access device includes an illumination element. Also part of the system is an elongated tissue modification device having a proximal end and a distal end. The tissue modification device is dimensioned to be slidably moved through the internal passageway of the access device. The tissue modification device includes a tissue modifier and a visualization element integrated at the distal end. Also provided are methods of using the systems in tissue modification applications, as well as kits for practicing the methods of the invention. Additionally, Internal tissue visualization devices having RF-shielded visualization sensor modules are provided. Also provided are systems that include the devices, as well as methods of visualizing internal tissue of a subject using the tissue visualization devices and systems. Hand-held minimally dimensioned diagnostic devices having integrated distal end visualization are provided. Also provided are systems that include the devices, as well as methods of using the devices, e.g., to visualize internal tissue of a subject.

Minimally invasive RF tissue modulation devices are provided. Aspects of the devices include a hand-held control unit and an elongated member. The hand-held control unit includes an electrical energy source and the elongated member has a proximal end operably coupled to the hand-held control unit. A distal end of the elongated member includes a plasma generator. The minimally invasive RF tissue modulation device is configured to generate a plasma at the plasma generator for a therapeutic duration.

An adapter is also provided. Aspects of the invention include an adapter having an electrical energy source, voltage converter, charge accumulator, and RF signal generator.

An RF probe is also provided. Aspects of the RF probe include an elongated member configured to operably couple to a hand-held device at a proximal end of the elongated member. Furthermore, the minimally-dimensioned distal end of the elongated member includes a plasma generator.

A hand-held minimally dimensioned device configured to operably couple to an adapter and an RF probe, such as the ones described above, is also provided. Also provided are kits including a set of components selected from a group consisting of a hand-held device, adapter, RF probe, and other types of probes such as a visualization probe, as described above.

Also provided are methods of delivering the RF energy to the internal target tissue site are also provided. The methods include positioning the distal end of an elongated member of a device, such as the minimally invasive RF tissue modulation device described above, at the internal target tissue site of a subject. The methods also include generating a plasma from the plasma generator to deliver RF energy to the internal target tissue site of the subject

### BRIEF DESCRIPTION OF THE FIGURES

FIG. 1 provides a three-dimensional view of an intervertebral disc according to one embodiment of the invention.

FIG. 2 provides a view of a rongeur modification device according to one embodiment a system of the invention.

FIG. 3 provides views of an access device of a system of the invention configured to be employed with the rongeur modification device according to FIG. 2.

FIG. 4 provides views of an access device of a system of the invention in which the access device is made up of a translucent material and includes a reflective outer coating.

FIG. 5 shows a CMOS visualization sub-system that may be incorporated into a tissue modification system according to an embodiment of the invention.

FIGS. 6A and 6B provide two different views of a disposable tissue visualization and modification device according to an embodiment of the invention.

FIG. 7 provides a view of the distal end of a device according to one embodiment of the invention.

FIG. 8 A is a side view of one embodiment of a portable diagnostic tool.

FIG. 8B is a section view of the portable diagnostic tool of FIG. 8A.

FIG. 8C is a perspective view of the portable diagnostic tool of FIG. 8A.

FIG. 8D is an exploded view of the portable diagnostic tool of FIG. 8A.

FIG. 8E is a perspective, exploded view of the portable diagnostic tool of FIG. 8A

FIG. 8F is a close-up, side view of the portable diagnostic tool of FIG. 8A showing a port for introducing material, medicine and implant.

FIG. 8G is a perspective view of the portable diagnostic tool of FIG. 8A, with the top of the device housing removed to show the geared mechanism between a motor and the elongated member for the purpose of rotating the elongated member along its axis relative to the hand-held control unit, and connections for monitor, lighting, camera and motor to a control board, within the distal portion of the hand piece.

FIG. 8H is one embodiment of the elongated member to motor junction of the portable diagnostic tool of FIG. 8G that shows a friction-based drive connection between a motor and the elongated member for the purpose of rotating the elongated member along its axis relative to the hand-held control unit.

FIG. 8I is a perspective view of the control board, electronics, connections, buttons and switching controls of the portable diagnostic tool of FIG. 8D.

FIG. 8J is a side view of the portable diagnostic tool of FIG. 8A that shows a disconnected elongated member portion of the device from the hand-held control unit.

FIG. 8K is a side view of the portable diagnostic tool of FIG. 8A that shows a disconnected catheter portion of the device and a disconnected monitor portion of the device from the hand-held control unit.

FIG. 9A is a section view of the distal tip of the elongated member of the portable diagnostic tool of FIG. 8A that shows camera, lighting, prism lens and electrical connection.

FIG. 9B shows an embodiment of an image filter within the distal tip of the catheter of FIG. 9A.

FIG. 9C shows another embodiment of an image filter within the distal tip of the elongated member of FIG. 9A.

FIG. 9D is a section view of the distal tip of the elongated member of the portable diagnostic tool of FIG. 8A that shows camera, lighting, flat cover lens and electrical connection.

FIG. 9E shows an image filter configuration according to one embodiment within the distal tip of the catheter of FIG. 9D.

FIG. 9F shows another image filter configuration according to one embodiment within the distal tip of the catheter of FIG. 9D.

FIG. 10A is a front view of the distal tip of an elongated member of the portable diagnostic tool of FIG. 8A that shows an eccentric arrangement between a camera and an integrated illuminator.

FIG. 10B is a front view of the distal tip of the elongated member of the portable diagnostic tool of FIG. 8A that shows an eccentric arrangement between a camera and integrated illuminator, with an additional arrangement of sensors or ports.

FIG. 10C is a front view of the distal tip of an elongated member of a portable diagnostic tool of the invention that shows a concentric arrangement between a camera and an integrated illuminator.

FIG. 10D is a front view of the distal tip of an elongated member of a portable diagnostic tool of the invention that shows a concentric arrangement between a camera and an integrated illuminator, with an additional arrangement of sensors or ports.

FIG. 10E is a section view of the top view of the portable diagnostic tool of FIG. 8A that shows a wiring diagram for a sensor located at the distal tip of the elongated member and connecting to the control board, according to one embodiment of the invention.

FIG. 10F is a section view of the top view of the portable diagnostic tool of FIG. 8A that shows a conduit diagram for a port located at the distal tip of the elongated member and connecting to the port of FIG. 8F, according to one embodiment.

FIG. 11A is a side view of an embodiment for a sterile sheath for the portable diagnostic tool of FIG. 8A that shows an integral monitor cover, control cover, connection to a detachable elongated member, and sealable opening.

FIG. 11B is a side view of an embodiment for a sterile sheath for the portable diagnostic tool of FIG. 8A that shows an integral control cover, connection to a detachable elongated member, and sealable opening.

FIG. 11C is a side view of the sterile sheath of FIG. 11A surrounding the portable diagnostic tool with detached elongated member of FIG. 8I that shows the integral monitor cover over the monitor of FIG. 8I, and an integral control cover over the controls of FIG. 8I.

FIG. 11D is a side view of the sterile sheath of FIG. 11A conforming to the shape of the portable diagnostic tool of FIG. 8A and the opening of FIG. 11A is sealed.

FIG. 11E is a side view of the sterile sheath of FIG. 11B conforming to the shape of the portable diagnostic tool of FIG. 8J with the monitor removed but with the catheter piece attached as in FIG. 8A, and the opening of FIG. 11B is sealed.

FIG. 11F is a side view of the sterile sheath of FIG. 11B conforming to the shape of the portable diagnostic tool of FIG. 8J with the monitor removed and the monitor mount that is located on the hand piece removed but with the elongated member attached as in FIG. 8A, and the opening of FIG. 11B is sealed.

FIG. 12A shows a view of one embodiment for a flexible elongated member section in a straight orientation relative to the axis of the elongated member of FIG. 8A with a control cable.

FIG. 12B shows a view of one embodiment for a flexible elongated member section in a bent or flexed orientation relative to the axis of the elongated member of FIG. 8A with a control cable.

FIG. 12C shows a view of one embodiment for an elongated member in a bent orientation relative to the axis of the elongated member of FIG. 8A.

FIG. 13A is a section view of the distal tip of the elongated member of FIG. 9D showing low-profile biopsy tool that includes an annular member concentrically located at the distal end of the elongated member, and a cable means for actuating the annular member, according to one embodiment.

FIG. 13B is a side view of the distal tip of the elongated member of FIG. 9D showing low-profile biopsy tool that includes an annular member concentrically located at the distal end of the elongated member, and a cable for actuating the former.

FIG. 14 is a section view of the distal tip of the catheter of FIG. 9D showing a low profile cutter concentrically located to the tip of the elongated member.

FIG. 15 is a perspective view of the distal tip of the catheter of FIG. 10F illustrating one embodiment for a slidably present sensor that is in a working channel within the elongated member and can be deployed and remain in a tissue site after the portable diagnostic device of FIG. 8A is removed.

FIG. 16 is a block diagram showing an embodiment of an electronic control schema for the portable diagnostic device of FIG. 8A.

FIG. 17 is a block functional diagram of a stereoscopic imaging module according to one embodiment of the invention.

FIGS. 18A and 18B illustrate off-set views of that may be obtained with a single visualization sensor (FIG. 18A) or two visualization sensors (FIG. 18 B).

FIG. 19A is a side view of one embodiment of a RF tissue modulation device including a elongated member and hand-held control unit.

FIG. 19B is a perspective view of the RF tissue modulation device of FIG. 19A.

FIG. 20A is a cross sectional side view of the distal end of the elongated member of RF tissue modulation device, according to one embodiment.

FIG. 20B is a cross sectional side view of the distal end of the elongated member of RF tissue modulation device, according to one embodiment.

FIG. 20C is a cross sectional side view of the distal end of the elongated member of RF tissue modulation device, according to one embodiment.

FIG. 20D is a cross sectional side view of the distal end of the elongated member of RF tissue modulation device, according to one embodiment.

FIG. 20E is a cross sectional side view of the distal end of the elongated member of an RF tissue modulation device, according to one embodiment.

FIGS. 21A and 21B are side views of an adapter operably coupled to a medical device, according to two different embodiments.

FIG. 22A is a side view of a medical device separated from an adapter configured to operably couple to the medical device, according to one embodiment.

FIG. 22B is a side view of the separated medical device and adapter of FIG. 21A with a removable section of the medical device removed, according to one embodiment.

FIG. 22C is a side view of the adapter and medical device of FIG. 21A operably coupled, according to one embodiment.

FIG. 23 is a side view of an adapter operably coupled to a medical device, according to one embodiment.

FIG. 24 is a functional block diagram of an RF energy source, according to one embodiment.

FIG. 25 is a functional block diagram of an RF energy source, according to one embodiment.

FIG. 26 is a functional block diagram of an RF energy source, according to one embodiment.

FIG. 27 is a block diagram showing an embodiment of the electrical energy source and voltage converter shown for the RF energy source of FIG. 26.

FIG. 28 is a block diagram showing an embodiment of the charge accumulator shown for the RF energy source of FIG. 26.

FIG. 29 is a block diagram showing an embodiment of a modulation circuit coupled to the charge accumulator shown for the RF energy source of FIG. 26.

FIG. 30 is a block diagram showing an embodiment of an RF signal generator and RF tuner shown for the RF energy source of FIG. 26.

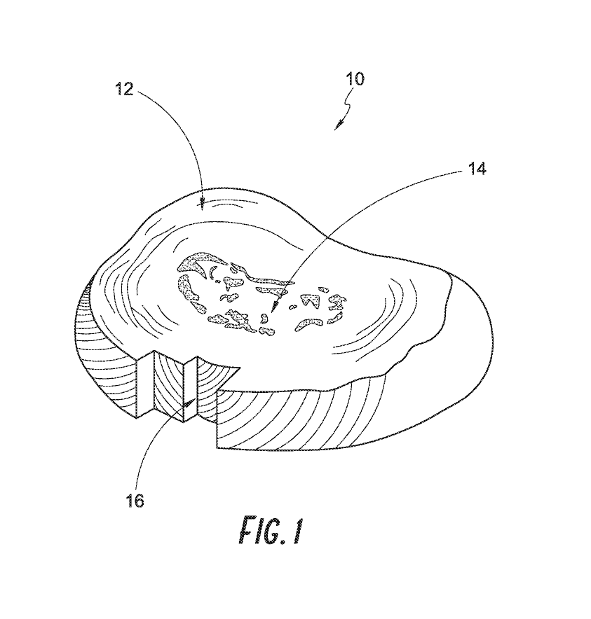

### DETAILED DESCRIPTION

Aspects of the invention include minimally invasive tissue modification systems. Embodiments of the systems include a minimally invasive access device having a proximal end, a distal end and an internal passageway. The distal end of the access device includes an illumination element. Also part of the system is an elongated tissue modification device having a proximal end and a distal end. The tissue modification device is dimensioned to be slidably moved through the internal passageway of the access device. The tissue modification device includes a tissue modifier and a visualization element integrated at the distal end. Also provided are methods of using the systems in tissue modification applications, as well as kits for practicing the methods of the invention. Internal tissue visualization devices having RF-shielded visualization sensor modules are provided. Also provided are systems that include the devices, as well as methods of visualizing internal tissue of a subject using the tissue visualization devices and systems. Hand-held minimally dimensioned diagnostic devices having integrated distal end visualization are provided. Also provided are systems that include the devices, as well as methods of using the devices, e.g., to visualize internal tissue of a subject.

Minimally invasive RF tissue modulation devices are provided. Aspects of the devices include a hand-held control unit and an elongated member. The hand-held control unit includes an electrical energy source and the elongated member has a proximal end operably coupled to the hand-held control unit. A distal end of the elongated member includes a plasma generator. The minimally invasive RF tissue modulation device is configured to generate a plasma at the plasma generator for a therapeutic duration.

An adapter is also provided. Aspects of the invention include an adapter having an electrical energy source, voltage converter, charge accumulator, and RF signal generator.

Also provided are methods of delivering the RF energy to the internal target tissue site are also provided. The methods include positioning the distal end of an elongated member of a device, such as the minimally invasive RF tissue modulation device described above, at the internal target tissue site of a subject. The methods also include generating a plasma from the plasma generator to deliver RF energy to the internal target tissue site of the subject.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention, embodiments of the minimally invasive tissue modification systems and components thereof are reviewed in greater detail, followed by a review of embodiments of methods of using the devices. In further describing various aspects of the invention, aspects of embodiments of the subject tissue visualization devices and systems are described in greater detail. Additionally, embodiments of methods of visualizing an internal target tissue of a subject in which the subject tissue visualization systems may find use are reviewed in greater detail. In further describing various aspects of the invention, aspects of embodiments of the subject RF tissue modulation devices are described first in greater detail. Next, embodiments of methods of modifying, and in some instances additionally visualizing, an internal target tissue of a subject in which the subject RF tissue modulation devices may find use are reviewed in greater detail.

### Minimally Invasive Tissue Modification Systems

As summarized above, aspects of the invention include minimally invasive tissue modification systems. The systems of the invention are minimally invasive, such that they may be introduced to an internal target site of a patient, e.g., a spinal location that is near or inside of an intervertebral disc, through a minimal incision, e.g., an incision that is less than the size of an incision employed for an access device having a outer diameter of 20 mm or larger, e.g., less than 75% the size of such an incision, such as less than 50% of the size of such an incision, or smaller.

Tissue modification systems of the invention include both an access device and an elongated tissue modification device. The access device is a device having a proximal end and a distal end and an internal passageway extending from the proximal end to the distal end. Similarly, the elongated tissue modification device has a proximal end and a distal end and is dimensioned to be slidably moved through the internal passageway of the access device.

Aspects of the invention include a visualization element and an illumination element that are positioned among the distal ends of the access device and the elongated member. The phrase “among the distal ends of the access device and elongated member” means that between the two distal ends, there is positioned at least one visualization element and at least one illumination element. By “located among the distal ends” is meant that the item of interest (e.g., the visualization element, the illumination element) is present at the distal end of the elongate member and/or access device, or near the distal end of the elongate member and/or access device, e.g., within 10 mm or closer to the distal end, such as within 5 mm or closer to the distal end and including within 3 mm or closer to the distal end.

In certain embodiments, the visualization element and illumination are positioned at the distal end of the same member of the system, e.g., at the distal end of the elongated member or at the distal end of the access device. In yet other embodiments, the visualization and illumination elements are present on different components of the device, e.g., where the visualization element is on the elongated member and the illumination element is on the access device, or vice versa. For ease of description, the systems of the invention will now be further described in terms of embodiments where the visualization element is present on the elongated structure and the illumination element is present on the access device.

### Access Devices

Access devices of the invention are elongated elements having an internal passageway that are configured to provide access to a user e.g., a health care professional, such as a surgeon, from an extra-corporeal location to an internal target tissue site, e.g., a location near or in the spine or component thereof, e.g., near or in an intervertebral disc, inside of the disc, etc., through a minimally invasive incision. Access devices of the invention may be cannulas, components of retractor tube systems, etc. As the access devices are elongate, they have a length that is 1.5 times or longer than their width, such as 2 times or longer than their width, including 5 or even 10 times or longer than their width, e.g., 20 times longer than its width, 30 times longer than its width, or longer.

Where the access devices are configured to provide access through a minimally invasive incision, the longest cross-sectional outer dimension of the access devices (for example, the outer diameter of a tube shaped access device, including wall thickness of the access device, which may be a port or cannula in some instances) ranges in certain instances from 5 mm to 50 mm, such as 10 to 20 mm. With respect to the internal passageway, this passageway is dimensioned to provide passage of the tools, e.g., imaging devices, tissue modifiers, etc., from an extra-corporeal site to the internal target tissue location. In certain embodiments, the longest cross-sectional dimension of the internal passageway, e.g., the inner diameter of a tubular shaped access device, ranges in length from 5 to 30 mm, such as 5 to 25 mm, including 5 to 20 mm, e.g., 7 to 18 mm. Where desired, the access devices are sufficiently rigid to maintain mechanical separation of tissue, e.g., muscle, and may be fabricated from any convenient material. Materials of interest from which the access devices may be fabricated include, but are not limited to: metals, such as stainless steel and other medical grade metallic materials, plastics, and the like.

Aspects of the access devices of the invention include the presence of one or more illumination elements that are positioned at the distal end of the access device. By “positioned at the distal end” is meant that the illumination element(s) is present at the distal end of the access device, or near the distal end of the access device, e.g., within 10 mm or closer to the distal end, such as within 5 mm or closer to the distal end and including within 3 mm or closer to the distal end of the access device. A variety of different types of lights sources may be employed as illumination elements, so long as their dimensions are such that they can be positioned at the distal end of the access device. The light sources may be light emitting diodes configured to emit light of the desired wavelength range, or optical conveyance elements, e.g., optical fibers, configured to convey light of the desired wavelength range from a location other than the distal end of the access device, e.g., a location at the proximal end of the access device, to the distal end of the access device. Where desired, the light sources may include a diffusion element to provide for uniform illumination of the target tissue site. Any convenient diffusion element may be employed, including but not limited to a translucent cover or layer (fabricated from any convenient translucent material) through which light from the light source passes and is thus diffused. In certain instances, two or more distinct types of light sources may be present at the distal end, e.g., both LED and fiber optic light sources. The light sources may be integrated with the access device, e.g., may be configured relative to the access device such that the light source is a component of the access device, and cannot be removed from the remainder of the access device without significantly compromising the structure of the access device. As such, the integrated illumination element of these embodiments is not readily removable from the remainder of the access device, such that the illumination element and remainder of the access device form an inter-related whole. The light sources may include a conductive element, e.g., wire, optical fiber, etc., which runs the length of the access device to provide for control of the light source from a location outside the body, e.g., an extracorporeal control device. In certain instances, the access device is fabricated from a translucent material which conducts light from a source apart from the distal end, e.g., from the proximal end, to the distal end. Where desired, a reflective coating may be provided on the outside of the translucent access device to internally reflect light provided from a remote source, e.g., such as an LED at the proximal end, to the distal end of the device. Any convenient reflective coating material may be employed. In those embodiments of the invention where the system includes two or more illumination elements, the illumination elements may emit light of the same wavelength or they may be spectrally distinct light sources, where by “spectrally distinct” is meant that the light sources emit light at wavelengths that do not substantially overlap, such as white light and near-infra-red light, such as the spectrally distinct light sources described in U.S. application Ser. No. 12/269,770 titled “Minimally Invasive Imaging Device” filed on Nov. 12, 2008 the disclosure of which is herein incorporated by reference.

### Tissue Modification Devices

Tissue modification devices of the invention are elongate members having a proximal and distal end, where the elongate members are dimensioned to be slidably moved through the internal passageway of the access device. As this component of the systems is elongate, it has a length that is 1.5 times or longer than its width, such as 2 times or longer than its width, including 5 or even 10 times or longer than its width, e.g., 20 times longer than its width, 30 times longer than its width, or longer. When designed for use in IVD procedures, the elongate member is dimensioned to access an intervertebral disc. By “dimensioned to access an intervertebral disc” is meant that at least the distal end of the device has a longest cross-sectional dimension that is 10 mm or less, such as 8 mm or less and including 7 mm or less, where in certain embodiments the longest cross-sectional dimension has a length ranging from 5 to 10 mm, such as 6 to 9 mm, and including 6 to 8 mm. The elongate member may be solid or include one or more lumens, such that it may be viewed as a catheter. The term “catheter” is employed in its conventional sense to refer to a hollow, flexible or semi-rigid tube configured to be inserted into a body. Catheters of the invention may include a single lumen, or two or more lumens, e.g., three or more lumens, etc., as desired. Depending on the particular embodiment, the elongate members may be flexible or rigid, and may be fabricated from any convenient material.

Where desired, the devices may include a handle or analogous control structure connected to the proximal end of the elongated member and a working element connected to the distal end of the elongated member. The handle, which may include gripping portions or other convenient structures, is operably connected to the tissue modifier at the distal end of the device so that manipulations performed on the handle, for example manually by a surgeon or by a computer controlled actuator, are translated to the tissue modifier to cause the tissue modifier to move in a manner that provides for desired mechanical tissue modification.

The tissue modifier at the distal end may vary considerably. Examples of tissue modifiers that may be present at the distal end include, but are not limited to: mechanical tissue modifiers, such as rongeur forceps, a curette, a scalpel, one or more cutting blades, a scissors, a forceps, a probe, a rasp, a file, an abrasive element, one or more small planes, a rotary powered mechanical shaver, a reciprocating powered mechanical shaver, a powered mechanical burr, etc.; coagulators, electrosurgical electrodes, active agent delivery devices, e.g., needles, etc.

Integrated at the distal end of the tissue modification device, e.g., near to or part of the tissue modification element, is a visualization element. Of interest as visualization elements are imaging sensors. Imaging sensors of interest are miniature in size so as to be integrated with the tissue modification device at the distal end. Miniature imaging sensors of interest are those that, when integrated at the distal end of an elongated structure along with an illumination source, e.g., such as an LED as reviewed below, can be positioned on a probe having a longest cross section dimension of 6 mm or less, such as 5 mm or less, including 4 mm or less, and even 3 mm or less. In certain embodiments, the miniature imaging sensors have a longest cross-section dimension (such as a diagonal dimension) of 5 mm or less, such 3 mm or less, where in certain instances the sensors may have a longest cross-sectional dimension ranging from 2 to 3 mm. In certain embodiments, the miniature imaging sensors have a cross-sectional area that is sufficiently small for its intended use and yet retain a sufficiently high matrix resolution. Certain imaging sensors of the invention have a cross-sectional area (i.e. an x-y dimension, also known as packaged chip size) that is 2 mm×2 mm or less, such as 1.8 mm×1.8 mm or less, and yet have a matrix resolution of 400×400 or greater, such as 640×480 or greater. Imaging sensors of interest are those that include a photosensitive component, e.g., array of photosensitive elements, coupled to an integrated circuit, where the integrated circuit is configured to obtain and integrate the signals from the photosensitive array and output the analog data to a backend processor. The image sensors of interest may be viewed as integrated circuit image sensors, and include complementary metal-oxide-semiconductor (CMOS) sensors and charge-coupled device (CCD) sensors. The image sensors may further include a lens positioned relative to the photosensitive component so as to focus images on the photosensitive component. A signal conductor may be present to connect the image sensor at the distal and to a device at the proximal end of the elongate member, e.g., in the form of one or more wires running along the length of the elongate member from the distal to the proximal end. Imaging sensors of interest include, but are not limited to, those obtainable from: OmniVision Technologies, Inc., Sony Corporations, Cypress Semiconductors, Aptina Imaging. As the imaging sensor(s) is integrated at the distal end of the tissue modification device, it cannot be removed from the remainder of the tissue modification device without significantly compromising the structure of the modification device. As such, the integrated visualization element is not readily removable from the remainder of the tissue modification device, such that the visualization element and remainder of the tissue modification device form an inter-related whole.

While any convenient imaging sensor may be employed in devices of the invention, in certain instances the imaging sensor is a CMOS sensor. Of interest as CMOS sensors are the OmniPixel line of CMOS sensors available from OmniVision (Sunnyvale, Calif.), including the OmniPixel, OmniPixel2, OmniPixel3, OmniPixei3-HS and OmniBSI lines of CMOS sensors. These sensors may be either frontside or backside illumination sensors, and have sufficiently small dimensions while maintained sufficient functionality to be positioned at the distal end of the minimally invasive devices of the invention. Aspects of these sensors are further described in one or more the following U.S. Patents, the disclosures of which are herein incorporated by reference: U.S. Pat. Nos. 7,388,242; 7,368,772; 7,355,228; 7,345,330; 7,344,910; 7,268,335; 7,209,601; 7,196,314; 7,193,198; 7,161,130; and 7,154,137.

In certain embodiments, the systems of the invention are used in conjunction with a controller configured to control illumination of the illumination elements and/or capture of images (e.g., as still imaged or video output) from the image sensors. This controller may take a variety of different formats, including hardware, software and combinations thereof. The controller may be physically located relative to the tissue modification device and/or access device at any convenient location, where the controller may be present at the distal end of the system components, at some point between the distal and proximal ends or at the proximal ends of the system components, as desired. In certain embodiments, the controller may be distinct from the system components, i.e., access device and tissue modification device, such the access device and/or elongated member includes a controller interface for operatively coupling to the distinct controller, or the controller may be integral with the device.

Systems of the invention may include a number of additional components in addition to the tissue modification and access devices as described above. Additional components may include root retractors, device fixation devices, image display units (such as monitors), data processors, e.g., in the form of computers, etc.

The devices or components thereof of the systems may be configured for one time use (i.e., disposable) or be re-usable, e.g., where the components are configured to be used two or more times before disposal, e.g., where the device components are sterilizable.

### Rongeur System Including Integrated Visualization Element

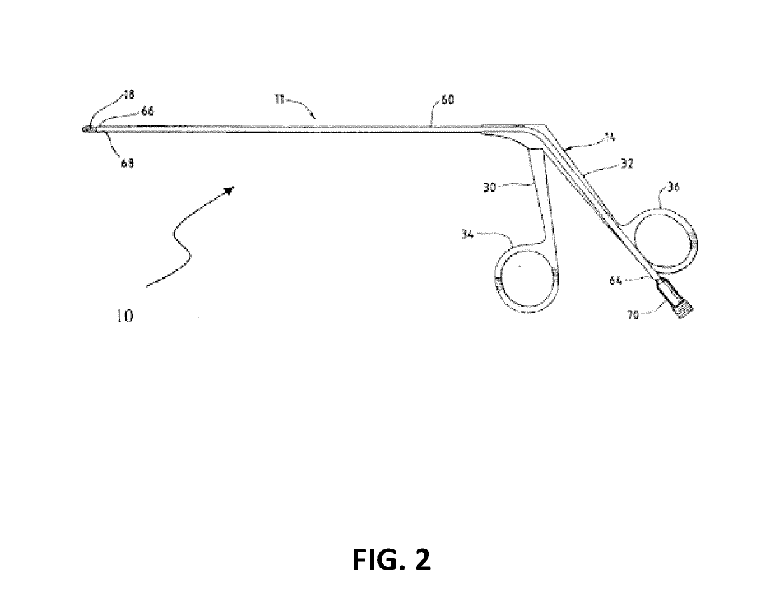

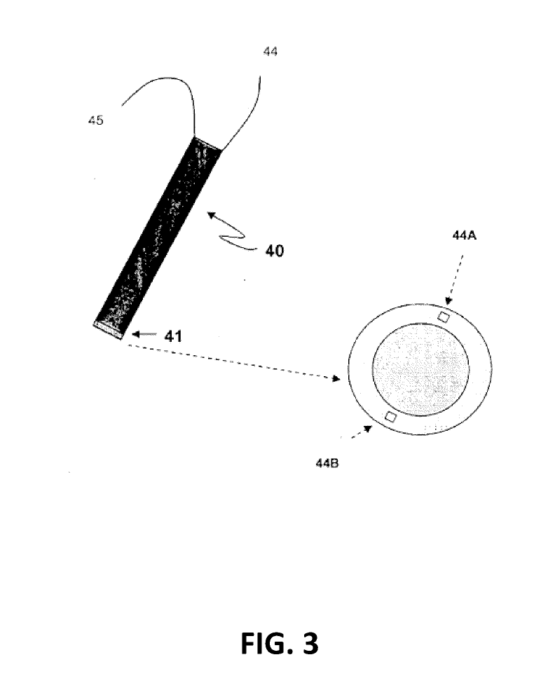

In certain instances, systems of the invention are minimally invasive rongeur systems. The term “rongeur” is employed in its conventional sense to refer to a forceps device configured to remove small pieces of bone or tough tissue. An illustration of a rongeur system according to an embodiment of the invention is depicted in FIGS. 2 and 3.

In FIG. 2, a rongeur device 10 in accordance an embodiment of the present invention is shown. Rongeur device 10 includes elongated member or shaft 11 having a handle 14 mounted on a proximal end 64 of the shaft, and a working element 18 mounted on a distal end 68 of the shaft. The surgical instrument 10 also includes a visualization element, such as a CMOS or CCD camera 66, integrated at the distal end 68 of the device and near to the working element 18. In certain instances, the image sensor may be integrated with the working element itself, such as a forceps member of the working element. The handle 14 has a portion that is intended to be gripped or held by a surgeon so that the working element can be used to manipulate tissue during a surgical procedure.

The handle 14 is offset relative to the shaft 11, and includes a first handle member 30 that is pivotally connected to a second handle member 32. The handle members 30 and 32 terminate in respective finger receiving loops 34 and 36. The handle members 30 and 32 and the loops 34 and 36 form the gripping portion of the handle 14. Also shown at distal end 64 is imaging device interface element 70, which may provide for operative coupling of a wire running the length of the device to monitor (not shown).

The working element 18 is rigidly secured to the distal end 68 of the shaft 11 in any suitable manner. While the working element 18 is in the form of forceps, the working element 18 instead, however, may include a scissors, knife, probe, or coagulator, electrosurgical electrodes, or any other suitable tool.

The shaft 11 may include a central lumen or tube with its proximal end fitted with an interface element 70 in the second handle member 32 (see, e.g., FIG. 2), which interface element 70 allows for operable connection of the integrated visualization element with an external image display unit. The shaft 11 may be straight or have a predetermined bend or curve along its axis. The shaft 11 may be rigid. It may be flexible, bendable or malleable so that it may be adjusted by the surgeon. For example, the shaft may have a distal portion that is displaceable to alternative positions wherein the distal portion does not have the same axis as a proximal portion of the shaft.

The shaft 11 may also include an actuating mechanism operably coupled to the working element 18 to operate the working element. An actuating rod or cable may be affixed to the upper end of the first handle member 30 and extend through a lumen defined by a tube in shaft 11 to join the movable forceps 18. The shaft 11 may be constructed of a stainless steel or any other suitable material.

With this embodiment, by grasping the handle members 30 and 32 by their respective finger-receiving loops 34 and 36, and by pivoting the first handle member 30 back and forth relative to the stationary second handle member 32, the rod or cable moves reciprocally within the tube to cause the forceps or working element 18 to open and close in a scissors-like action.

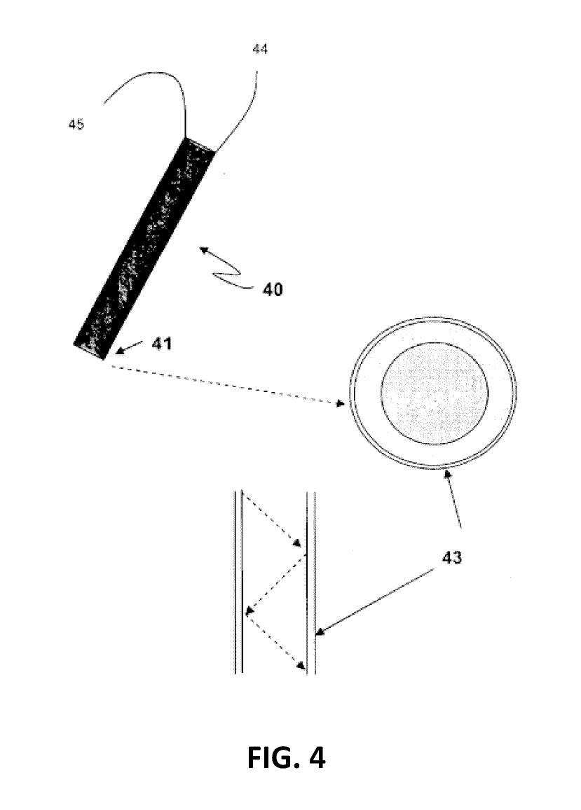

FIG. 3 provides different views of an access device according to an embodiment of the invention. As shown in FIG. 3, access device 40 includes a distal end 41. Positioned at distal end 41 are two illumination sources, e.g., LEDs or light fibers, 44A and 448. Running the length of the access device and exiting the proximal end are wires 44 and 45 for providing power and control to the visualization elements, e.g., via coupling to a control device. FIG. 4 provides a view of an alternative embodiment of the device shown in FIG. 3, where the devices fabricated from a translucent material and includes an outer reflective coating 43 which guides light from the proximal end to the distal end 41. Inner surface of the device also includes a reflective coating to ensure that light can propagate from the proximal end to the distal end of the device.

While the above description with respect to FIGS. 2 and 3 is specifically directed to rongeur systems of the invention, as illustrated above the systems of the invention are not so limited. Instead, systems of the invention include modified versions of any single port laporascopic device system which may include an access device and an instrument configured to be slidably introduced to a tissue location through the access device. Examples of such devices that may be modified to be systems of the invention (for example by including a visualization element on the instrument and an illumination source on the access device) include, but are not limited to: tissue sealers, graspers, dissectors, cautery devices and needle holders, e.g., as sold under the REALHAND™ product line by Novare Surgical Systems, Inc., Cupertino Calif.) and the ENDO AUTONOMY™ LAPARO-ANGLE CHECK product line from Cambridge Endo (Framingham, Mass.).

### Methods

Aspects of the invention further include methods of modifying an internal tissue site with the minimally invasive systems of the invention. A variety of internal tissue sites can be modified with devices of the invention. In certain embodiments, the methods are methods of modifying an intervertebral disc in a minimally invasive manner. For ease of description, the methods are now primarily described further in terms of modifying IVD target tissue sites. However, the invention is not so limited, as the devices may be used to modify a variety of distinct target tissue sites, including those listed above in the introduction section of the present application.

With respect to modifying an intervertebral disc or portion thereof, e.g., herniated portion of a disc, embodiments of such methods include positioning a distal end of a minimally invasive intervertebral disc modification device of the invention in viewing relationship to an intervertebral disc or portion of there, e.g., nucleus pulposus, internal site of nucleus pulposus, etc. By viewing relationship is meant that the distal end is positioned within 40 mm, such as within 10 mm, of the target tissue site of interest. Positioning the distal end in viewing device in relation to the desired target tissue may be accomplished using any convenient approach, including through use of an access device, such as a cannula or retractor tube, which may or may not be fitted with a trocar, as desired, where the access device is a device having illumination element (s) at its distal end. Following positioning of the distal end of the tissue modification device in viewing relationship to the target tissue, the target tissue, e.g., intervertebral disc or portion thereof, is imaged through use of the illumination and visualization elements to obtain image data. Image data obtained according to the methods of the invention is output to a user in the form of an image, e.g., using a monitor or other convenient medium as a display means. In certain embodiments, the image is a still image, while in other embodiments the image may be a video.

Following or during imaging, the methods include a step of tissue modification in addition to the tissue viewing. For example, the methods may include a step of tissue removal, e.g., using forceps of the device to grab and remove target tissue. For example, the methods may include grabbing a least a portion of the herniated tissue of a herniated disc and then removing the grabbed tissue from the site.

Methods of invention may find use in any convenient application, including diagnostic and therapeutic applications. Specific applications of interest include, but are not limited to, intervertebral disc diagnostic and therapeutic applications. For example, methods of the invention include, but are not limited to: annulotomy, nucleotomy, discectomy, annulus replacement, nucleus replacement, and decompression due to a bulging or extruded disc. Additional methods in which the imaging devices find use include those described in United States Published Application Nos. 20080161809; 20080103504; 20080051812; 20080033465; 20070213735; 20070213734; 20070123733; 20070167678; 20070123888; 20060258951; 2006024648; the disclosures of which are herein incorporated by reference.

Methods and devices of the invention may be employed with a variety of subjects. In certain embodiments, the subject is an animal, where in certain embodiments the animal is a “mammal” or “mammalian.” The terms mammal and mammalian are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects (i.e., patients) are humans.

### Embodiments of Kits

Also provided are kits for use in practicing the subject methods, where the kits may include one or more of the above devices, and/or components of the subject systems, as described above. As such, a kit may include a tissue modification device and an access device, as described above. The kit may further include other components, e.g., guidewires, stylets, tissue retractors, etc., which may find use in practicing the subject methods. Various components may be packaged as desired, e.g., together or separately.

In addition to above mentioned components, the subject kits may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Computer Readable Storage Media

Also of interest is programming that is configured for operating a visualization device according to methods of invention, where the programming is recorded on physical computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of instructions for operating a minimally invasive of the invention.

Programming of the invention includes instructions for operating a device of the invention, such that upon execution by the programming, the executed instructions result in execution of the imaging device to: illuminate a target tissue site, such as an intervertebral disc or portion thereof; and capture one or more image frames of the illuminated target tissue site with the imaging sensor.

### Further Embodiments of Tissue Visualization Devices and Systems

As summarized above, aspects of the invention include internal tissue visualization systems. The internal tissue visualization systems are visualization systems that are configured to visualize an internal tissue site of a subject. As such, the systems are structured or designed to provide images of a tissue site inside of a body, such as a living body, to a user. As such, aspects of systems of the invention include internal tissue visualization devices that are useful for visualizing an internal target tissue site, e.g., a spinal location that is near or inside of an intervertebral disc (IVD). The internal tissue visualization devices of embodiments of systems of the invention are dimensioned such that at least the distal end of the devices can pass through a minimally invasive body opening. As such, at least the distal end of the devices of these embodiments may be introduced to an internal target site of a patient, e.g., a spinal location that is near or inside of an intervertebral disc, through a minimal incision, e.g., one that is less than the size of an incision employed for an access device having a outer diameter of 20 mm or smaller, e.g., less than 75% the size of such an incision, such as less than 50% of the size of such an incision, or smaller. In some instances, at least the distal end of the elongated member of the devices is dimensioned to pass through a Cambin's triangle. The Cambin's triangle (also known in the art as the Pambin's triangle) is an anatomical spinal structure bounded by an exiting nerve root and a traversing nerve root and a disc. The exiting root is the root that leaves the spinal canal just cephalad (above) the disc, and the traversing root is the root that leaves the spinal canal just caudad (below) the disc. Where the distal end of the elongated member is dimensioned to pass through a Cambin's triangle, at least the distal end of the device has a longest cross-sectional dimension that is 10 mm or less, such as 8 mm or less and including 7 mm or less. In some instances, the elongated member has an outer diameter that is 7.5 mm or less, such as 7.0 mm or less, including 6.7 mm or less, such as 6.6 mm or less, 6.5 mm or less, 6.0 mm or less, 5.5 mm or less, 5.0 mm or less.

As summarized above, internal tissue visualization devices of the systems of the invention include an elongated member. As this component of the devices is elongated, it has a length that is 1.5 times or longer than its width, such as 2 times or longer than its width, including 5 or even 10 times or longer than its width, e.g., 20 times longer than its width, 30 times longer than its width, or longer. The length of the elongated member may vary, and in some instances ranges from 5 cm to 20 cm, such as 7.5 cm to 15 cm and including 10 to 12 cm. The elongated member may have the same outer cross-sectional dimensions (e.g., diameter) along its entire length. Alternatively, the cross-sectional diameter may vary along the length of the elongated member.

The elongated members of the subject tissue visualization devices have a proximal end and a distal end. The term “proximal end”, as used herein, refers to the end of the elongated member that is nearer the user (such as a physician operating the device in a tissue modification procedure), and the term “distal end”, as used herein, refers to the end of the elongated member that is nearer the internal target tissue of the subject during use. The elongated member is, in some instances, a structure of sufficient rigidity to allow the distal end to be pushed through tissue when sufficient force is applied to the proximal end of the elongate member. As such, in these embodiments the elongated member is not pliant or flexible, at least not to any significant extent.

As summarized above, the visualization devices include a RF-shielded visualization sensor module. The RF-shielded visualization sensor module is integrated with the elongated member. As the RF-shielded visualization sensor module is integrated with the elongated member, it cannot be removed from the remainder of the elongated member and device without significantly compromising the structure and functionality of the device. Accordingly, the devices of the present invention are distinguished from devices which include a “working channel” through which a separate autonomous device is passed through. In contrast to such devices, since the RF-shielded visualization sensor module of the present device is integrated with the elongated member, it is not a separate device from the elongated member that is merely present in a working channel of the elongated member and which can be removed from the working channel of such an elongated member without structurally compromising the elongated member in any way. The visualization sensor module may be integrated with the elongated member by a variety of different configurations. Integrated configurations include configurations where the visualization sensor of the visualization sensor module is fixed relative to the distal end of the elongated member, as well as configurations where the visualization sensor of the visualization sensor module is movable to some extent relative to the distal end of the elongated member. Movement of the visualization sensor of the visualization sensor module may also be provided relative to the distal end of the elongated member, but then fixed with respect to another component present at the distal end, such as a distal end integrated tissue modifier. Specific configurations of interest are further described below in connection with the figures.

As summarized above, devices of the invention include integrated RF-shielded visualization sensor modules. In some instances, a distal end integrated visualization sensor as described herein is present as an RF-shielded visualization module. As the visualization sensor module is RF-shielded, the visualization sensor module includes an RF shield that substantially inhibits, if not completely prevents, an ambient RF field from reaching and interacting with circuitry of the visualization sensor. As such, the RF shield is a structure which substantially inhibits, if not completely prevents, ambient RF energy (e.g., as provided by a distal end RF electrode, as described in greater detail blow) from impacting the circuitry function of the visualization sensor.

Visualization sensor modules of devices of the invention include at least a visualization sensor. In certain embodiments, the devices may further include a conductive member that conductively connects the visualization sensor with another location of the device, such as a proximal end location. Additional components may also be present in the visualization sensor module, where these components are described in greater detail below.

The RF shield of the visualization sensor module may have a variety of different configurations. The RF shield may include an enclosure element or elements which serve to shield the circuitry of the visualization sensor from an ambient RF field. In some instances, the RF shield is a grounded conductive enclosure component or components which are associated with the visualization sensor, conductive member and other components of the visualization sensor module. In some instances, the visualization sensor of the visualization sensor module is present in a housing, where the housing may include a grounder outer conductive layer which serves as an RF shield component. In these instances, the RF shield is an outer grounded conductive layer. The conductive enclosure or enclosures of the RF-shielded visualization sensor module may be fabricated from a variety of different conductive materials, such as metals, metal alloys, etc., where specific conductive materials of interest include, but are not limited to: copper foils and the like. In certain instances, the RF shield is a metallic layer. This layer, when present, may vary in thickness, but in some instances has a thickness ranging from 0.2 mm to 0.7 mm, such as 0.3 mm to 0.6 mm and including 0.4 mm to 0.5 mm.

As reviewed above and elsewhere in the specification, visualization sensor modules of the invention include visualization sensors. Visualization sensors of interest include miniature imaging sensors that have a cross-sectional area which is sufficiently small for its intended use and yet retains a sufficiently high matrix resolution. Imaging sensors of interest are those that include a photosensitive component, e.g., array of photosensitive elements that convert light into electrons, coupled to a circuitry component, such as an integrated circuit. The integrated circuit may be configured to obtain and integrate the signals from the photosensitive array and output image data, which image data may in turn be conveyed to an extra-corporeal device configured to receive the data and display it to a user. The image sensors of these embodiments may be viewed as integrated circuit image sensors. The integrated circuit component of these sensors may include a variety of different types of functionalities, including but not limited to: image signal processing, memory, and data transmission circuitry to transmit data from the visualization sensor to an extra-corporeal location, etc. The miniature imaging sensors may further include a lens component made up of one or more lenses positioned relative to the photosensitive component so as to focus images on the photosensitive component. Specific types of miniature imaging sensors of interest include complementary metal-oxide-semiconductor (CMOS) sensors and charge-coupled device (CCD) sensors. The sensors may have any convenient configuration, including circular, square, rectangular, etc. Visualization sensors of interest may have a longest cross-sectional dimension that varies depending on the particular embodiment, where in some instances the longest cross sectional dimension (e.g., diameter) is 4.0 mm or less, such as 3.5 mm or less, including 3.0 mm or less, such as 2.5 mm or less, including 2.0 mm or less, including 1.5 mm or less, including 1.0 mm or less. Within a given imaging module, the sensor component may be located some distances from the lens or lenses of the module, where this distance may vary, such as 10 mm or less, including 7 mm or less, e.g., 6 mm or less.

Imaging sensors of interest may be either frontside or backside illumination sensors, and have sufficiently small dimensions while maintaining sufficient functionality to be integrated at the distal end of the elongated members of the devices of the invention. Aspects of these sensors are further described in several of the U.S. Patents incorporated by referenced above and herein now, for example: U.S. Pat. Nos. 7,388,242; 7,368,772; 7,355,228; 7,345,330; 7,344,910; 7,268,335; 7,209,601; 7,196,314; 7,193,198; 7,161,130; and 7,154,137.

In some instances, the visualization sensor is located at the distal end of the elongated member, such that the visualization sensor is a distal end visualization sensor. In these instances, the visualization sensor is located at or near the distal end of the elongated member. Accordingly, it is positioned at 3 mm or closer to the distal end, such as at 2 mm or closer to the distal end, including at 1 mm or closer to the distal end. In some instances, the visualization sensor is located at the distal end of the elongated member. The visualization sensor may provide for front viewing and/or side-viewing, as desired. Accordingly, the visualization sensor may be configured to provide image data as seen in the forward direction from the distal end of the elongated member. Alternatively, the visualization sensor may be configured to provide image data as seen from the side of the elongate member. In yet other embodiments, a visualization sensor may be configured to provide image data from both the front and the side, e.g., where the image sensor faces at an angle that is less than 90° relative to the longitudinal axis of the elongated member.

Components of the visualization sensor, e.g., the integrated circuit, one or more lenses, etc., may be present in a housing. The housing may have any convenient configuration, where the particular configuration may be chosen based on location of the sensor, direction of view of the sensor, etc. The housing may be fabricated from any convenient material. In some instances, non-conductive materials, e.g., polymeric materials, are employed.

Visualization sensor modules of devices of the invention may further include functionality for conveying image data to an extra-corporeal device, such as an image display device, of a system. In some instances, a signal cable (or other type of signal conveyance element) may be present to connect the image sensor at the distal end to a device at the proximal end of the elongate member, e.g., in the form of one or more wires running along the length of the elongate member from the distal to the proximal end. In some instances, the visualization sensor is coupled to a conductive member (e.g., cable or analogous structure) that conductively connects the visualization sensor to a proximal end location of the elongated member, where each of these components are present in a conductive enclosure which serves as a RF shield for these components of the visualization sensor module. Alternatively, wireless communication protocols may be employed, e.g., where the imaging sensor is operatively coupled to a wireless data transmitter, which may be positioned at the distal end of the elongated member (including integrated into the visualization sensor, at some position along the elongated member or at the proximal end of the device, e.g., at a location of the proximal end of the elongated member or associated with the handle of the device).

Where desired, the devices may include one or more illumination elements configured to illuminate a target tissue location so that the location can be visualized with a visualization sensor, e.g., as described above. A variety of different types of light sources may be employed as illumination elements (also referred to herein as illuminators), so long as their dimensions are such that they can be positioned at the distal end of the elongated member. The light sources may be integrated with a given component (e.g., elongated member) such that they are configured relative to the component such that the light source element cannot be removed from the remainder of the component without significantly compromising the structure of the component. As such, the integrated illumination element of these embodiments is not readily removable from the remainder of the component, such that the illumination element and remainder of the component form an inter-related whole. The light sources may be light emitting diodes configured to emit light of the desired wavelength range, or optical conveyance elements, e.g., optical fibers, configured to convey light of the desired wavelength range from a location other than the distal end of the elongate member, e.g., a location at the proximal end of the elongate member, to the distal end of the elongate member. The physical location of the light source, e.g., LED, may vary, such as any location in the elongated member, in the hand-held control unit, etc.

As with the image sensors, the light sources may include a conductive element, e.g., wire, or an optical fiber, which runs the length of the elongate member to provide for power and control of the light sources from a location outside the body, e.g., an extracorporeal control device. In some embodiments, the devices are configured such that the RF shielded visualization sensor and the light emitting diode are integrated with the RF-shielded visualization sensor, such that they are coupled to a common RF shielded conductive member that conductively connects the visualization sensor to a proximal end location of the elongated member.

Where desired, the light sources may include a diffusion element to provide for uniform illumination of the target tissue site. Any convenient diffusion element may be employed, including but not limited to a translucent cover or layer (fabricated from any convenient translucent material) through which light from the light source passes and is thus diffused. In those embodiments of the invention where the system includes two or more. illumination elements, the illumination elements may emit light of the same wavelength or they may be spectrally distinct light sources, where by “spectrally distinct” is meant that the light sources emit light at wavelengths that do not substantially overlap, such as white light and infra-red light. In certain embodiments, an illumination configuration as described in copending U.S. application Ser. Nos. 12/269,770 and 12/269,772 (the disclosures of which are herein incorporated by reference) is present in the device.

Depending on the particular device embodiment, the elongated member may or may not include one or more lumens that extend at least partially along its length. When present, the lumens may vary in diameter and may be employed for a variety of different purposes, such as irrigation, aspiration, electrical isolation (for example of conductive members, such as wires), as a mechanical guide, etc., as reviewed in greater detail below. When present, such lumens may have a longest cross section that varies, ranging in some instances from 0.5 to 5.0 mm, such as 1.0 to 4.5 mm, including 1.0 to 4.0 mm. The lumens may have any convenient cross-sectional shape, including but not limited to circular, square, rectangular, triangular, semi-circular, trapezoidal, irregular, etc., as desired. These lumens may be provided for a variety of different functions, including as irrigation and/or aspiration lumens, as described in greater detail below. Such lumens may be employed as a “working channel”.

Where desired, devices of the invention may further include a distal end tissue modifier. Tissue modifiers are components that interact with tissue in some manner to modify the tissue in a desired way. The term modify is used broadly to refer to changing in some way, including cutting the tissue, ablating the tissue, delivering an agent(s) to the tissue, freezing the tissue, etc. As such, of interest as tissue modifiers are tissue cutters, tissue ablators, tissue freezing/heating elements, agent delivery devices, etc. Tissue cutters of interest include, but are not limited to: blades, liquid jet devices, lasers and the like. Tissue ablators of interest include, but are not limited to ablation devices, such as devices for delivery ultrasonic energy (e.g., as employed in ultrasonic ablation), devices for delivering plasma energy, devices for delivering radiofrequency (RF) energy, devices for delivering microwave energy, etc. Energy transfer devices of interest include, but are not limited to: devices for modulating the temperature of tissue, e.g., freezing or heating devices, etc.

In some instances, the tissue modifier includes at least one electrode. For example, tissue modifiers of interest may include RF energy tissue modifiers, which include at least one electrode and may be configured in a variety of different ways depending on the desired configuration of the RF circuit. An RF circuit can be completed substantially entirely at target tissue location of interest (bipolar device) or by use of a second electrode attached to another portion of the patient's body (monopolar device). In either case, a controllable delivery of RF energy is achieved. Aspects of the subject tissue modification devices include a radiofrequency (RF) electrode positioned at the distal end of the elongated member. RF electrodes are devices for the delivery of radiofrequency energy, such as ultrasound, microwaves, and the like. In some instances, the RF electrode is an electrical conductor for delivering RF energy to a particular location, such as a desired target tissue. For instance, in certain cases, the RF electrode can be an RF ablation electrode. RF electrodes of the subject tissue modification devices can include a conductor, such as a metal wire, and can be dimensioned to access an intervertebral disc space. RF electrodes may be shaped in a variety of different formats, such as circular, square, rectangular, oval, etc. The dimensions of such electrodes may vary, where in some embodiments they RF electrode has a longest cross-sectional dimension that is 7 mm or less, 6 mm or less 5 mm or less, 4 mm or less, 3 mm or less or event 2 mm or less, as desired. Where the electrode includes a wire, the diameter of the wire in such embodiments may be 180 μm, such as 150 μm or less, such as 130 μm or less, such as 100 μm or less, such as 80 μm or less. A variety of different RF electrode configurations suitable for use in tissue modification and include, but are not limited to, those described in U.S. Pat. Nos. 7,449,019; 7,137,981; 6,997,941; 6,837,887; 6,241,727; 6,112,123; 6,607,529; 5,334,183. RF electrode systems or components thereof may be adapted for use in devices of the present invention (when coupled with guidance provided by the present specification) and, as such, the disclosures of the RF electrode configurations in these patents are herein incorporated by reference. Specific RF electrode configurations of interest are further described in connection with the figures, below, as well as in U.S. Provisional application Ser. No. 12/422,176; the disclosure of which is herein incorporated by reference.

In some instances, the tissue modifier is integrated at the distal end of the elongated member. In these embodiments, as the tissue modifier is integrated at the distal end of the device, it cannot be entirely removed from the remainder of the device without significantly compromising the structure and functionality of the device. While the tissue modifier cannot entirely be removed from the device without compromising the structure and functionality of the device, components of the tissue modifier may be removable and replaceable. For example, a RF electrode tissue modifier may be configured such that the wire component of the tissue modifier may be replaceable while the remainder of the tissue modifier is not. Accordingly, the devices of the present invention are distinguished from devices which include a “working channel” through which a separate autonomous tissue modifier device, such as an autonomous RF electrode device, is passed through. In contrast to such devices, since the tissue modifier of the present device is integrated at the distal end, it is not a separate device from the elongated member that is merely present in a working channel of the elongated member and which can be removed from the working channel of such an elongated member without structurally compromising the elongated member in any way. The tissue modifier may be integrated with the distal end of the elongated member by a variety of different configurations. Integrated configurations include configurations where the tissue modifier is fixed relative to the distal end of the elongated member, as well as configurations where the tissue modifier is movable to some extent relative to the distal end of the elongated member may be employed in devices of the invention. Specific configurations of interest are further described below in connection with the figures. As the tissue modifier is a distal end integrated tissue modifier, it is located at or near the distal end of the elongated member. Accordingly, it is positioned at 10 mm or closer to the distal end, such as at 5 mm or closer to the distal end, including at 2 mm or closer to the distal end. In some instances, the tissue modifier is located at the distal end of the elongated member.

Depending on the nature of the tissue modifier, the devices will include proximal end connectors for operatively connecting the device and tissue modifier to extracorporeal elements required for operability of the tissue modifier, such as extracorporeal RF controllers (e.g., RF tuners), mechanical tissue cutter controllers, liquid jet controllers, etc.

In some embodiments, an integrated articulation mechanism that imparts steerability to at least one of the visualization sensor, the tissue modifier and the distal end of the elongated member is also present in the device. By “steerability” is meant the ability to maneuver or orient the visualization sensor, tissue modifier and/or distal end of the elongated member as desired during a procedure, e.g., by using controls positioned at the proximal end of the device. In these embodiments, the devices include a steerability mechanism (or one or more elements located at the distal end of the elongated member) which renders the desired distal end component maneuverable as desired through proximal end control. As such, the term “steerability”, as used herein, refers to a mechanism that provides a user steering functionality, such as the ability to change direction in a desired manner, such as by moving left, right, up or down relative to the initial direction. The steering functionality can be provided by a variety of different mechanisms. Examples of suitable mechanisms include, but are not limited to one or more wires, tubes, plates, meshes or combinations thereof, made from appropriate materials, such as shape memory materials, music wire, etc.

In some instances, the distal end of the elongated member is provided with a distinct, additional capability that allows it to be independently rotated about its longitudinal axis when a significant portion of the operating handle is maintained in a fixed position, as discussed in greater detail below. The extent of distal component articulations of the invention may vary, such as from −180 to +1800; e.g., −90 to +900. Alternatively, the distal probe tip articulations may range from 0 to 360°, such as 0 to +1800, and including 0 to +90°, with provisions for rotating the entire probe about its axis so that the full range of angles is accessible on either side of the axis of the probe, e.g., as described in greater detail below. Articulation mechanisms of interest are further described in published PCT Application Publication Nos. WO 2009029639; WO 2008/094444; WO 2008/094439 and WO 2008/094436; the disclosures of which are herein incorporated by reference. Specific articulation configurations of interest are further described in connection with the figures, below, as well as in U.S. application Ser. No. 12/422,176; the disclosure of which is herein incorporated by reference.

In certain embodiments, devices of the invention may further include an irrigator and aspirator configured to flush an internal target tissue site and/or a component of the device, such as a lens of the visualization sensor. As such, the elongated member may further include one or more lumens that run at least the substantial length of the device, e.g., for performing a variety of different functions, as summarized above. In certain embodiments where it is desired to flush (i.e., wash) the target tissue site at the distal end of the elongated member (e.g. to remove ablated tissue from the location, etc.), the elongated member may include both irrigation lumens and aspiration lumens. Thus, the tissue modification device can comprise an irrigation lumen located at the distal end of the elongated member, and the tissue modification device can include an aspiration lumen located at the distal end of the elongated member. During use, the irrigation lumen is operatively connected to a fluid source (e.g., a physiologically acceptable fluid, such as saline) at the proximal end of the device, where the fluid source is configured to introduce fluid into the lumen under positive pressure, e.g., at a pressure ranging from 0 psi to 60 psi, so that fluid is conveyed along the irrigation lumen and out the distal end. While the dimensions of the irrigation lumen may vary, in certain embodiments the longest cross-sectional dimension of the irrigation lumen ranges from 0.5 mm to 5 mm, such as 0.5 mm to 3 mm, including 0.5 mm to 1.5 mm. During use, the aspiration lumen is operatively connected to a source of negative pressure (e.g., a vacuum source) at the proximal end of the device. While the dimensions of the aspiration lumen may vary, in certain embodiments the longest cross-sectional dimension of the aspiration lumen ranges from 1 mm to 7 mm, such as 1 mm to 6 mm, including 1 mm to 5 mm. In some embodiments, the aspirator comprises a port having a cross-sectional area that is 33% or more, such as 50% or more, including 66% or more, of the cross-sectional area of the distal end of the elongated member. In some instances, the negative pressure source is configured to draw fluid and/or tissue from the target tissue site at the distal end into the aspiration lumen under negative pressure, e.g., at a negative pressure ranging from 300 to 600 mmHg, such as 550 mmHg, so that fluid and/or tissue is removed from the tissue site and conveyed along the aspiration lumen and out the proximal end, e.g., into a waste reservoir. In certain embodiments, the irrigation lumen and aspiration lumen may be separate lumens, while in other embodiments, the irrigation lumen and the aspiration lumen can be included in a single lumen, for example as concentric tubes with the inner tube providing for aspiration and the outer tube providing for irrigation. When present, the lumen or lumens of the flushing functionality of the device may be operatively coupled to extra-corporeal irrigation devices, such as a source of fluid, positive and negative pressure, etc. Where desired, irrigators and/or aspirators may be steerable, as described above. Examples of irrigators and aspirators of interest are provided below in greater detail in connection with certain of the figures, as well as in U.S. application Ser. No. 12/422,176; the disclosure of which is herein incorporated by reference.

Where desired, the devices may include a control structure, such as a handle, operably connected to the proximal end of the elongated member. By “operably connected” is meant that one structure is in communication (for example, mechanical, electrical, optical connection, or the like) with another structure. When present, the control structure (e.g., handle) is located at the proximal end of the device. The handle may have any convenient configuration, such as a hand-held wand with one or more control buttons, as a hand-held gun with a trigger, etc., where examples of suitable handle configurations are further provided below.

In some embodiments, the distal end of the elongated member is rotatable about its longitudinal axis when a significant portion of the operating handle is maintained in a fixed position. As such, at least the distal end of the elongated member can turn by some degree while the handle attached to the proximal end of the elongated member stays in a fixed position. The degree of rotation in a given device may vary, and may range from 0 to 360°, such as 0 to 270°, including 0 to 180°.

Devices of the invention may be disposable or reusable. As such, devices of the invention may be entirely reusable (e.g., be multi-use devices) or be entirely disposable (e.g., where all components of the device are single-use). In some instances, the device can be entirely reposable (e.g., where all components can be reused a limited number of times). Each of the components of the device may individually be single-use, of limited reusability, or indefinitely reusable, resulting in an overall device or system comprised of components having differing usability parameters.

Devices of the invention may be fabricated using any convenient materials or combination thereof, including but not limited to: metallic materials such as tungsten, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc.

Systems of the invention further include an extra-corporeal control unit operatively coupled to the proximal end of the elongated member. Extra-corporeal control units may include a number of different components, such as power sources, irrigation sources, aspiration sources, image data processing components, image display components (such as monitors, printers, and the like) for displaying to a user images obtained by the visualization sensor, data processors, e.g., in the form of computers, data storage devices, e.g., floppy disks, hard drives, CD-ROM, DVD, flash memory, etc., device and system controls, etc.

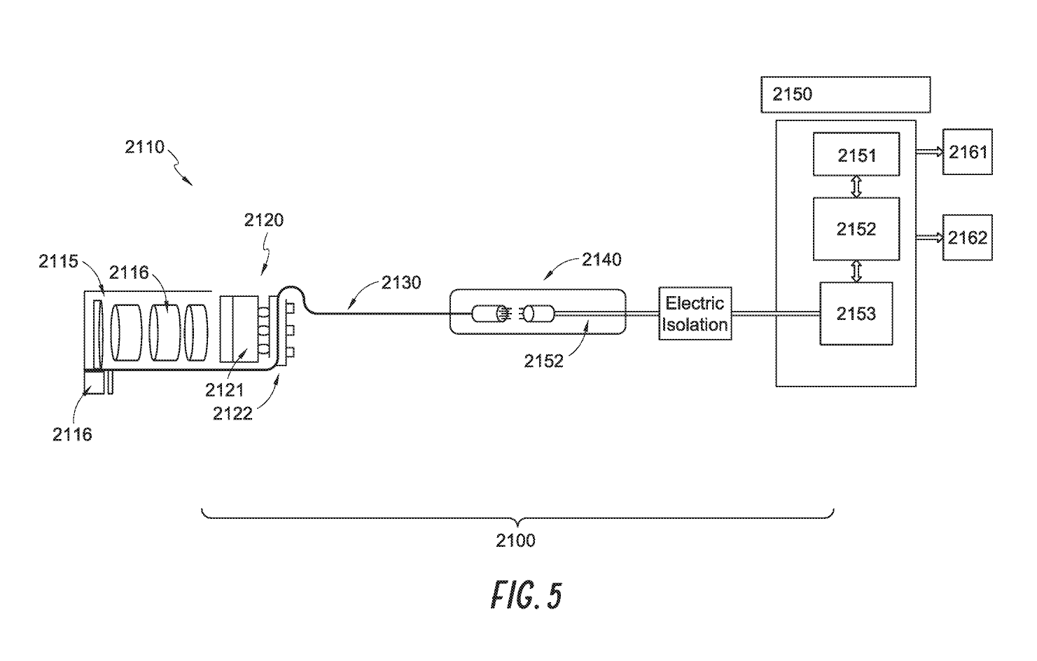

Within a given system, the RF-shielded visualization module may have a variety of different configurations. FIG. 5 provides an example of an embodiment of an integrated RF-shielded visualization module that includes a distal end CMOS visualization sensor and a flexible cable connecting the sensor to the proximal end of the device. As shown in FIG. 5, visualization sensor component 2100 includes distal end CMOS visualization sensor 2110 that includes lens housing 2115 component operatively coupled to integrated circuit component 2120. As shown in the figure, lens housing 2115 includes a lens set 2116. Also shown at the distal end is LED 2118 which provides illumination for a target tissue location during use. Integrated circuit component 2120 includes CMOS sensor integrated circuit 2121 and rigid printed circuit board 2122. The sub-components of lens housing/light source component 2115 are operatively coupled to flexible cable 2130 which provides for operative connection of the CMOS visualization sensor at the distal end of the device via the handle 2140 to the video processing subsystem 2150. In FIG. 5, the entire visualization sensor module (which includes the light source, visualization sensor and flexible cable) is shielded by a conductive outer layer on the visualization sensor housing and a metal tube that surrounds the flexible cable 2130. These enclosures are connected and grounded to provide for RF-shielding of the circuitry components of the visualization sensor. They are also tied to the grounds of the RF circuitry which is associated with the RF electrode of the device (no shown). In the handle 2140 the flexible cable operatively connects to a cable 2152, which cable may have a grounded outer conductive layer that provides for RF isolation. RF shielded cable 2152 connects to video processing sub-system 2150 which includes a variety of functional blocks, such as host controller 2151 (coupled to PC 2161), digital signal processor 2152 (coupled to LCD 2162) and CMOS visualization sensor bridge 2153. As shown in the system of FIG. 5 all the operative components of the visualization sensor, including the integrated circuit, as well as the LED, are operatively coupled to a common printed circuit board, which in turn is coupled to a signal RF shielded cable. This configuration provides numerous advantages in terms of device size, as well as cost and ease of manufacturing.

Systems of the invention may include a number of additional components in addition to the tissue modification devices and extra-corporeal control units, as described above. Additional components may include access port devices; root retractors; retractor devices, system component fixation devices; and the like; etc. Of interest are systems that further access devices as described in co-pending U.S. application Ser. Nos. 12/269,770; 12/269,772; and Ser. No. 12/269,775; the disclosures of which are herein incorporated by reference.

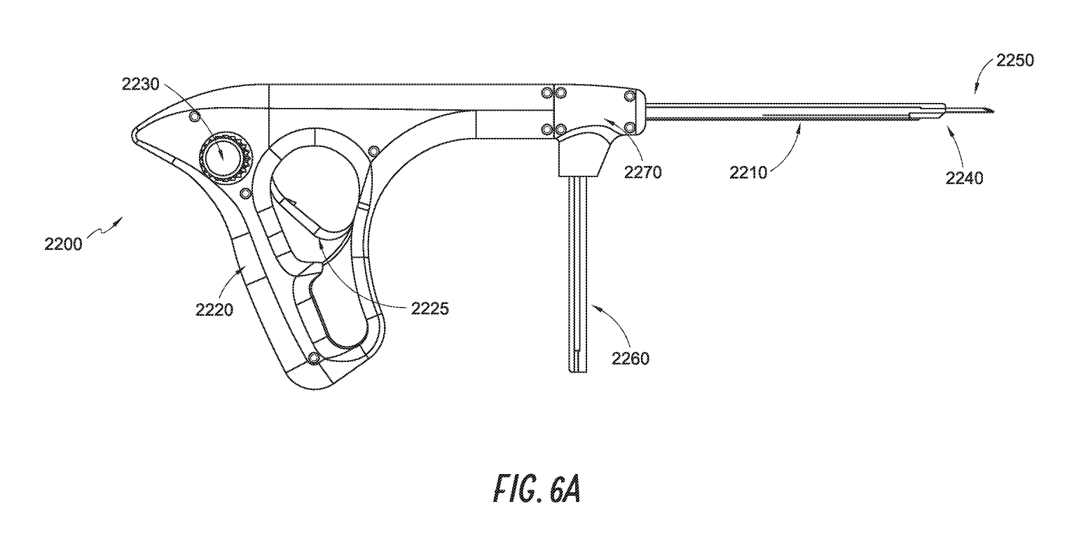

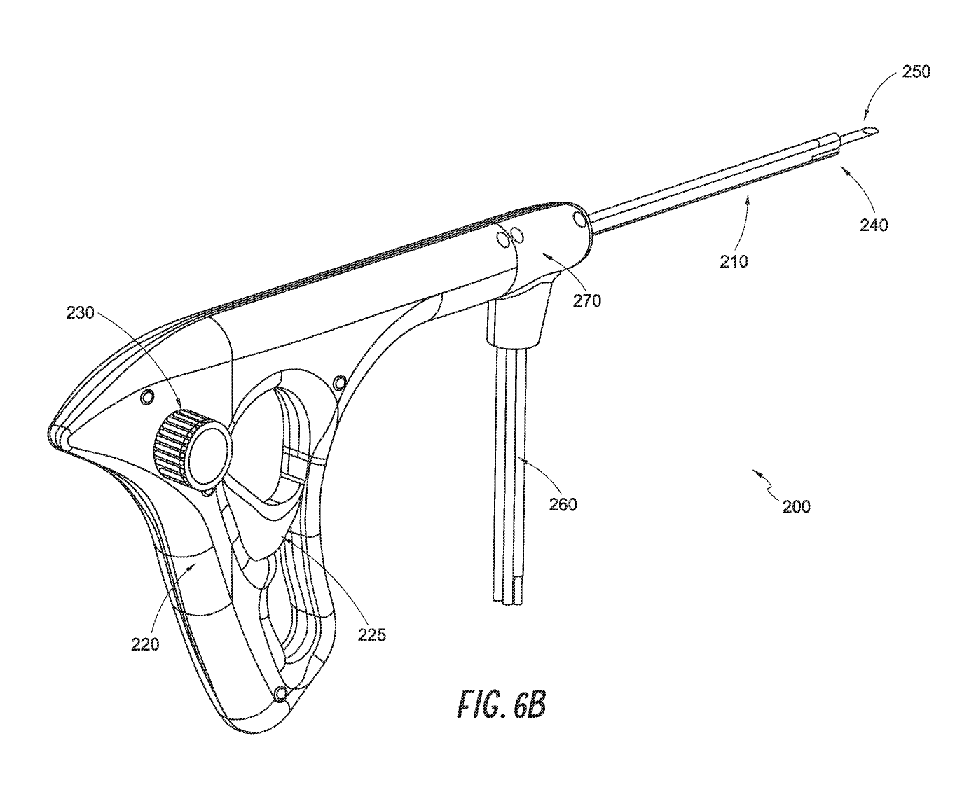

The systems of the invention may include a number of different types of visualization devices. An example of a visualization device is a handheld device as shown in FIGS. 6A and 6B, where the device shown in these figures includes, in addition to the RF shielded distal end integrated visualization sensor, a distal integrated RF electrode tissue modifier and irrigator and aspirator. FIGS. 6A and 6B provide two different side views of a device 2200 according to one embodiment of the invention. Device 2200 includes an elongated member 2210 and an operating handle 2220 at the proximal end of the elongated member 2210. The operating handle has a gun configuration and includes a trigger 2225 and thumbwheel 2230 which provide a user with manual operation over certain functions of the device, e.g., RF electrode positioning and extension. Located at the distal end of the elongated member is an integrated RF-shielded visualization sensor 2240 and tissue modifier 2250. Control elements 2260 (which may include aspiration and irrigation lumens, control/power wires, etc.) exit the handle 2220 at the distal end region 2270, which region 2270 is rotatable relative to the remainder of the handle 2220. A variety of additional components may be present at the distal end of the elongated member, which additional elements may include irrigators, aspirators, articulation mechanisms, etc. as described generally above.

With tissue modification devices of the invention that are configured to be hand-held, e.g., as shown in FIGS. 6A and 6B, the tissue modification devices may have a mass that is 1.5 kg or less, such as 1 kg or less, including 0.5 kg or less, e.g., 0.25 kg or less.

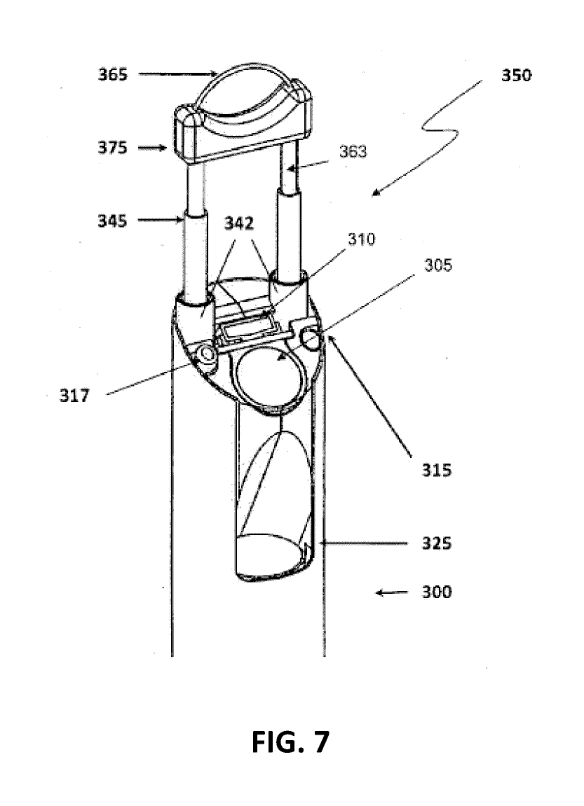

FIG. 7 provides a three-dimensional view of one embodiment of a distal end of tissue visualization device 2300 (having a 6.5 mm outer dimension) of the invention. In FIG. 7, the distal end of the device includes an RF shielded integrated circular CMOS visualization sensor 2305 and integrated LED 2310. Also shown is a first forward facing irrigation lumen 2315 and a second irrigation lumen 2317 which is slightly extended from the distal end and is side facing so that fluid emitted from lumen 2317 is flowed across CMOS visualization sensor 305 to clean the sensor of debris, when needed. Also shown is an aspiration lumen 2325 positioned proximal the irrigation lumens 2315 and 2317 and integrated CMOS visualization sensor 2305, where the aspiration lumen 2325 is configured to aspirate fluid and tissue debris from a target tissue site during use. The distal end further includes an integrated steerable RF electrode assembly 2350. RF electrode assembly 2350 includes NITINOL shape memory guide tubes 2345 extending from insulated (e.g., RF shielded) guide lumens 2342. The RF electrode further includes a tungsten cutting wire 2365 joined at each end to a NITINOL shape memory electrode wire 2363 by a ceramic arc stop 2375. As shown, the diameter of the cutting wire 2365 is smaller than the diameter of the electrode wires 2363, where the difference in size may vary and may range from 100 to 500 μm, such as 300 to 400 μm.

Additional embodiments of tissue modifiers and distal ends of tissue visualization devices of the invention may be found in U.S. application Ser. No. 12/422,176; the disclosure of which is herein incorporated by reference.

### Further Embodiments of Methods of Imaging

Aspects of the subject invention also include methods of imaging (and in some embodiments modifying) an internal target tissue of a subject. Accordingly, aspects of the invention further include methods of imaging an internal tissue site with tissue visualization devices of the invention. A variety of internal tissue sites can be imaged with devices of the invention. In certain embodiments, the methods are methods of imaging an intervertebral disc in a minimally invasive manner. For ease of description, the methods are now primarily described further in terms of imaging IVD target tissue sites. However, the invention is not so limited, as the devices may be used to image a variety of distinct target tissue sites.

With respect to imaging an intervertebral disc or portion thereof, e.g., exterior of the disc, nucleus pulposus, etc., embodiments of such methods include positioning a distal end of a minimally invasive intervertebral disc imaging device of the invention in viewing relationship to an intervertebral disc or portion of there, e.g., nucleus pulposus, internal site of nucleus pulposus, etc. By viewing relationship is meant that the distal end is positioned within 40 mm, such as within 10 mm, including within 5 mm of the target tissue site of interest. Positioning the distal end in viewing device in relation to the desired target tissue may be accomplished using any convenient approach, including through use of an access device, such as a cannula or retractor tube, which may or may not be fitted with a trocar, as desired. Following positioning of the distal end of the imaging device in viewing relationship to the target tissue, the target tissue, e.g., intervertebral disc or portion thereof, is imaged through use of the illumination and visualization elements to obtain image data. Image data obtained according to the methods of the invention is output to a user in the form of an image, e.g., using a monitor or other convenient medium as a display means. In certain embodiments, the image is a still image, while in other embodiments the image may be a video.

In certain embodiments, the methods include a step of tissue modification in addition to the tissue viewing. For example, the methods may include a step of tissue removal, e.g., using a combination of tissue cutting and irrigation or flushing. For example, the methods may include cutting a least a portion of the tissue and then removing the cut tissue from the site, e.g., by flushing at least a portion of the imaged tissue location using a fluid introduced by an irrigation lumen and removed by an aspiration lumen.

The internal target tissue site may vary widely. Internal target tissue sites of interest include, but are not limited to, cardiac locations, vascular locations, orthopedic joints, central nervous system locations, etc. In certain cases, the internal target tissue site comprises spinal tissue.

The subject methods are suitable for use with a variety of mammals. Mammals of interest include, but are not limited to: race animals, e.g. horses, dogs, etc., work animals, e.g. horses, oxen etc., and humans. In some embodiments, the mammals on which the subject methods are practiced are humans.

In some instances, the methods may include obtaining a tissue biopsy with a low-profile biopsy tool. For example, the methods may include advancing an annular cutting member concentrically disposed about the distal end of the elongated member beyond the distal end of the elongated member in a manner sufficient to penetrate and engage target tissue. Following tissue engagement, the annular member may be retracted in the direction of the proximal end of the elongate member in a manner sufficient to secure an amount of tissue with the device which can then be removed from the body to obtain the tissue biopsy.

The subject methods are suitable for use with a variety of mammals. Mammals of interest include, but are not limited to: race animals, e.g. horses, dogs, etc., work animals, e.g. horses, oxen etc., and humans. In some embodiments, the mammals on which the subject methods are practiced are humans.

Aspects of the invention further include methods of assembling an internal tissue visualization device. In these embodiments, the methods include operatively coupling a proximal end of an elongated member to a hand-held control unit, e.g., as described above. Depending on the particular configuration, this step of operatively coupling may include a variety of different actions, such as snapping the elongated member into a receiving structure of the hand-held control unit, twist locking the elongated member into a receiving structure of the hand-held control unit, and the like. In some instances, methods of assembling may further include sealing the hand-held control unit inside of a removable sterile covering, where the sterile covering is attached to the proximal end of the elongated member and configured to seal the hand-held control unit from the environment, e.g., as described above. In such instances, the methods may further include sealing a proximal end of the sterile covering.

### Examples of the Utility of Certain Embodiments

The subject tissue visualization devices and methods find use in a variety of different applications where it is desirable to image and/or modify an internal target tissue of a subject while minimizing damage to the surrounding tissue. The subject devices and methods find use in many applications, such as but not limited to surgical procedures, where a variety of different types of tissues may be removed, including but not limited to: soft tissue, cartilage, bone, ligament, etc. Specific procedures of interest include, but are not limited to, spinal fusion (such as Transforaminal Lumbar Interbody Fusion (TLIF)), total disc replacement (TDR), partial disc replacement (PDR), procedures in which all or part of the nucleus pulposus is removed from the intervertebral disc (IVD) space, arthroplasty, and the like. As such, methods of the invention also include treatment methods, e.g., where a disc is modified in some manner to treat an existing medical condition. Treatment methods of interest include, but are not limited to: annulotomy, nucleotomy, discectomy, annulus replacement, nucleus replacement, and decompression due to a bulging or extruded disc. Additional methods in which the imaging devices find use include those described in United States Published Application No. 20080255563.

In certain embodiments, the subject devices and methods facilitate the dissection of the nucleus pulposus while minimizing thermal damage to the surrounding tissue. In addition, the subject devices and methods can facilitate the surgeon's accessibility to the entire region interior to the outer shell, or annulus, of the IVD, while minimizing the risk of cutting or otherwise causing damage to the annulus or other adjacent structures (such as nerve roots) in the process of dissecting and removing the nucleus pulposus.

Furthermore, the subject devices and methods may find use in other procedures, such as but not limited to ablation procedures, including high-intensity focused ultrasound (HIFU) surgical ablation, cardiac tissue ablation, neoplastic tissue ablation (e.g. carcinoma tissue ablation, sarcoma tissue ablation, etc.), microwave ablation procedures, and the like. Yet additional applications of interest include, but are not limited to: orthopedic applications, e.g., fracture repair, bone remodeling, etc., sports medicine applications, e.g., ligament repair, cartilage removal, etc., neurosurgical applications, and the like.

### Tissue Visualization Devices and Systems

In some instances, at least the distal end region of the elongated member of the devices is dimensioned to pass through a Cambin's triangle. By distal end region is meant a length of the elongated member starting at the distal end of 1 cm or longer, such as 3 cm or longer, including 5 cm or longer, where the elongated member may have the same outer diameter along its entire length. The Cambin's triangle (also known in the art as the Pambin's triangle) is an anatomical spinal structure bounded by an exiting nerve root and a traversing nerve root and a disc. The exiting root is the root that leaves the spinal canal just cephalad (above) the disc, and the traversing root is the root that leaves the spinal canal just caudad (below) the disc. Where the distal end of the elongated member is dimensioned to pass through a Cambin's triangle, at least the distal end of the device has a longest cross-sectional dimension that is 10 mm or less, such as 8 mm or less and including 7 mm or less. In some instances, the devices include an elongated member that has an outer diameter at least in its distal end region that is 5.0 mm or less, such as 4.0 mm or less, including 3.0 mm or less.

As summarized above, the visualization devices include a visualization sensor integrated at the distal end of the elongated member, such that the visualization sensor is integrated with the elongated member. As the visualization sensor is integrated with the elongated member, it cannot be removed from the remainder of the elongated member without significantly compromising the structure and functionality of the elongated member. Accordingly, the devices of the present invention are distinguished from devices which include a “working channel” through which a separate autonomous device is passed through. In contrast to such devices, since the visualization sensor of the present device is integrated with the elongated member, it is not a separate device from the elongated member that is merely present in a working channel of the elongated member and which can be removed from the working channel of such an elongated member without structurally compromising the elongated member in any way. The visualization sensor may be integrated with the elongated member by a variety of different configurations. Integrated configurations include configurations where the visualization sensor is fixed relative to the distal end of the elongated member, as well as configurations where the visualization sensor is movable to some extent relative to the distal end of the elongated member. Movement of the visualization sensor may also be provided relative to the distal end of the elongated member, but then fixed with respect to another component present at the distal end, such as a distal end integrated illuminator. Specific configurations of interest are further described below in connection with the figures.

Distal end integrated illuminators may have any convenient configuration. Configurations of interest have various cross-sectional shapes, including but not limited to circular, ovoid, rectangular (including square), irregular, etc. In some instances the configuration of the integrated illuminator is configured to conform with the configuration of the integrated visualization sensor such that the cross-sectional area of the two components is maximized within the overall minimal cross-sectional area available at the distal end of the elongated member. For example, the configurations of the integrated visualization sensor and illuminators may be such that the integrated visualization sensor may occupy a first portion of the available cross-sectional area of the distal end of the elongated member (such as 40% or more, including 50% or 60% or more of the total available cross-sectional area of the distal end of the elongated member) and the integrated illuminator may occupy a substantial portion of the remainder of the cross-sectional area, such as 60% or more, 70% or more, or 80% or more of the remainder of the cross-sectional area.

In one configuration of interest, the integrated illuminator has a crescent configuration. The crescent configuration may have dimensions configured to confirm with walls of the elongated member, and a circular visualization sensor. In another configuration of interest, the integrated illuminator has an annular configuration, e.g., where conforms to the inner walls of the elongated member or makes up the walls of the elongated member, e.g., as described in greater detail below. This configuration may be of interest where the visualization sensor is positioned at the center of the distal end of the elongated member.

In some instances, the elongated member comprises an annular wall configured to conduct light to the elongated member distal end from a proximal end source. The distal end of this annular wall may be viewed as an integrated illuminator, as described above. In these instances, the walls of the elongated structure which collective make up the annular wall are fabricated from a translucent material which conducts light from a source apart from the distal end, e.g., from the proximal end, to the distal end. Where desired, a reflective coating may be provided on the outside of the translucent elongated member to internally reflect light provided from a remote source, e.g., such as an LED at the proximal end, to the distal end of the device. Any convenient reflective coating material may be employed.

Also of interest are integrated illuminators that include a fluid filled structure that is configured to conduct light to the elongated member distal end from a proximal end source. Such a structure may be a lumen that extends along a length of the elongated structure from a proximal end light source to the distal end of the elongated structure. When present, such lumens may have a longest cross section that varies, ranging in some instances from 0.5 to 4.0 mm, such as 0.5 to 3.5 mm, including 0.5 to 3.0 mm. The lumens may have any convenient cross-sectional shape, including but not limited to circular, square, rectangular, triangular, semi-circular, trapezoidal, irregular, etc., as desired. The fluid filled structure may be filled with any convenient translucent fluid, where fluids of interest include aqueous fluids, e.g., water, saline, etc., organic fluids, such as heavy mineral oil (e.g., mineral oil having a specific gravity greater than or equal to about 0.86 and preferably between about 0.86 and 0.905), and the like.

As indicated above, certain instances of the integrated illuminators are made up of an elongated member integrated light conveyance structure, e.g., optical fiber, light conductive annular wall, light conducting fluid filled structure, etc., which is coupled to a proximal end light source. In some instances, the proximal end light source is a forward focused LED. Of interest are in such embodiments are bright LEDs, e.g., LEDs having a brightness of 100 mcd or more, such as 300 mcd or more, and in some instances 500 mcd or more, 1000 mcd or more, 1500 mcd or more. In some instances, the brightness ranges from 100 to 2000 mcd, such as 300 to 1500 mcd. The LED may be coupled with a forward focusing lens that is, in turn, coupled to the light conveyance structure.

In some instances, the proximal end LED may be coupled to the light conveyance structure in a manner such that substantially all, if not all, light emitted by the LED is input into the light conveyance structure. Alternatively, the LED and focusing lens may be configured such that at least a portion of the light emitted by the LED is directed along the outer surface of the elongated member. In these instances, the forward focused light emitting diode is configured to direct light along the outer surface of the elongated member. As such, light from the proximal end LED travels along the outer surface of the elongated member to the distal end of the elongated member.

In some instances, the tissue visualization devices of the invention or the RF tissue modulation devices described below are configured to reduce coupling of light directly from the integrated illuminator to the visualization sensor. In other words, the devices are structures so that substantially all, if not all, of the light emitted by the integrated illuminator at the distal end of the elongated structure is prevented from directly reaching the visualization sensor. In this manner, the majority, if not all, of the light that reaches the visualization sensor is reflected light, which reflected light is converted to image data by the visualization sensor. In order to substantially prevent, if not inhibit, light from the integrated illuminator from directly reaching the integrated visualization sensor, the device may include a distal end polarized member. By distal end polarized member is meant a structure or combination of structures that have been polarized in some manner sufficient to achieve the desired purpose of reducing, if not eliminating, light from the integrated illuminator directly reaching the integrated visualization sensor. In one embodiment, the light from an LED is polarized by a first polarizer (linearly or circularly) as it enters at lens or prism at the distal tip of the elongated member. A visualization sensor, such as CMOS sensor, also has a polarizer directly in front of it, with this second polarizer being complimentary to the first polarizer so that any light reflected by the outer prism surface into the visualization sensor will be blocked by this polarizer. Light passing through the first polarizer and reflected by the surrounding tissue will have random polarization, so roughly half of this light will pass through the second polarizer to reach the visualization sensor and be converted to image data. The distal end polarized member may be a cover lens, e.g., for forward viewing elongated members, or a prism, e.g., for off-axis viewing elongated members, such as described in greater detail below and elsewhere in the specification.

In some instances, the distal end of the elongated member includes an off-axis visualization module that is configured so that the visualization sensor obtains data from a field of view that is not parallel to the longitudinal axis of the elongated member. With an off-axis visualization module, the field of view of the visualization sensor is at an angle relative to the longitudinal axis of the elongated member, where this angle may range in some instances from 5 to 90°, such as 45 to 75°, e.g., 30°. The off-axis visualization module may include any convenient light guide which collects light from an off-axis field of view and conveys the collected light to the visualization sensor. In some instances, the off-axis visualization module is a prism.

As summarized above, the internal tissue visualization devices of the invention further include a hand-held control unit to which the elongated member is operably connected. As the control unit is hand-held, it is configured to be held easily in the hand of an adult human. Accordingly, the hand-held control unit may have a configuration that is amenable to gripping by the human adult hand. The weight of the hand-held control unit may vary, but in some instances ranges from 0.5 to 5 lbs, such as 0.5 to 3 lbs. The hand-held control unit may have any convenient configuration, such as a hand-held wand with one or more control buttons, as a hand-held gun with a trigger, etc., where examples of suitable handle configurations are further provided below.

In some instances, the hand-held control unit may include a monitor. By monitor is meant a visual display unit, which includes a screen that displays visual data in the form of images and/or text to a user. The screen may vary, where a screen type of interest is an LCD screen. The monitor, when present, may be integrated or detachable from the remainder of the hand-held control unit. As such, in some instances the monitor may be an integrated structure with the hand-held control unit, such that it cannot be separated from the hand-held control unit without damaging the monitor in some manner. In yet other embodiments, the monitor may be a detachable monitor, where the monitor can be attached to and separated from the hand-held control unit, as desired, without damaging the function of the monitor. In such embodiments, the monitor and hand-held control unit may have a variety of different mating configurations, such as where the hand-held control unit includes a hole configured to receive a post of the monitor, where the monitor has a structure that is configured to snap onto a receiving structure of the hand-held control unit, etc. The monitor, when present will have dimensions sufficient for use with the hand-held control unit, where screen sizes of interest may include 10 inches or smaller, suches or smaller, e.g., 5 inches or smaller, e.g., 3.5 inches, etc.

Data communication between the monitor and the remainder of the hand-held control unit may be accomplished according to any convenient configuration. For example, the monitor and remaining components of the hand-held control unit may be connected by one or more wires. Alternatively, the two components may be configured to communication with each other via a wireless communication protocol. In these embodiments, the monitor will include a wireless communication module.

In some embodiments, the distal end of the elongated member is rotatable about its longitudinal axis when a significant portion of the hand-held control unit is maintained in a fixed position. As such, at least the distal end of the elongated member can turn by some degree while the hand-held control unit attached to the proximal end of the elongated member stays in a fixed position. The degree of rotation in a given device may vary, and may range from 0 to 3600, such as 0 to 270°, including 0 to 1800. Rotation, when present, may be provided by any convenient approach, e.g., through use of motors.

Of interest are devices in which the hand-held control unit is reusable. In such devices, the elongated member is configured to be detachable from the hand-held control unit. As the elongated member is configured to be readily separable from the hand-held control unit without in any way damaging the functionality of the hand-held control unit, such that the hand-held control unit may be attached to another elongated member. As such, the devices are configured so that the hand-held control unit can be sequentially operably attached to multiple different elongated members. Of interest are configurations in which the elongated member can be manually operably attached to a hand-held control unit without the use of any tools. A variety of different configurations may be employed, e.g., where the proximal end of the elongated member engages the hand-held control unit to provide an operable connection between the two, such as by a snap-fit configuration, an insertion and twist configuration, etc. In certain configurations, the hand-held control unit has a structure configured to receive the proximal end of the elongated member.

In some instances, the hand-held control unit may be re-used simply by wiping down the hand-held control unit following a given procedure and then attaching a new elongated member to the hand-held control unit. In other instances, to provide for desired sterility to the hand-held control unit, the device may include a removable sterile covering attached to the proximal end of the elongated member that is configured to seal the hand-held control unit from the environment. This sterile covering (e.g., in the form of a sheath as described in greater detail below) may be a disposable sterile handle cover that uses a flexible bag, a portion of which is affixed to and sealed to the proximal end of the disposable elongated member. Where desired, the sterile covering may include an integrated clear monitor cover, which may be rigid and configured to conform to the monitor screen. In some instances, the cover may be configured to provide for touch screen interaction with the monitor. As indicated above, the hand-held control unit may include a manual controller. In such instances, the sterile covering may include a flexible rubber boot for mechanical controller sealing, i.e., a. boot portion configured to associated with the manual controller. In addition, the sterile covering may include a seal at a region associated with the proximal end of the hand-held control unit. In these instances, the open side of sterile cover prior to use may be conveniently located at the proximal end. Following positioning of the cover around the hand-held control unit, the open side may be mechanically attached to the handle and closed by a validated sealing method. The sterile cover of these embodiments is configured such that when employed, it does not inhibit handle controls or elongated structure and monitor actuation.

In addition to the distal end integrated visualization sensor, e.g., as described in greater detail above, devices of the invention may include a distal end integrated non-visualization sensor. In other words, the devices may include one or more non-visualization sensors that are integrated at the distal end of the elongated member. The one or more non-visualization sensors are sensors that are configured to obtain nonvisual data from a target location. Non-visual data of interest includes, but is not limited to: temperature, pressure, pH, elasticity, impedance, conductivity, distance, size, etc. Non-visualization sensors of interest include those configured to obtain one or more types of the non-visual data of interest. Examples of sensors that may be integrated at the distal end include, but are not limited to: temperature sensors, pressure sensors, pH sensors, impedance sensors, conductivity sensors, elasticity sensors, etc. Specific types of sensors include, but are not limited to: thermistors, strain gauges, membrane containing sensors, MEMS sensors, electrodes, light sensors, etc. The choice of a specific type of sensor will depend on the nature of the non-visual data of interest. For example, a pressure sensor can detect the force applied to a target tissue as it is deformed to determine the elastic modulus of the target tissue. A temperature sensor can be employed to detect locally elevated temperatures (which can be used to differentiate different types of tissue, such as to different normal and tumor tissue (where tumors exhibit increased bloodflow and therefore a higher temperature)). A properly collimated laser beam could be used to determine the distance to objects in the device field of view or the length scale of objects in the device field of view. When present, the integrated non-visualization sensor or sensors may be configured to complement other distal end components of the devices, so as to minimize any impact on the outer dimension of the distal end, e.g., in ways analogous to those described above in connection with integrated illumination elements.

In some embodiments, the tissue modifier is not a tissue modifier that achieves tissue modification by clamping, clasping or grasping of tissue such as may be accomplished by devices that trap tissue between opposing surfaces (e.g., jaw-like devices). In these embodiments, the tissue modification device is not an element that is configured to apply mechanical force to tear tissue, e.g., by trapping tissue between opposing surfaces.

In some instances, the tissue modifier is a low-profile tissue modifier, such as a low-profile biopsy tool or a low-profile cutter. Such low-profile tissue modifiers are include tissue cutting structure positioned at the distal of the elongated member. Because the biopsy or cutting tool is low-profile, its presence at the distal end of the elongated member does not substantially increase the outer diameter of the elongated member. In some instances, the presence of the low-profile biopsy tool increase the outer diameter of the elongated member by 2 mm or less, such as 1.5 mm or less, including 1 mm or less. The configuration of the low-profile biopsy tool may vary. In some instances, the low-profile biopsy tool comprises an annular cutting member concentrically disposed about the distal end of the elongated member and configured to be moved relative to the distal end of the elongated member in a manner sufficient to engage tissue. The annular cutting member may or may not be configured as a complete ring structure, where the ring structure is movable in a longitudinal manner relative to the distal end of the elongated member (such that it may be, moved along the elongated member towards and away from the proximal end of the elongated member). The distal edge of the ring structure may be movable some distance beyond the distal end of elongated member, where this distance may vary and in some instances is 10 mm or less, such as 5 mm or less, including 3 mm or less. The distal edge of the ring structure may be sharp in order to penetrate tissue, and may include one or more tissue retaining structures, such as barbs, hooks, lips, etc., which are configured to engage the tissue and stably associate the engaged tissue with the ring structure, e.g., when the ring structure is moved longitudinally along the elongated member towards the proximal end. Also of interest are cutting tools, e.g., as described.

In some instances, these may include a collimated laser configured to emit collimated laser light from a distal region of the elongated member, such as the distal end of the elongated member. The collimated laser components of these embodiments may be configured for use for a variety of purposes, such as but not limited to: anatomical feature identification, anatomical feature assessment of sizes and distances within the field of view of the visualization sensor, etc.

The devices of the invention may be fabricated using any convenient materials or combination thereof, including but not limited to: metallic materials such as tungsten, stainless steel alloys, platinum or its alloys, titanium or its alloys, molybdenum or its alloys, and nickel or its alloys, etc.; polymeric materials, such as polytetrafluoroethylene, polyimide, PEEK, and the like; ceramics, such as alumina (e.g., STEATITE™ alumina, MAECOR™ alumina), etc.

In some instances, the devices may include a stereoscopic image module. By stereoscopic image module is meant a functional module that provides a stereoscopic image from image data obtained by the device. As such, the module provides a user via the monitor with the perception of a three-dimensional view of an image produced from the image data obtained by the device. The module is described in terms of “images”, and it should be understood that the description applies equally to still images and video. Further details regarding stereoscopic image modules and image recognition modules can be found in U.S. application Ser. Nos. 12/501,336 and 12/269,770; the disclosures of which are herein incorporated by reference.

Where the device includes a stereoscopic image module, the device may include two or more distinct visualization sensors (e.g., CMOS cameras as reviewed above) or a single visualization sensor via which the image data is collected and employed by the stereoscopic image module to provide the stereoscopic image. Where the elongated member includes first and second visualization sensors, the stereoscopic imaging module is configured to process imaged data provided by the first and second visualization sensors to produce the stereoscopic image. In such embodiments, any convenient stereoscopic image processing program may be employed. FIG. 17 illustrates a block flow diagram of a technique to produce stereoscopic images from image data, according to one embodiment. Left and right image data are obtained (as represented by blocks 1005), either sequentially from a single visualization sensor that is moved from a first position to a second position or, if two visualization sensors are present, sequentially or simultaneously. The left and right Image data account for the different locations and perspectives associated with each respective position of the same visualization sensor or respective positions of the two distinct visualization sensors. The image data for the first and second images may include distortions, and an algorithm may be employed, for example, in which the left and right image data are first warped as shown via a calibration element to remove lens distortion, as represented by blocks 1010. Any convenient algorithm may be employed. Algorithms of interest include those described in “Geometric Calibration of Digital Cameras through Multi-view Rectification” by Luca Lucchese (Image and Vision Computing, Vol. 23, Issue 5, May 2005, pp. 517-539); and Levenberg-Marquardt algorithm, “Correction of Geometric Lens Distortion through Image Warping” by Lucchese (ISPA 2003, Proceeding of the 3rd International Symposium on Image and Signal Processing and Analysis, 18-20 Sep. 2003, Vol. 1, pp. 516-521). The resultant undistorted left and right images, represented by blocks 1015, are then processed with stereo and image fusion algorithms to construct a stereoscopic image, as represented at blocks 1020, 1022, 1024, 1026, 1028. Any convenient stereo and image fusion algorithms may be employed, such as but not limited to those described in: “Scene Reconstruction from Multiple Cameras” by Richard Szeliski (Microsoft Vision Technology Group; see also, http://research.microsoft.com/pubs/75687/Szeliski-ICIPOO.pdf); “A parallel matching algorithm for stereo vision”, by Y. Nishimoto and Y. Shirai (IJCAI-1985-Volume 2, pg. 977; see also, http://ijcai.org/Past%20Proceedings/IJCAI-85-VOL2/PDF/059.pdf); “Image Fusion Using Wavelet Transform”, by Zhu Shu-long (Institute of Surveying & Mapping; Commission IV, Working Group IV/7; see also, http://www.isprs.org/commission4/proceedings02/pdfpapers/162.pdf); “Disparity field and depth map coding for multiview 30 image generation”, by D. Tzovaras (Image Communication, Signal Processing; 1998, vol. 11, n*3, pp. 205-230); etc.

Stereo algorithms compute range information to objects seen by the visualization sensors by using triangulation. Objects seen at different viewpoints will result in the object at different locations in the image data for the first and second visualization sensors. The disparity, or image difference, is used in determining depth and range of objects. Corresponding pixel points within the image data for the first and second visualization sensors may be identified and used in the determination of disparity line, as represented by block 1 024. Because the first and second visualization sensors are at different locations and hence have different perspectives, the same object present in image data for the first and second visualization sensor may be at different pixel coordinate locations. Triangulation may be implemented, as represented by block 1026, based on geometry associated with the locations of the first and second visualization sensors may be used to determine depth and range of objects seen by the visualization sensors. Triangulation computations are applied to derive range data, and the resultant range (or depth) map can be overlayed on the image display, as desired. This is represented at block 1028 in FIG. 17. Stereoscopic images taking into account three-dimensional depth information can thus be reconstructed from image data from the first and second visualization sensor.

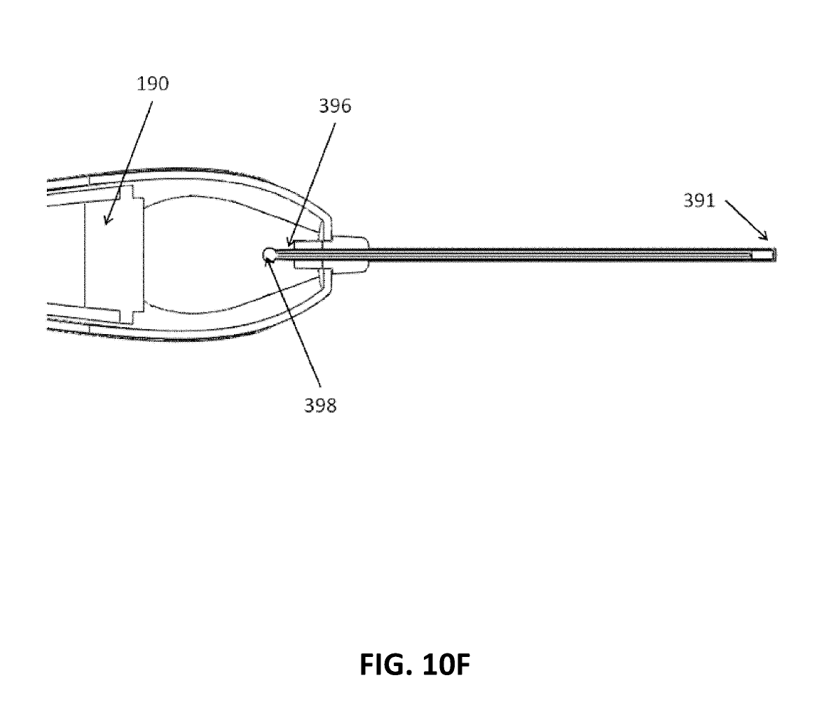

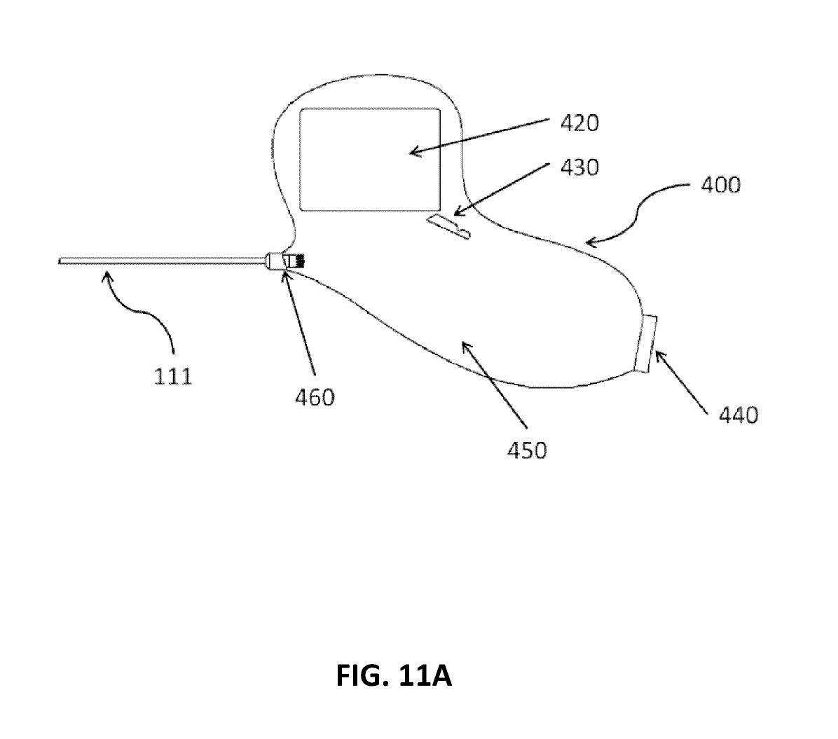

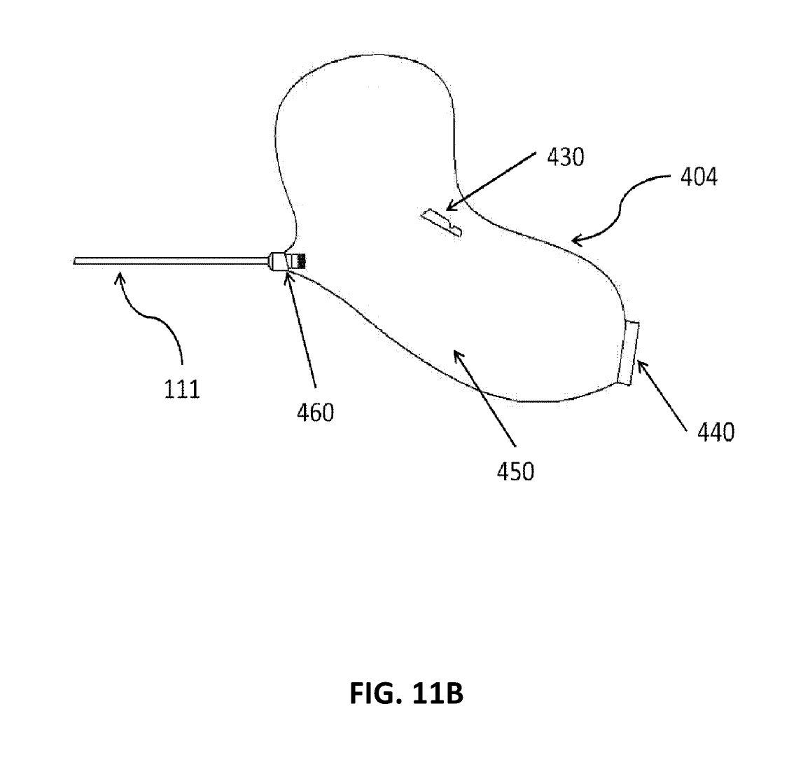

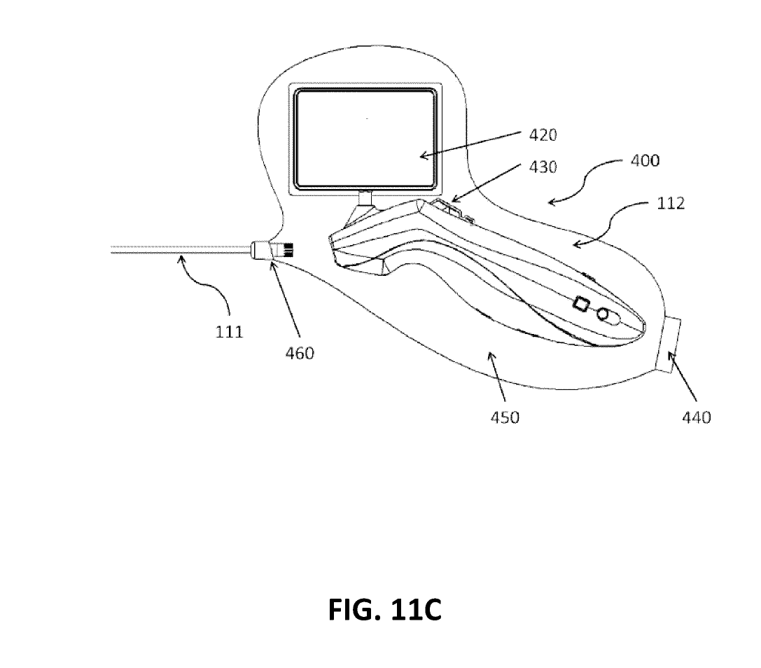

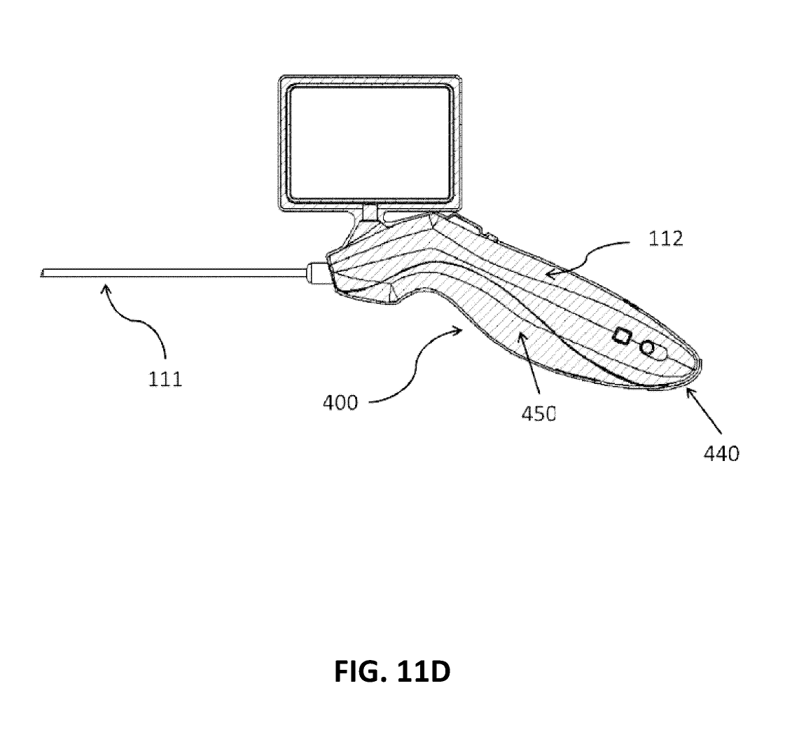

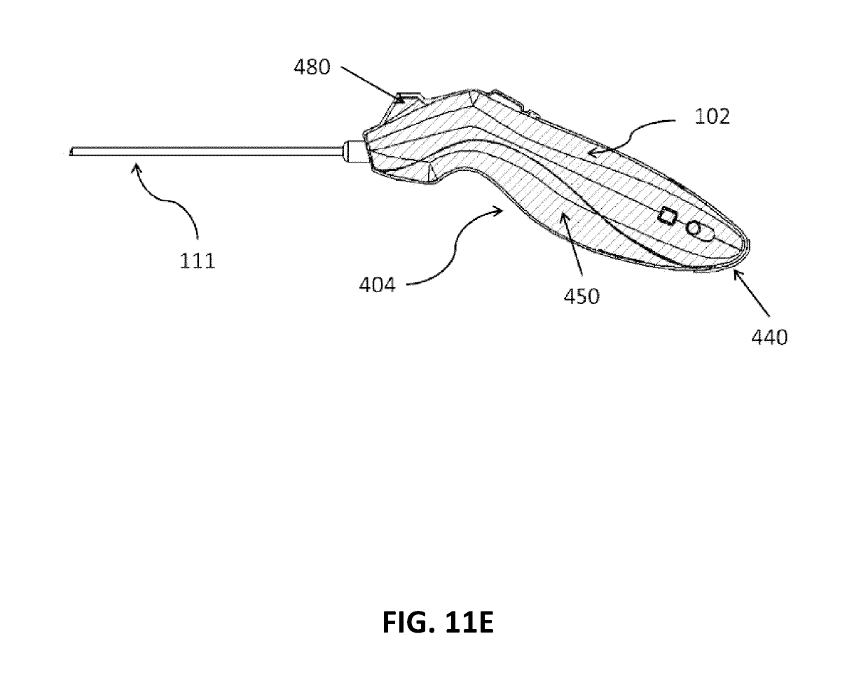

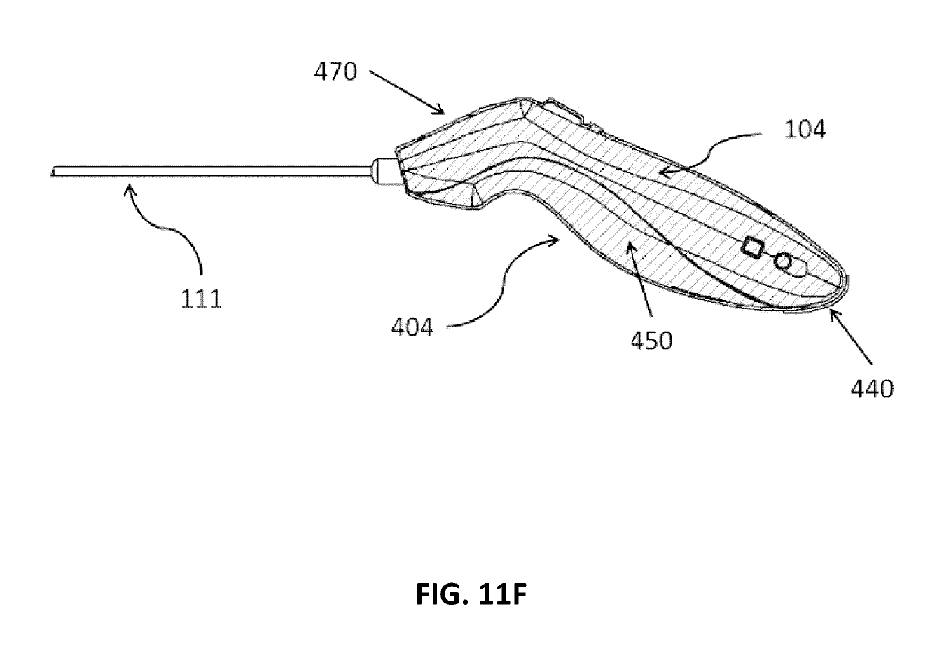

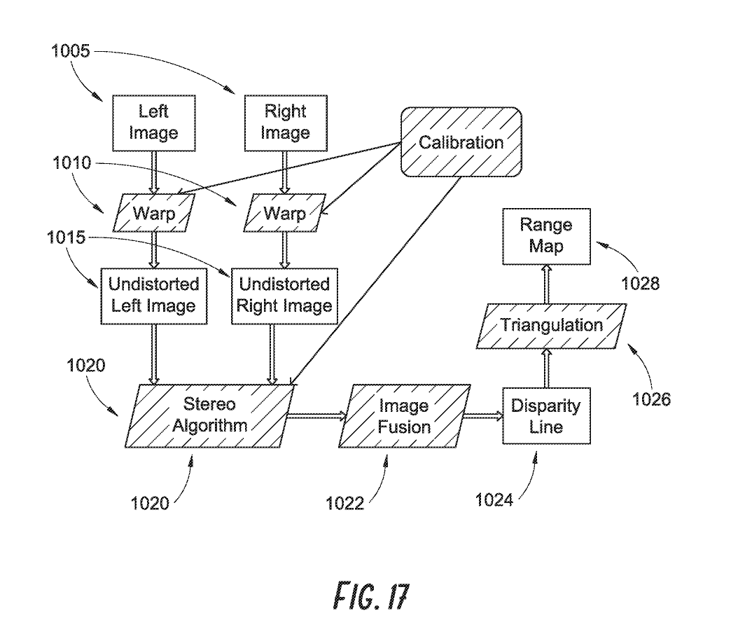

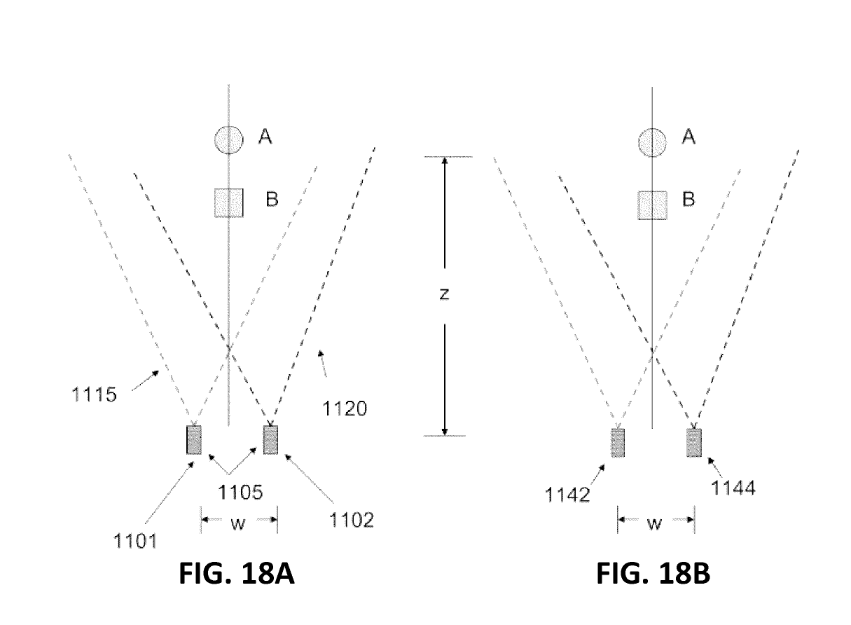

FIG. 18B illustrates slightly offset visualization positions, according to certain embodiments. FIG. 18B illustrates two visualization sensors, i.e., 1142 for a first view of objects A and B and 1144 for a second view of objects A and B. The depth and range of the object is found in a similar manner as for FIG. 11A, as described in more above.

Further details regarding aspects of stereoscopic image modules that employ image data obtained by two or more distinct visualization sensors may be found in U.S. application Ser. No. 12/269,770; the disclosure of which is herein incorporated by reference.

Also of interest are stereoscopic image modules that are configured to provide a stereoscopic image from data obtained by a single image sensor. In such embodiments, the image sensor is configured to provide to the stereoscopic image module consecutive offset image data of the target tissue location, which consecutive offset image data are then employed by the stereoscopic image module to provide the desired stereoscopic image. By consecutive offset image data is meant image data that includes at least data from a first view of a target tissue location and data from a second view of the same target location, where the second view is offset from the first view. The second view may be offset from the first view by any convenient distance, for example 1 mm or less, including 0.5 mm or less; The first and second offset views may be obtained using any convenient approach. In one approach, the single visualization sensor is moved from a first position to a second position in order to obtain the desired offset image data. The single visualization sensor may be moved from the first to the second positions using any convenient manner, e.g., by a mechanical element that physically moves the sensor from the first to the second position. In yet other embodiments, the desired offset views may be obtained with a single visualization sensor operatively coupled to an optical guide system (which may include one or more of lenses, mirrors, filters, etc.) configured to provide the desired first and second offset views. For example, the first and second offset views may be provided to the single visualization sensor by including a first and second lens systems which alternately convey image data to the visualization sensor. The offset views may also be provided, for example, by including a single lens system with mirrors configured to provide the lens with two or more different views. The frequency with which the first and second offset views are obtained may vary, where in some instances the frequency may range from 1 to 30 frames/sec, such as 1 to 15 frames/sec. Various systems may be implemented to provide multiple views with a single camera. Systems of interest include, but are not limited to, those described in: “Scalable Multi-view Stereo Camera Array for Real World Real-Time Image Capture and Three Dimensional Displays” by S. Hill (Massachusetts Institute of Technology, Program in Media Arts and Sciences School of Architecture and Planning; May 7, 2004; see also, http://web.media.mit.edu/-vmb/papers/hillms.pdf); “Single Camera Stereo Using Planar Parallel Plate” by Chunyu Gao, et al. (Beckman Institute, University of Illinois at Urbana-Champaign; see also, http://vision.ai.uiuc.edu/newpubs/Stereo PPP Gao.pdf); and, “3-D Reconstruction Using Mirror Images Based on a Plane Symmetry Recovering Method” by Mitsumoto, H., et al. (IEEE Transaction on Pattern Analysis and Machine Intelligence; Vol. 14; Issue No. 9, September 1992, pp. 941-946).

FIG. 18A illustrates a single visualization sensor 1105 which is moved to two different positions (11 01 and 11 02) to sequentially obtained image data, which sequentially obtained image data is employed by a stereoscopic image module to produce a stereoscopic image of objects A and B. The first and second visualization positions 1101 and 1102 are at an offset width W from one another, which may vary, ranging in some instances from 1 mm or less, such as 0.5 mm or less. Objects A and B located at a focal plane distance Z are seen at different perspectives for the first and second positions (shown by dotted lines 1115, 1120, respectively). The difference in viewing perspectives is reflected in the image data obtained by the single image sensor from the first and second positions. As shown, first visualization sensor 1105 sees objects A & B off to the right of center when in position 1101 and sees objects A and B off to left of center when in position 1102. The disparity between the two views is used to determine depth and range of objects A and B.

The stereoscopic image module may be implemented in a video processor module configured to receive image data obtained by the one or more visualization sensors. The stereoscopic image module processes the image data to provide stereoscopic image data for display on a display.

In certain embodiments, devices of the invention include an image recognition module. Image recognition modules of interest are those that are configured to receive image data and compare the received image data with a reference that includes at least one of color descriptor data and anatomical descriptor data to make a determination as to whether an alert signal should be generated. The term “reference” is used herein to refer to data in any format, e.g., saved as one or more image files, etc., that is for one or more reference images, e.g., where the data can be used by an appropriate processor to produce one or more reference images. As such, a reference includes at least a first set of reference image data for a first reference image. In some instances a reference also includes a second set of reference image data for a second reference image. In such embodiments, a reference may include sets of reference image data for multiple reference images, e.g., 2 or more, 5 or more, 10 or more, 25 or more, 50 or more, 100 or more, 1000 or more, 1500 or more, 2000 or more, 5000 or more, 10,000 or more etc., reference images. Further details regarding image recognition modules are provided in U.S. application Ser. Nos. 12/501,336 and 12/437,186; the disclosures of which are incorporated by reference.

Reference images are predetermined images of a region of interest. As the reference images are predetermined, they are images that have been produced independently of the image data that is received by the image processing module. In some instances, the reference images are images that exist prior to obtainment of the image data that is received by the image processing module. The reference images may be images that are obtained from the same subject (e.g., person) that is being visualized during a given procedure (e.g., where the reference images were obtained from the subject prior to a given procedure) or from a different subject (e.g., person). Alternatively, the reference images may be produced de novo, such that they are not produced from image data obtained from any actual subject but instead are designed, e.g., by using manual or computer assisted graphic protocols.

Reference images that make up the reference may differ from each other in a number of ways. For example, any two given reference images may be images of regions of interest of different internal tissue locations. In such a reference, the reference may include first and second pre-determined images that differ from each other with respect to a pre-determined internal tissue location. For example, the reference may include images of at least a first tissue location and a second tissue location. The first and second tissue locations may be locations that a given device may be expected to image during a given procedure, such as during a surgical procedure. In some instances, the reference includes multiple images of different locations that a given visualization sensor should image during a given procedure if the procedure is performed correctly. The reference may also include images of different tissue locations that a visualization sensor should not see during a given procedure, e.g., images of tissue locations that should not be viewed by the sensor if the given procedure of interest is being performed correctly. Accordingly, some references may include multiple images that track the location of a device when correctly and incorrectly positioned during an entire procedure, such as an entire surgical procedure.

The sets of image data in the reference may include one or more color descriptor data and anatomical descriptor data. By color descriptor data is meant data which is based on the particular color of a given internal tissue site and components thereof. For example, an internal tissue site may include one or more tissues that each has a distinct color. For example, different tissues such as muscle, nerve, bone, etc., may have different colors. This distinct color may be present in the reference image as' color descriptor data, and employed by the image processing module. By anatomical descriptor data is meant data which is based on the particular shape of one or more tissue structures at the internal tissue site. For example, different tissues such as muscle, nerve, bone, etc., have different shapes. These different shapes are present in the image data as anatomical descriptor data.

As summarized above, the image recognition module compares received image data of an internal tissue site (e.g., obtained during a given procedure of interest) with the reference. The comparison performed by the image recognition module may be achieved using any convenient data processing protocol. Data processing protocols that may be employed in this comparison step may compare the received image data and reference based on color descriptor data and/or anatomical descriptor data. Data comparison protocols of interest include, but are not limited to: mean absolute difference between the descriptors of data and stored values such as mean color intensity, and, the degree of correlation between principle axis of the structure and stored values.

In performing this comparison step, the image recognition module may be configured to automatically select the appropriate images from a reference to compare against the received image data. In some instances, the image recognition module is configured to compare the received image data with the reference by selecting an appropriate set of reference image data based on a determined positional location of the device. For example, the image recognition module may obtain positional information about the device (e.g., as may be obtained from sensors on the device or manually input and associated with a given image) and then select reference images that are for the same positional location as the device when the device obtained the image data being received. Alternatively, the image recognition module may automatically select appropriate sets of image data based on similarity parameters. For example, the image recognition module may automatically select the most similar sets of image data from the reference to use in the comparison step.

The image recognition module compares the received image data with the reference in order to determine whether an alert signal should be generated. In other words, the output of the image recognition module is a decision as to whether an alert signal should be generated. If an image recognition module determines that an alert signal should be generated, it may generate the alert signal or instruct a separate module of the system to produce an alert signal.

The alert signal, when generated, may vary depending on the nature of the system. An alert signal may be a warning signal about a given system parameter or a signal that confirms to an operator of the system that a given system parameter of interest is acceptable. In some embodiments, an alert signal may include functional information about a device. For example, in these embodiments an alert signal may include information that a given device is functioning properly. In some embodiments, an alert signal may include positional information about a device. For example, an alert signal may include information as to whether or not a given device is correctly spatially positioned. In these embodiments, the alert signal may contain information that a tissue modifier of the device is contacting non-target tissue, such that the tissue modifier is not correctly spatially positioned.

The system may be configured to employ an alert signal in a variety of different ways. The system may be configured to provide the alert signal to a user of the system, e.g., via an alert signal output of the system. In addition or alternatively, the system may be configured to automatically modulate one or more operational parameters of the system based on the generation of an alert signal. For example, where the image processing module determines that a tissue modifier is contacting non-target tissue and therefore generates an alert signal, the alert signal may automatically modulate operation of the tissue modifier, e.g., by turning it off. In some instances, the alert signal may automatically shut the system down.

Further details regarding image recognition modules are provided in U.S. application Ser. No. 12/437,186; the disclosure of which is herein incorporated by reference.

The stereoscopic module and image recognition modules, e.g., as described above, may be implemented as software, e.g., digital signal processing software; hardware, e.g., a circuit; or combinations thereof, as desired.

In some embodiments, the devices may include a conveyance structure configured to convey an item between the distal end of the elongated member and an entry port positioned at a proximal end of the device, e.g., associated with the proximal end of the elongated member or associated with the hand-held control unit. This conveyance structure may have any convenient configuration, where in some instances it is a “working channel” disposed within the elongated member. When present as a working channel, the channel may have an outer diameter that varies, and in some instances has an outer diameter of 3 mm or less, such as 2 mm or less and including 1 mm or less. The conveyance structure may be configured to transport items, e.g., fluids, medicines, devices, to an internal target site or from an internal target site. As such, the proximal end entry port of the conveyance structure may vary, and may be configured to be operably coupled to a variety of different types of components, such as but not limited to: aspiration units, fluid reservoirs, device actuators, etc. As indicated elsewhere, devices of the invention may be configured for wireless data transmission, e.g., to provide for one or more of: transmission of data between various component of the device, transmission of data between components of the device and another device, such as hospital information system, separate monitor, etc. Any convenient wireless communication protocol may be employed, where in some instances wireless communication is implemented as one or more wireless communication modules.

A video processor module may be present and be configured to control the one or more distinct visualization sensors by sending camera control data to a camera module including the visualization sensor(s). The video processor may also be configured to receive sensor data from one or more sensors and/or tools; and further, may be configured to control the sensors and/or tools by sending sensor control data to a sensor module including the one or more sensors and/or tools. The various sensors may include, but are not limited to, sensors relating to pressure, temperature, elasticity, ultrasound acoustic impedance, laser pointer to identify and/or measure difference to sensors, etc. The various tools may include, but are not limited to, a measurement scale, teardrop probe, biopsy probe, forceps, scissors, implant device, IR lighting, ultrasound measurement device, cutting tool, etc. Depending on the specific application and sensor/tool implemented, sensor data may also be included with the image data for processing by the stereoscopic image module, in order to provide the stereographic images.

In certain instances, the devices of the invention include an updatable control module, by which is meant that the devices are configured so that one or more control algorithms of the device may be updated. Updating may be achieved using any convenient protocol, such as transmitting updated algorithm data to the control module using a wire connection (e.g., via a USB port on the device) or a wireless communication protocol. The content of the update may vary. In some instances, a hand-held control unit is updated to configure the unit to be used with a particular elongated member. In this fashion, the same hand-held control units may be employed with two or more different elongated members that may differ by function and have different components. In some instances, the update information may be transmitted from the particular elongated member itself, such that upon operable connection of the elongated member to the hand-held control unit, update information is transferred from the elongated member to the hand-held control unit that updates the control module of the hand-held control unit such that it can operate with that particular elongated member. The update information may also include general functional updates, such that the hand-held control unit can be updated at any desired time to include one or more additional software features and/or modify one or more existing programs of the device. The update information can be provided from any source, e.g., a particular elongated member, the internet, etc.

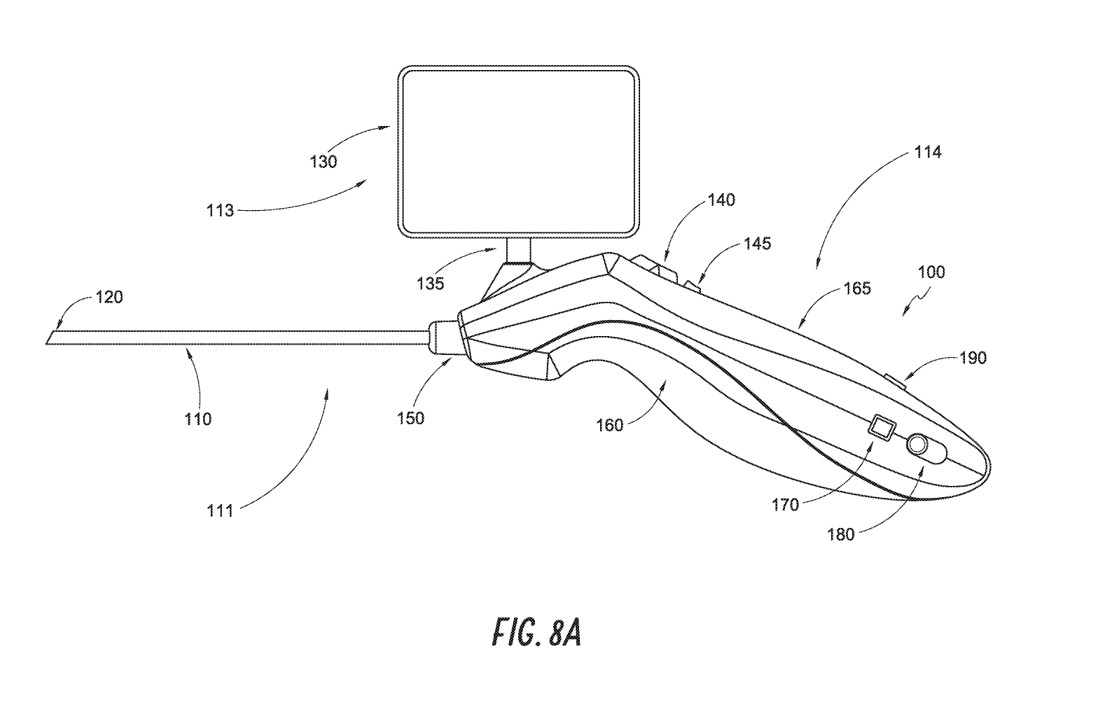

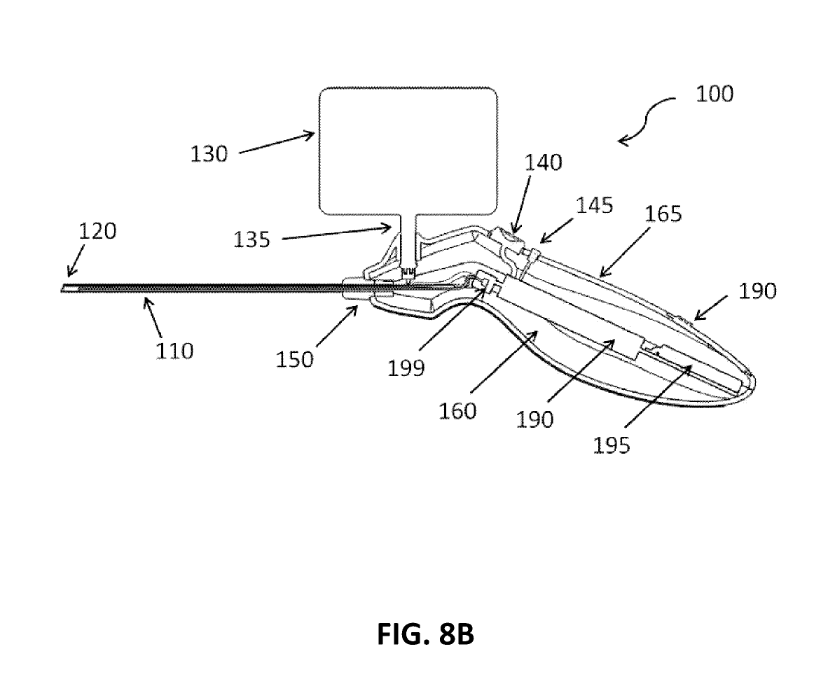

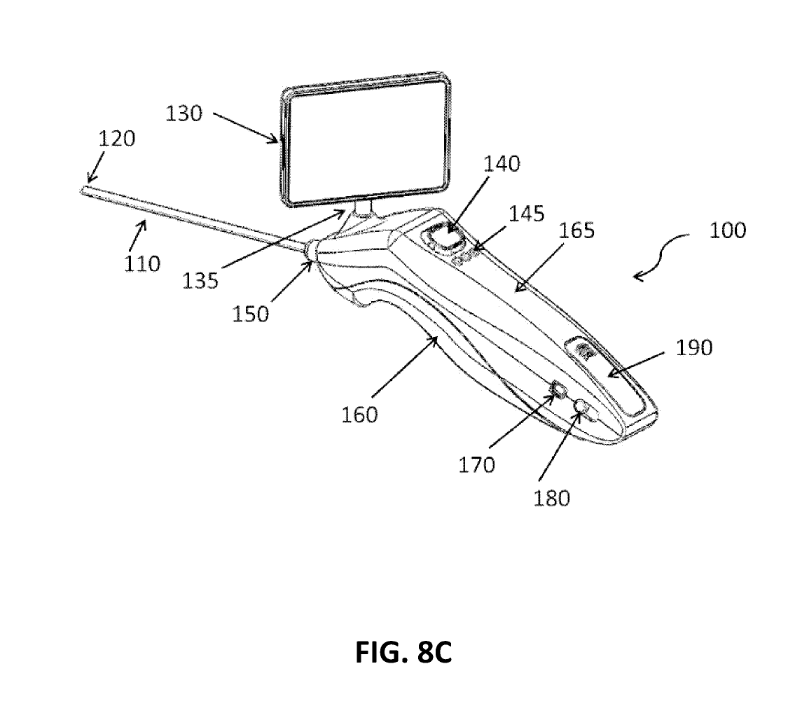

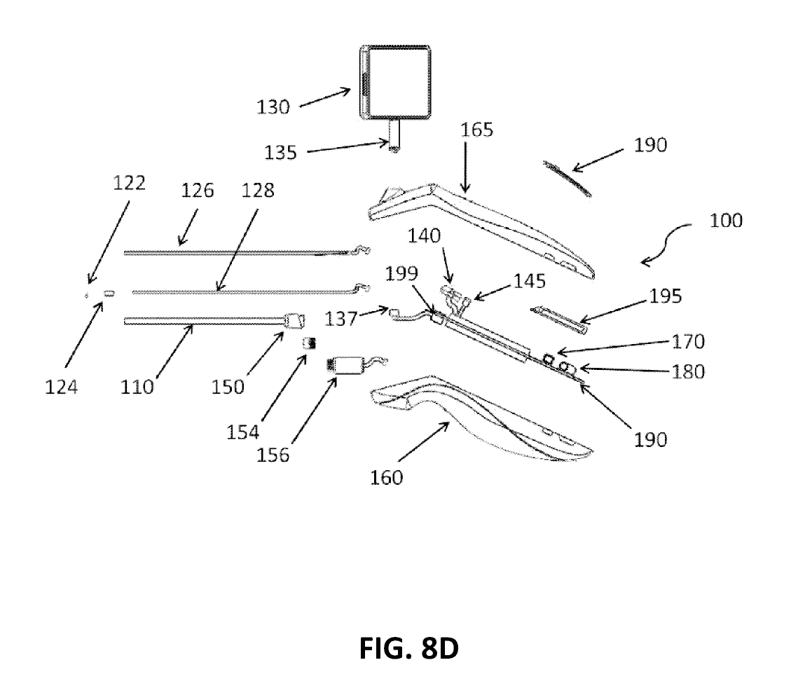

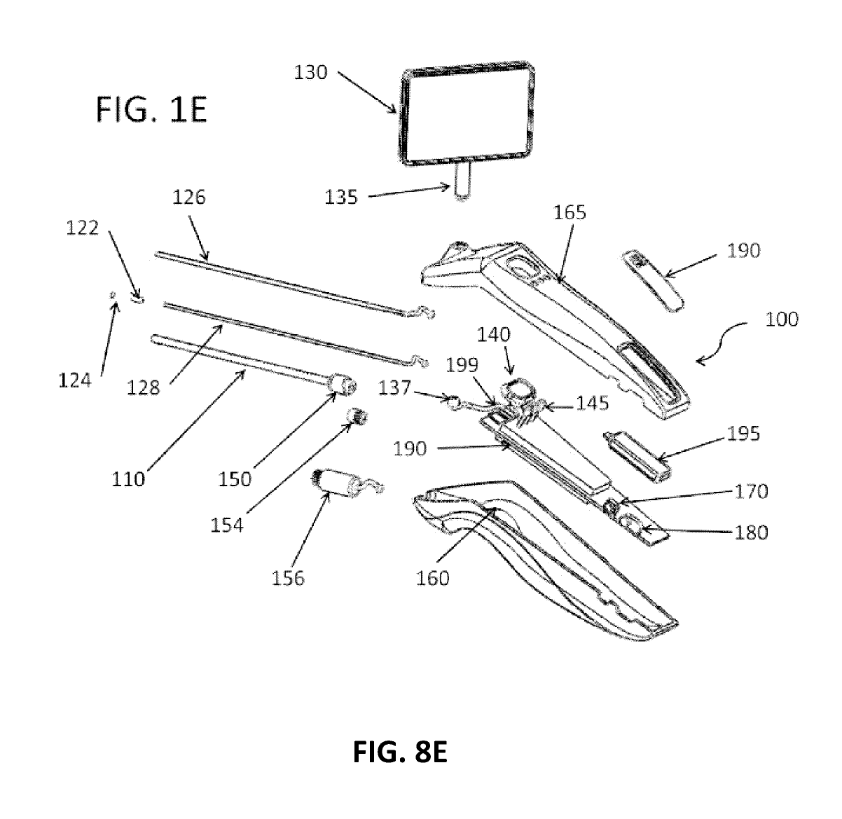

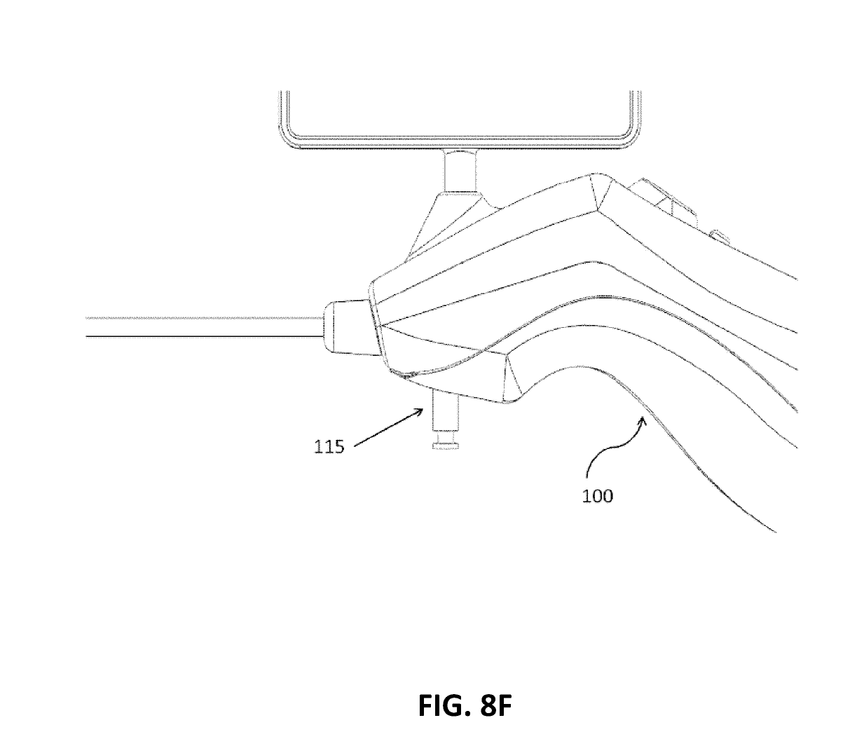

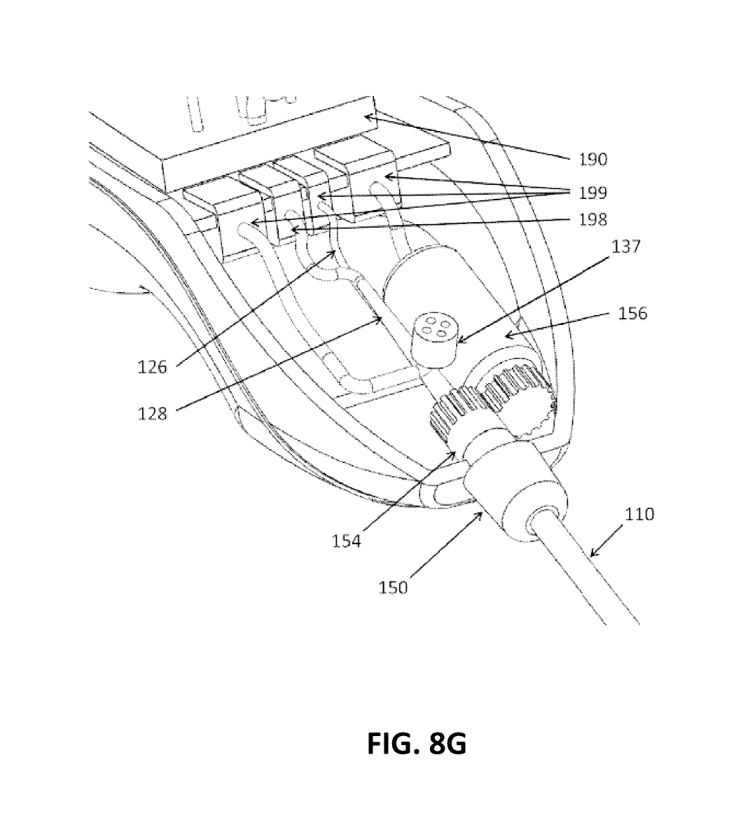

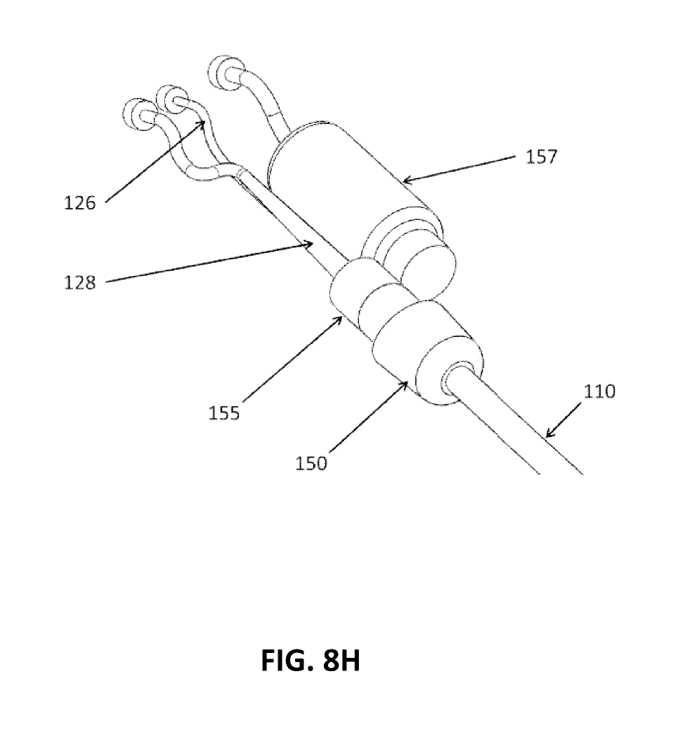

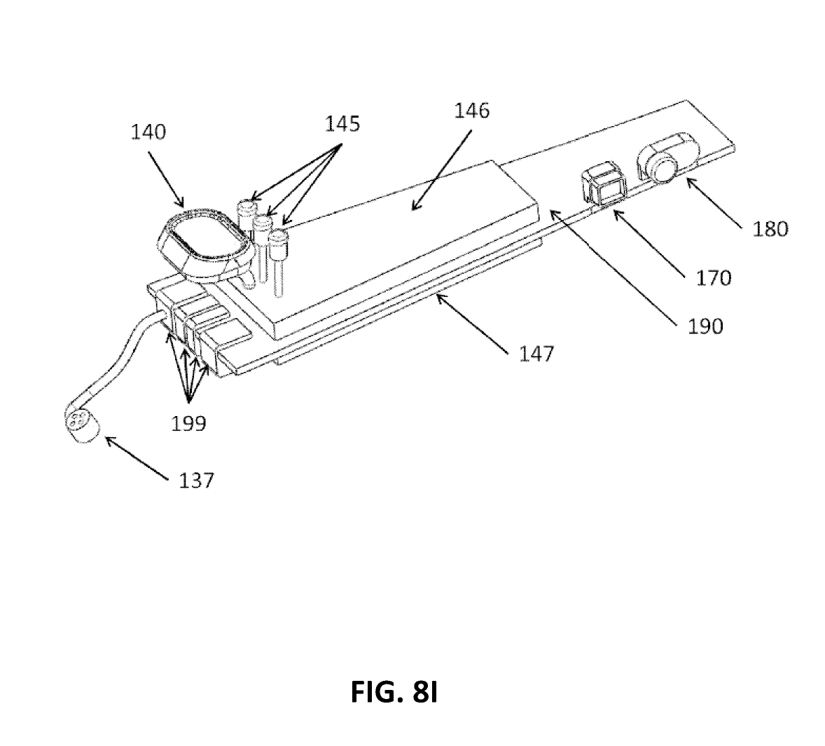

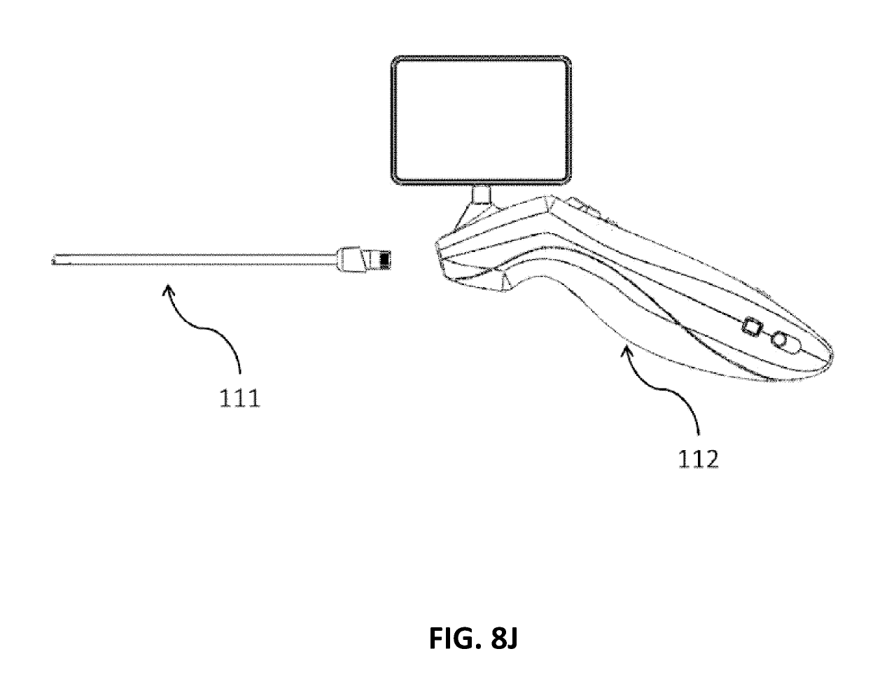

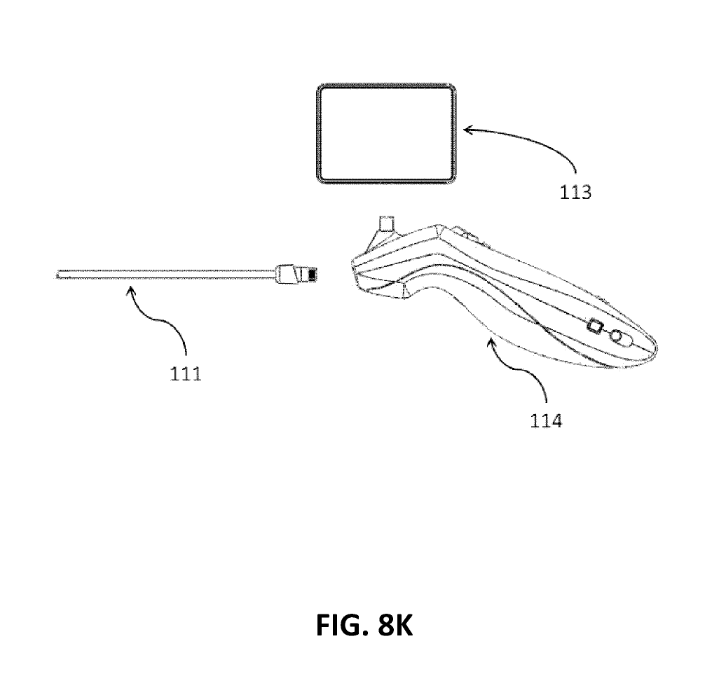

Turning now to the figures, FIGS. 8A-8K, illustrate one embodiment a self-contained, portable diagnostic imaging device of the invention. The hand-held, self-contained, portable diagnostic imaging device 100 illustrated in these figures includes a hand piece 114 and a removably attached elongated member 111 having a distal end integrated CMOS sensor, which is referred to herein as a “probe piece.” See FIG. 8K.

From an external view, the probe piece, as shown in FIGS. 8A and 8C, includes a distal tip 120, an elongated tubular structure 110, and a mechanical connector 150 to the hand piece. The hand piece, from an external view, as shown in FIGS. 8A and 8C, includes a rotatable and removable monitor unit 113 made up of a monitor 130 and a monitor mount 135 that may be attached to either the monitor housing or the top part of the hand piece depending on the embodiment, a single port 170, such as a USB port, for use as an input for programming or as an output for video and still images, an on/off switch 180 for managing power input to the device, a top cover 165, a bottom cover 160, switches for image capture and data transfer and control 145, and a switch for controlling the rotation of the probe piece 140. This switch 140 generally has three positions for controlling the motor rotation, one position to rotate the motor clockwise, one position to rotate the motor counterclockwise, and a position in the center that is neutral. Lastly, as shown in FIGS. 8D and 8E, there is a battery door 190 for the purpose of accessing the battery 195.

Internally viewed, the device additionally contains a battery 195 that may be rechargeable, an electronic control board 190, and connectors 199 for all electrical and optical components of the device, to and from the electronic control board 190, as shown in FIG. 8B.

Within the distal tip 120 of the probe piece, as shown in FIGS. 8D and 8E, is a lens 122, such as a prism lens, or a flat lens (e.g., cover glass), and a CMOS visualization sensor (referred to herein as a camera) 124. Within the elongated structure portion 110 of the probe piece is a wire 128 for electrically connecting the camera 124 to a connector 199 on the electronics board 190. Also, an illuminator 126 is arranged within the probe piece so as to provide lighting at the distal tip 120, and is connected to the electronic control board 190 at the connectors 199.

Also within the hand piece, in the present embodiment of the invention as shown in FIGS. 8D, 8E and 8G, is a geared motor 156. Geared motor 156 is connected to the probe piece via a geared intermediary piece 154. The connection between the geared motor 156 and the intermediary piece 154 of the probe piece is oriented in such a way as to allow for the rotation of the probe piece both counterclockwise and clockwise. The connector 150 linking the probe piece to the hand piece does not rotate with the intermediary piece 154.

In another embodiment, as shown in FIG. 8H, there may be a frictional and rotational connection accomplished between the probe piece and the motor 157 by an intermediary piece 155, for example, a rubber to rubber contact connection. Both the motor 157 and the intermediary piece 155 are oriented in such a way as to allow for the rotation of the probe piece both counterclockwise and clockwise. The connector 150 linking the probe piece to the hand piece does not rotate with the intermediary piece 155.

Lastly, referring to FIGS. 8E and 8F, within the hand piece, there is a connector 137 for electrically coupling the monitor mount 135 to the electronic board 190. The connector 137 is configured to allow for the rotation of the monitor mount 135, and thus the monitor 130 connected to the monitor mount 135, without binding, breaking or kinking of the connector 137 or the associated wiring that connects the connector 170 to the electronic board 190.

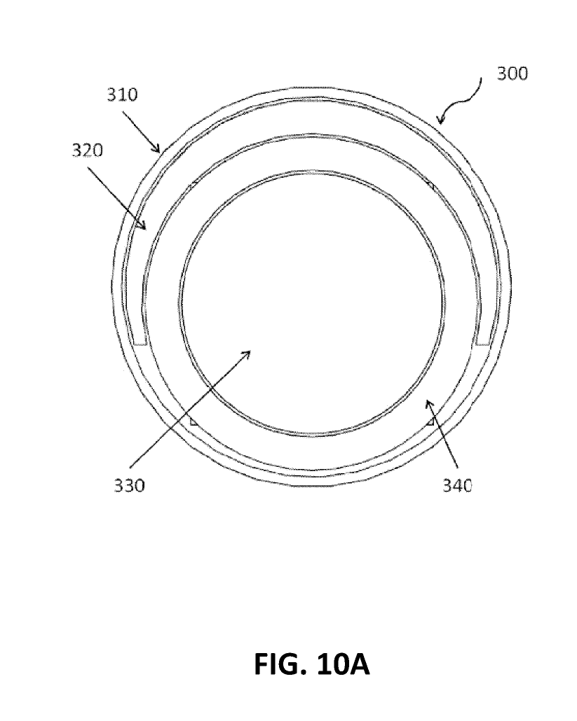

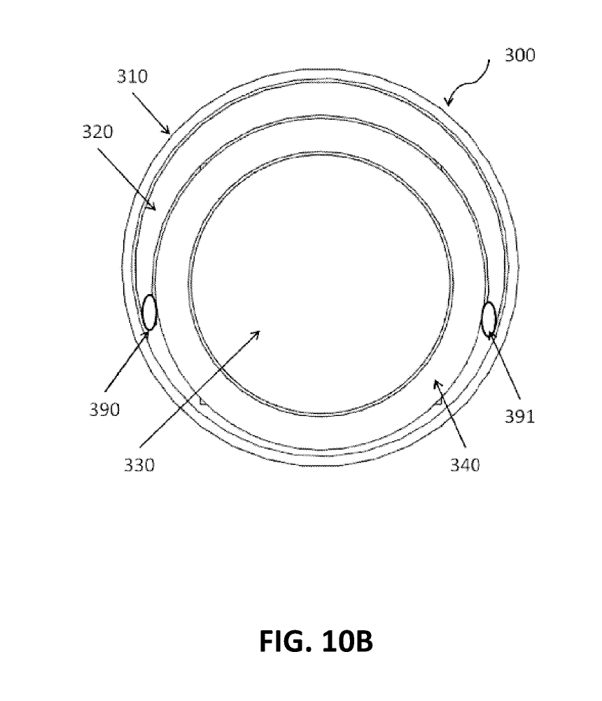

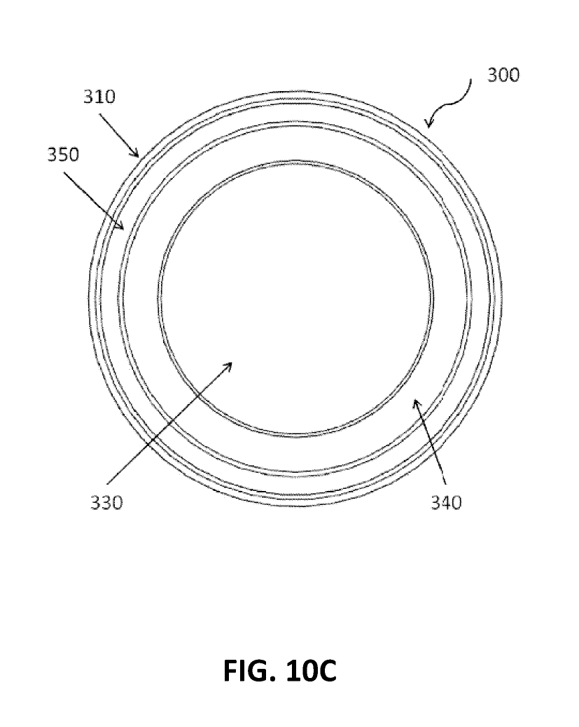

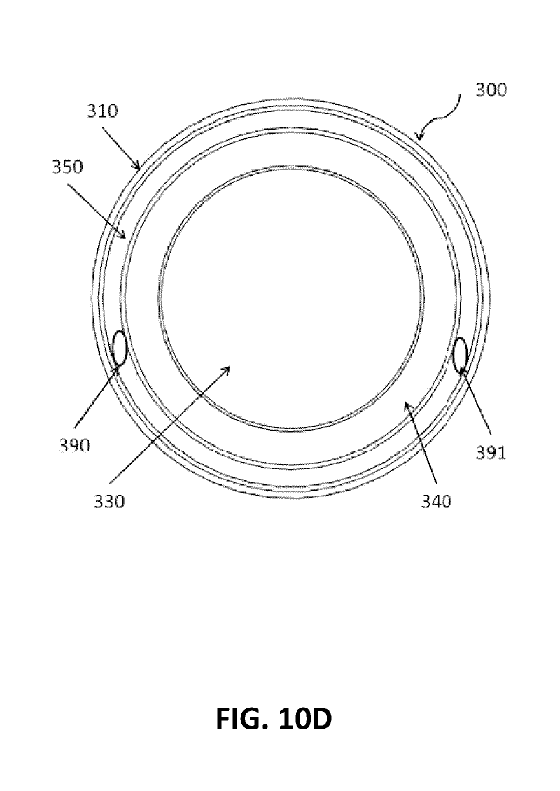

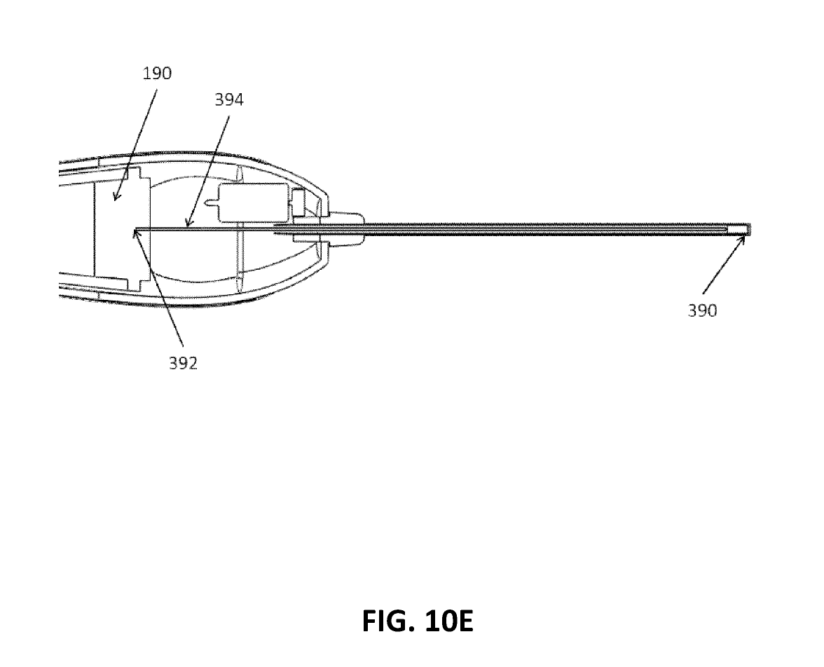

In another embodiment of the invention, the portable diagnostic imaging system 100 may include an element to transport material, medicine and implants to and from a point external to the hand piece and external to the distal tip 120 of the probe piece, e.g., a lumen configured as a working channel. As shown in FIG. 8F, there is a port connection 115, such as a luer connector for connecting to other luer connectors, for example a barbed connector for connecting to tubing, like a compression connector for connection to tubing. This port connector 115 may be located and protrude from either external half of the hand piece 165 and 160, and at any location convenient to the use of the device. Internal to both the hand piece and the probe piece is a conduit that connects the port 115 to a port 391, as shown in FIGS. 10B and 10D located at the very distal end of the distal tip 120 of the probe piece whereby a material, medicine or implant may be delivered from the hand piece 100. In another embodiment, the material, medicine or implant, may be aspirated into the port 391 at the distal tip 120 of the probe piece, and be transported through a conduit within the probe piece and hand piece, exiting through the port 115 located on the hand piece.

As mentioned above, devices of the invention may include an electronic board 190. FIG. 8I shows one embodiment of an electronic board 190 and its associated components. Generally speaking, one group of components that the electronic board 190 has electrically attached to it are electronic components of the control circuitry represented as blocks 146 and 147. In the example of FIG. 8I, there are two locations for electronic components 146 and 147 on the electronic board 190, but there may only be required, in other embodiments of the invention, electronic components located on one side or the other of the electronic board 190, and not necessarily to the footprint of the electronic components 146,147 as suggested in FIG. 8I.

Another item that is electrically attached to the electronic board 190 is an electrical connector 170 for transmitting data to and from the electronic board 190 to an external transmitting or receiving means. In one embodiment of the present invention, the electrical connector 170 may be used to program a chip that may be located in the electronic component area or areas of 146 and/or 147 of the electronic board 190, for example with a computer. In another embodiment, the electrical connector 170 may be used for downloading video or still images that are captured by the camera that is located at the distal tip 120 of the probe piece means and stored in a memory chip that may be located in the electronic component area or areas of 146 and/or 147 of the electronic board 190. Additionally this memory chip may be removable from the present invention or reattached to the present invention. In another embodiment of the present invention the electronic connector 170 may be used to send video signal to an external monitor. In yet another embodiment, the electrical connector 170 may have an external device, such as a wireless adapter, should a wireless system not already be included within present invention, as it may be in one embodiment, attached to it to wirelessly send data from the present invention to an external receiving device, for example a monitor, or send and receive data wirelessly to and/or from, for example, a computer or other computing devices.

As mentioned previously, there is also attached to the electronic board 190 a switch 180 for turning on and off the present device. In some embodiments, the switch 180 would allow for power from the battery 195, shown in FIG. 8B, to pass to the electronic board 190.

There is also attached to the electronic board 190, such as to electronic components located at either/or electronic component areas 146 and 147, a series of switches 145 for control of the present invention, as shown in FIG. 11. In this embodiment there are three such switches 145 for controlling the present invention, but the number of switches 145, for example 1 to 10 switches, may be present on this device depending the number of controls required for different embodiments of the present invention. One example of what a switch 145 may control is image capture from the camera. Another example of what a switch may be used for is sending data, such as still images, from a memory source within this device, to an external source, for example a computer. Yet another example of what a switch may be used for is to control the illumination within the present invention. As previously mentioned, there is a plurality of means for the switches to control, and the number of controls on embodiments of this invention will be relative to such needs.

Additionally attached to the electronic board 190, such as to electronic components located at either/or electronic component areas 146 and 147, is a switch 140 for controlling the rotation of the motor which then controls the rotation of the catheter piece. In one embodiment, the switch 140 may be configured to have one of three positions whereby there is a neutral position in the middle, for example, and a position on either side on the neutral position for rotating the motor either clockwise or counter-clockwise as would be determined by the user's input.

Another attachment to the electronic board 190, and where desired to electronic components located at either/or electronic component areas 146 and 147, are a series of connectors 199. These connectors 199 may serve a variety of functions, including for the control of the motors 157 or 156, the camera 122, the lighting 126, and the monitor 130. In another embodiment, the connectors are linked to a sensor located at the distal tip 120 of the catheter.

As shown if FIG. 8J, the portable diagnostic imaging system 100 has a connector to connect and detach the probe piece 111 of the device 100 from the hand piece 112 of the device 100. In one embodiment, the purpose of attaching and detaching the probe piece 111 of the device 100 from the hand piece 112 of the device 100 is to change the probe piece 111 from one embodiment of the probe piece 111 to another as would be the case where the two of more different probe pieces 111 have different functionality as required by the practitioner. In another embodiment of FIG. 8J, the purpose of detaching the probe piece 111 of the device 100 from the hand piece 112 is for the sterility requirements that the practitioner must follow, e.g., for a medical application. For example, should the practitioner require to use the device 100 with two of more patients, the practitioner would be required to dispose of the probe piece 111, and attach a new sterile probe piece 111 to hand piece 112.

In another embodiment of the current device 100, the monitor 113 may also be detachable from the hand piece 114 as shown in FIG. 8K. The functionality of detaching the monitor 113 from the hand piece 114 is to aid the practitioner with the viewing of the camera in a different location. In this case, the monitor 113 would be wirelessly connected to the hand piece 114 to allow video signals to be sent from the electronics within the hand piece 114 to the monitor 113.

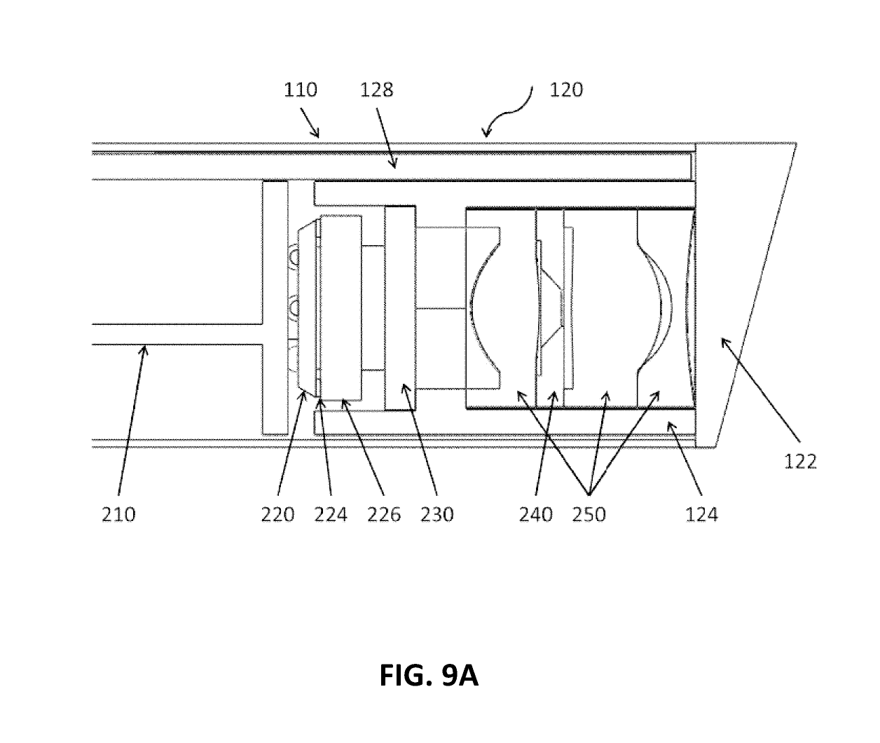

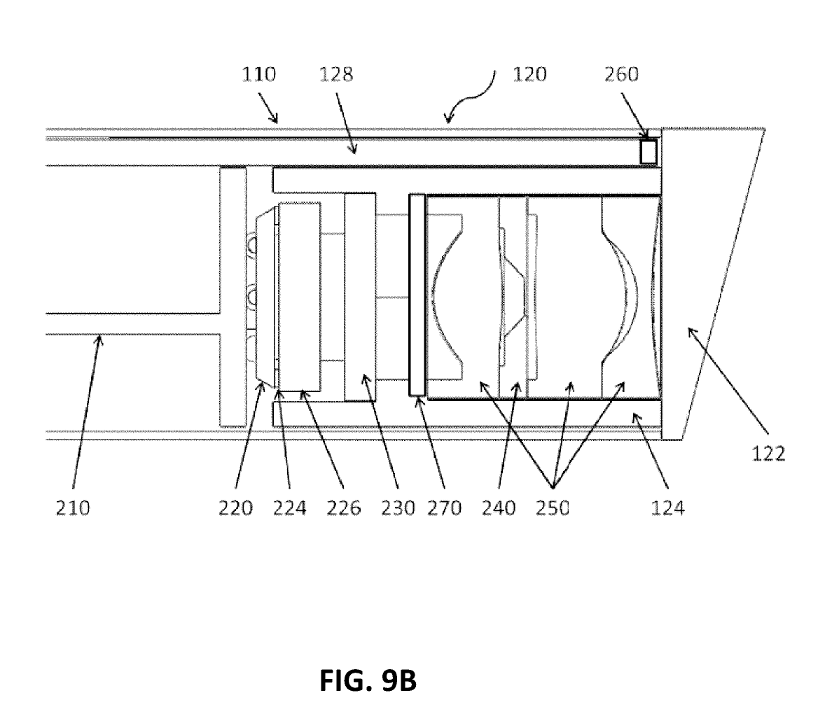

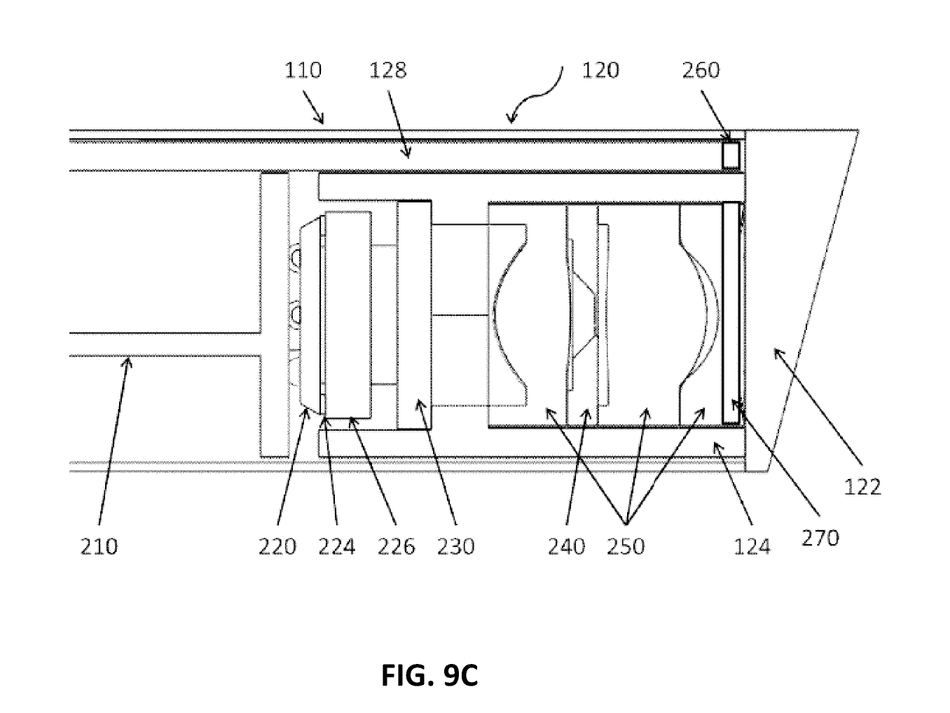

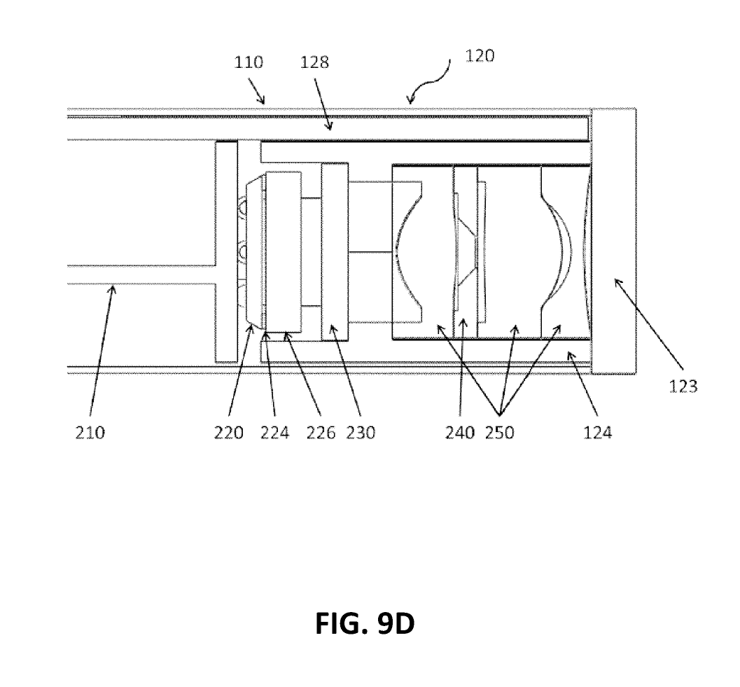

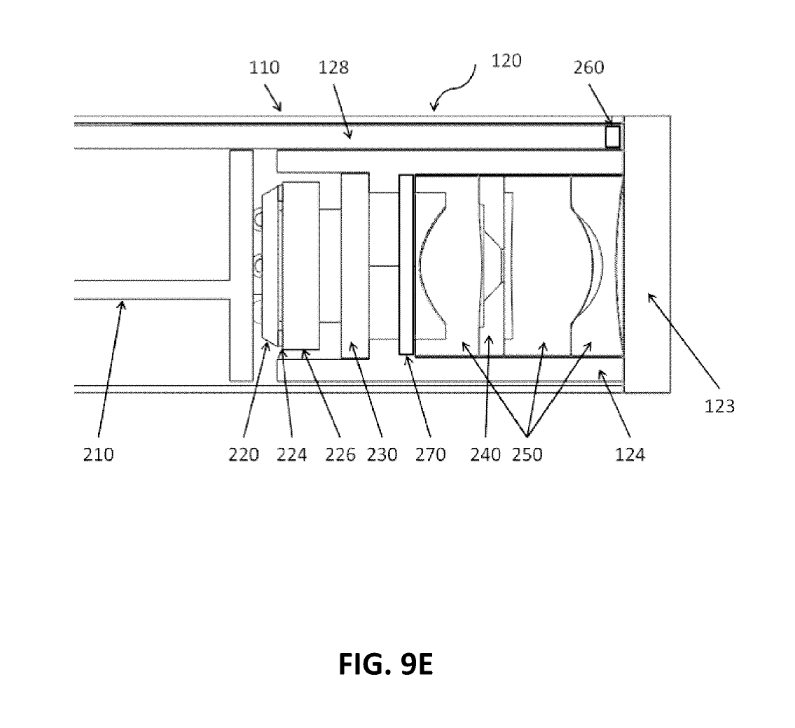

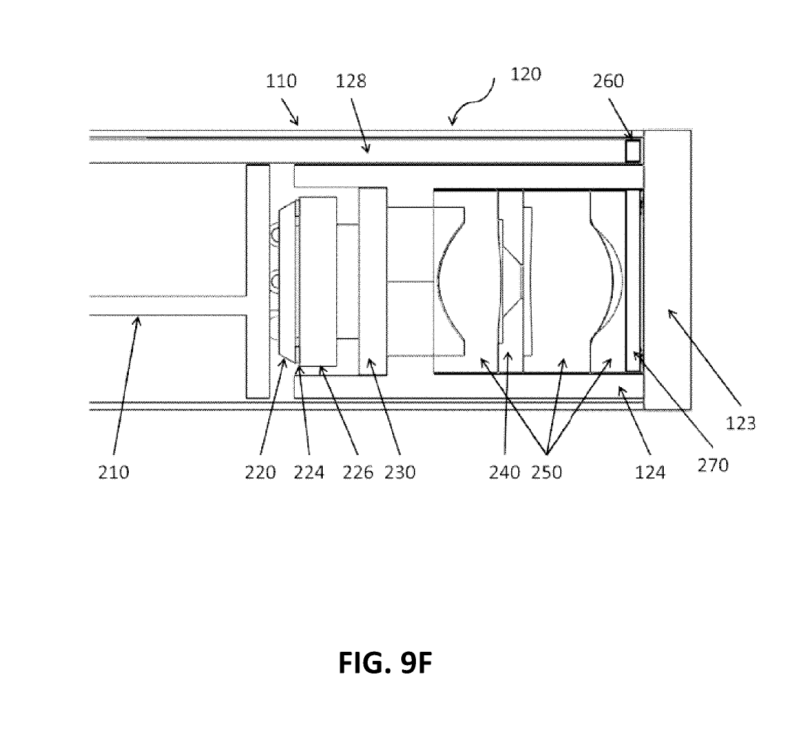

FIG. 9A shows a section view of the distal tip 120 of the probe piece 111. Shown in FIG. 9A are the necessary components that make up a camera and lighting module to produce an image that can be displayed on a monitor. The camera and lighting module as described allow viewing off-axis, and therefore make up an off-axis viewing module, as explained in greater detail below. A prism lens 122 covers the end of the elongated member 110 of the probe piece 111. The purpose of the prism lens 122 is to allow for imaging at angle to the axis of the probe piece, for example, 30 degrees. Proximal to the prism lens, in one embodiment, is shown a camera housing 124. Contained within this housing 124 is a series of lenses 250, an aperture 240, filters 230 and 226 and a CMOS imaging chip 220 that is attached to filter 226 by adhesive 224. In other embodiments of the camera, there may be more or less components as required to produce a different image. In addition, the chip 220 is mechanically and electrically attached to a circuit board 210 that transmits signals between the chip 220 and the electronics within the hand piece of the present invention. Also located within the distal tip 120 of the catheter piece is an integrated illuminator 128. In one embodiment, the integrated illuminator may be a fiberoptic bundle connected to an LED or other light source that is powered from the battery within the hand piece. In another embodiment, the integrated illuminator 128 may be a made from a light piping material such as a plastic or light transmitting hard resin or light transmitting liquid or air, all of which would be connected to an LED or other light source within the hand piece 114, as mentioned previously.

In another embodiment, of the components within the distal tip 120 as shown in FIG. 9D, a cover glass 123, is located in place of the prism lens 122 of FIG. 9A. In this case, a cover glass 123 allows the viewing of an image that is directly in from of the sensor chip 224. This configuration is an example of an “on-axis” imaging module.

One challenge with an integrated illuminator 128 and a camera being mechanically located behind a prism 122 is that stray or unintended light from the integrated illuminator 128 or other source may interfere with the camera, thereby producing sub-optimum image. To address this issue, a visualization module may include a filtering system. FIG. 9B is one embodiment of a filtering system for controlling the incidence of light form the integrated illuminator 128 or other source of light, into the chip 220. Filter 260 is polarized opposite to filter 270 so that unintended light, particularly from the integrated illuminator 128 contained within the distal tip 120 of the catheter piece is less likely to enter the camera.

In another embodiment of the filtering means, as shown in FIG. 9C, the polarizing filter 270 is located distal to the lenses 250 contained within the camera housing 124, but proximal to the prism lens.

FIGS. 9E and 9F, are embodiments of a filtering system for controlling the incidence of light form the integrated illuminator 128 or other source of light into the sensor chip 220 as previously described and shown in FIGS. 9B and 9C, with the exception that the filters as shown in FIGS. 9E and 9F, are proximal to a cover glass 123 rather than a prism lens 122 as shown in FIGS. 9B and 9C.

With reference now to FIGS. 10A-10D, there is shown an endways view of several embodiments for the mechanical arrangement of components located at the distal end 300 of a probe piece of device. As shown in FIG. 10A, an endways view of the probe-piece wall 310 has located eccentrically within its inner perimeter, a camera housing 340, camera lens and visualization sensor 330. In addition, an endways view of an integrated illuminator 320, such as the end of a fiber optic bundle, is located in the space between the camera housing 340 and inner perimeter of the probe piece wall 310. The integrated illuminator 320 has a crescent configuration so as to conform to the camera housing structure.

FIG. 10B illustrates the end of a probe piece that is analogous to that shown in FIG. 10A. In FIG. 10B, a non-visualization sensor (e.g., a pressure sensor) 390 is located on one side of the probe piece and a port 391 is located on the opposite side of the probe piece. Port 391 may be in operable connection to a lumen running at least part of the length of the probe piece, and may serve a variety of functions, including those described above, such as delivery of an active agent, etc.

Another embodiment, for the mechanical arrangement of components located at the distal end 300 of the device, is shown in FIG. 10C. An endways view of the probe piece wall 310 has located concentrically within its inner perimeter, a camera housing 340 and camera lens and visualization sensor 330. In addition, an endways view of an integrated illuminator 350, such as the end of a fiber optic bundle, is located in the space between the camera housing 340 and inner perimeter of the probe piece wall 310.

FIG. 10D illustrates the end of a probe piece that is analogous to that shown in FIG. 3C. In FIG. 10D, a non-visualization sensor (e.g., a pressure sensor) 390 is located on one side of the probe piece and a port 391 is located on the opposite side of the probe piece. Port 391 may be in operable connection to a lumen running at least part of the length of the probe piece, and may serve a variety of functions, including those described above, such as delivery of an active agent, etc.

Data transfer from the sensor to a control module in the hand piece of the device may be accomplished using any convenient approach. In certain embodiments, transferring information from sensor 390 to the electronics within the hand piece is accomplished by a connection to the electronic board 190 at a point 392 via wires 394 that are passed through the probe piece from the sensor 390 into the hand piece, as shown in FIG. 10E. FIG. 10F illustrates one embodiment of a connection from a port 391, located at the distal end of the probe piece, to a port 398 in the hand piece via an open conduit 396, for example a tube, that passes between the ports, 391 and 398, and through the inside of the probe piece.

With reference now to FIGS. 11A-11F, there is shown several different embodiments configured to maintain sterility of the hand piece. As illustrated in FIGS. 11A to 11F, there is a sterile sheath (or bag), 400 or 404, that is sealably connected to the probe piece 111 at a location 460 circumferential to the probe piece 111. The sheath 400 or 404 includes a sheath piece 450. The sheath may also include one or more additional components, such as a clear monitor cover 420 and/or or a flexible boot 430. The sheath 400 or 404 is wrapped over an embodiment of the hand piece 112 (FIGS. 11C and 11D), 102 (FIG. 11E), 104 (FIG. 11F), via an opening 440 in the hand piece portion of the sheath 450. Additionally, a seal is provided for sealing the sheath piece 450 at the opening 440 around an embodiment of the hand piece 112, 102, 104; for example, folding over the sheath piece 450 at the opening 440 and sealing it with tape or another method.

As mentioned above, and as shown in FIGS. 11A and 11C, an embodiment of the sheath 400 may have connected and sealed to it a rigid and clear monitor cover 420 and a flexible boot 430. The purpose of the monitor cover 420 is to allow for the functionality of the monitor means of the hand piece 112, while maintaining the sealability of the sheath 400. The monitor cover 420 may be comprised of a clear plastic, for example, that has the mechanical features to snap over the monitor means; the purpose of which is to allow for a clear view of the monitor for the practitioner of the present invention. The flexible boot 430 may be comprised of rubber, for example, that has the mechanical features to snap over the control elements, for example switches, of the hand piece 112, while maintaining the sealability of the sheath 400. With reference to FIG. 11D, the hand piece sheath portion 450 may then be sealed over the hand piece 112 at a location 440 as described previously.

In another embodiment of the sheath 404, as shown in FIG. 11B, there is connected and sealed to it a flexible boot 430 as mentioned in the above embodiment, but without a monitor cover 420, FIG. 11A. The purpose of this embodiment of the sheath 404 is to be able to seal a hand piece 102, FIG. 11E, that has no monitor attached to it. In this case, there may be an attachment structure 480 located on the hand piece 102, where the monitor means may be attached and/or removed as required for use by the practitioner or the present invention.

In another embodiment of the sheath means 404, as shown in FIG. 11F, there is connected and sealed to the sheath piece 450 a flexible boot 430 as mentioned in the latter embodiment and without a monitor cover 420, FIG. 11A, for the purpose of sealing a hand piece 104 that has no feature 480, FIG. 11E, where the monitor may mount, located on the hand piece at a location 470, FIG. 11F.

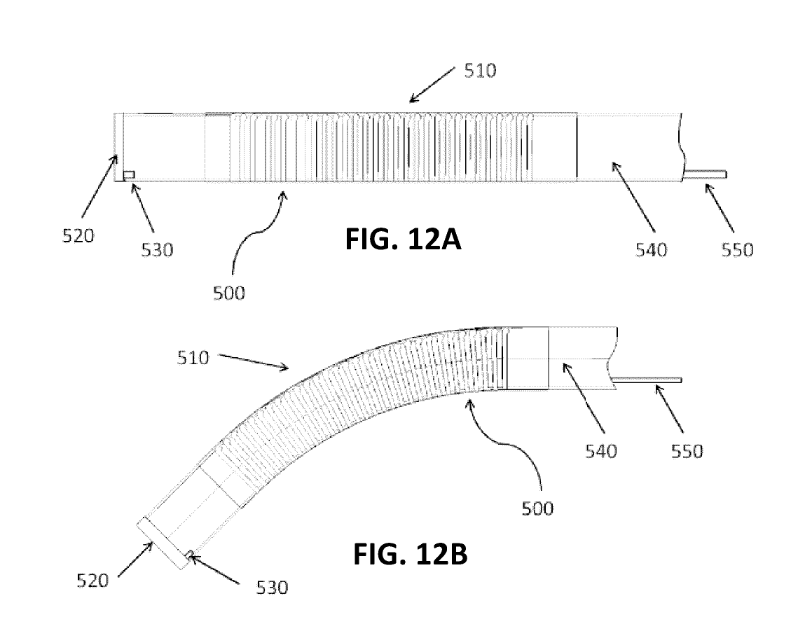

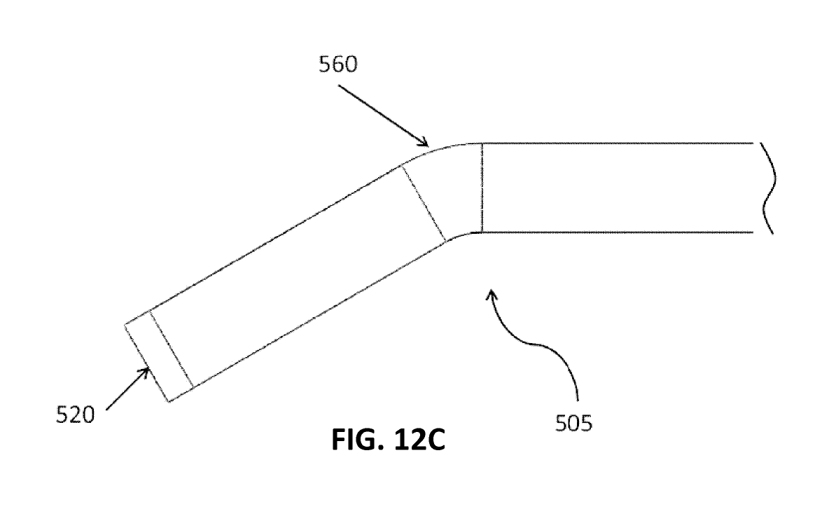

In one or more embodiments of the current invention it may be desirable to have the camera viewing in one or more directions, for example at an angle from the axis of the catheter piece, other than those directions that may be attained through the rotation of the catheter piece. The direction that the camera shall view may be controllable or fixed. With reference now to FIGS. 12A-12B, there is shown one embodiment for a flexible and controllable portion 500 of the probe piece. In this embodiment, a control cable 550, for example a twisted wire or rod, is connected at a distal location 530 to and within the tubular probe piece portion 540, and behind a distal lens 520. The control cable 550 joins to a control, for example a mechanical switch, within the hand piece, where it may be actuated, for example pulled toward the proximal end of the device. The actuation of the control cable, in this method, would cause the flexible portion 500 of the probe piece to bend as shown in FIG. 12B. The flexible portion 500 of the probe piece may then be returned to the position as shown in FIG. 12A, for example, by a spring means, or possibly by the actuation of the control cable 550 towards the distal end of the device.

The flexible portion 500 of the probe piece may be constructed in such a way as to allow for flexion of this portion of the probe piece, in one or more directions. The embodiment as shown in FIGS. 12A-12B shows one example of how to create the flexible portion 500 of the probe piece, by having a series of cut-outs covered with a hydrophobic tube 510. In this case the flexible portion 500 is configured to flex in one direction, that being shown in FIG. 12B. In addition, the purpose of the hydrophobic tubing surrounding the cut-outs 510 is to prevent material ingress into the probe piece, for example water, while allowing for the flexion of the flexible portion 500. Depending on the number and orientation of the cut-outs, this flexible portion 500 may be flexible in a plurality of directions and degrees, and may be controlled by a concomitant number of control cables connected to switches or other mechanical controls within the hand piece.

Another embodiment for the viewing of the camera at an angle, for example 30 degrees from the central axis of the catheter piece, is shown in FIG. 12C. In this case, there is an angle formed at a bend 560 in this portion of the catheter piece 505 which terminates at the proximal end of a lens 520 at the distal tip of the catheter piece. The bend 560 in this portion of the catheter piece 505 may be rigid, such as the case of a bent steel tube, or flexible, as would be the case, for example, of a formed flexible plastic tube. In the case where the formed bend 560 is flexible, there may be a spring inside, such as a NITINOL™ wire, that is configured to provide for the temporary bending of this portion 505 into a straight position, aligned with the central axis of the catheter piece, by the practitioner, and when released would bend back to the formed position.

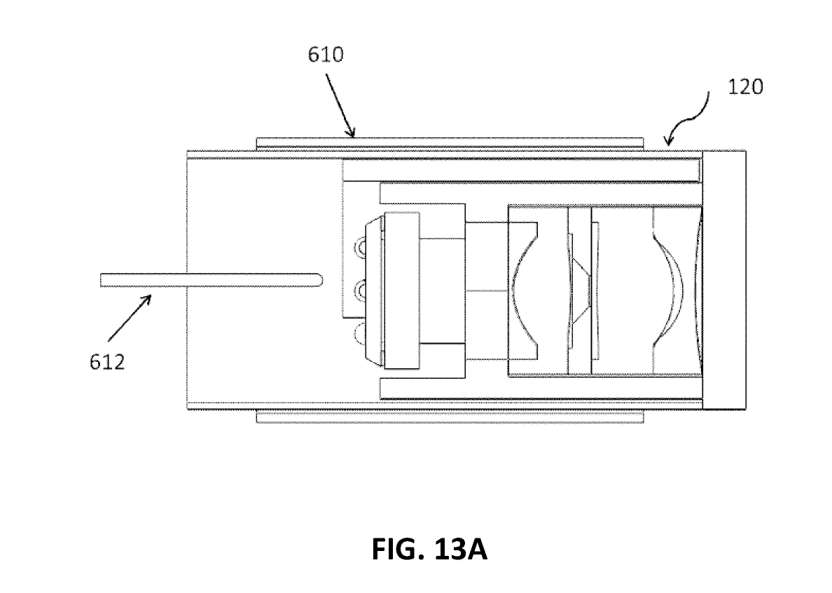

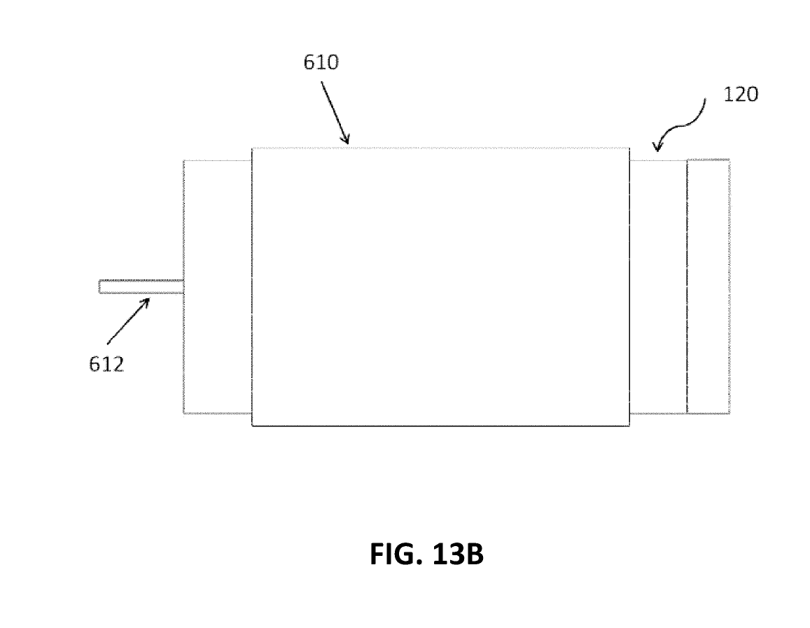

In cases where the practitioner of the present invention is required to diagnose, for example a tissue, it may be required of the practitioner to retrieve a portion of the material under diagnosis. With reference now to FIGS. 13A-13B, there is shown one embodiment of a controllable low-profile biopsy tool. FIG. 13A shows a section view of one embodiment of the distal tip 120 of the probe piece. FIG. 13B shows an external side view of one embodiment of the distal tip 120 of the probe piece. In this case, there is a low-profile biopsy tool that includes a cutting piece 610 and a control piece 612. Cutting piece 610 is concentrically disposed about the distal end of the probe piece 120, and configured to be moved relative to the distal end of the probe piece 120 in a manner sufficient to engage tissue. The control piece 612, for example a rod, may be attached to the cutting piece 610, and it may extend to the hand piece where is would be actuated by a mechanical means.

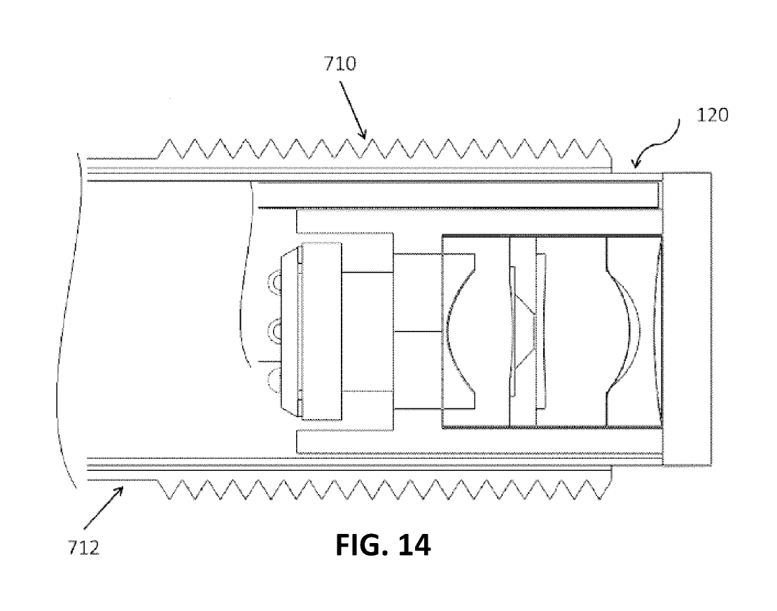

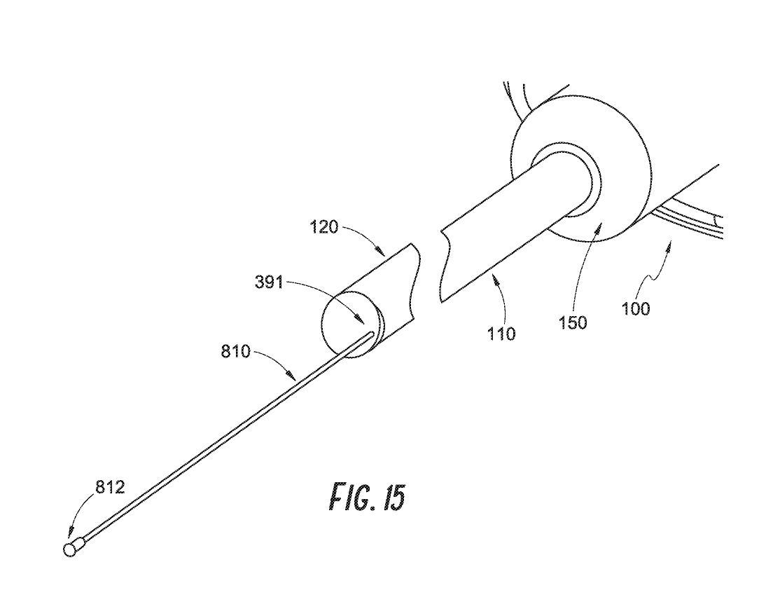

There may be cases where the practitioner of the present invention is required to scrape or cut material, for example a tissue. With reference now to FIG. 14, there is shown one embodiment of a cutting or scraping tool. This figure shows a section view of one embodiment of the distal tip 120 of the probe piece. In this case, there is a low-profile cutting or scraping tool that includes a cutting piece 710 and a control piece 712, and is concentrically disposed about the distal end of the probe piece 120. This tool may be configured to be moved relative to the distal end of the catheter piece 120 in a manner sufficient to engage material, for example tissue. In another embodiment, this tool may be configured to be rotated circumferentially to the distal end of the catheter piece 120 in a manner sufficient to engage material, for example tissue. In yet another embodiment, this tool may be fixed at the distal end of the catheter piece 120. The control piece 712, for example a tube or rod, may be attached to the cutting piece 710, and it may extend to the hand piece where is would be actuated by a mechanical means should that be necessary for the particular embodiment of the tool.

There may be cases where the practitioner of the present invention is required to deploy one or more sensors in or near or around a material, for example a tissue. Such may be the case in a diagnosis of a material, for example a tissue, where monitoring the material in question requires a continuous sensing and also requires the removal of the visualization means of the present invention from, for example a patient under diagnosis. With reference now to FIG. 8, there is shown one embodiment of a deployable sensor 812 incorporated into a device of the present invention 100 by a wired connection 810. Alternatively, a wireless communication module may be employed instead of wired connection 810. As illustrated, the wired connection passes through a port 391, as shown in FIGS. 10B and 10D where it then passes through the distal tip 120 of the probe piece and the elongated member 110 of the probe piece and the connector 150 of the probe piece. The wired connection 810 then connects to the electronics board within the hand piece where its output may be processed. This processed output may be displayed on a monitor and/or recorded to a memory chip on the electronics board, for example. The wired connection 810 may have sufficient slack, for example extra wire length, so as to allow the sensor to be located at some distance, for example 200 mm, from the visualization sensor. In one embodiment, the deployable sensor 812 may have mechanical features that aid in the deployment of the sensor, for example a hook or a spike or a barb.

As mentioned previously, there may be a wireless deployment of the sensor 812. In this case, the sensor 812 would wirelessly connect to the electronics board within the handle where its output would be processed. Any convenient wireless communication protocol may be employed. This processed output may be displayed on a monitoring means and/or recorded to a memory chip on the electronics board, for example.

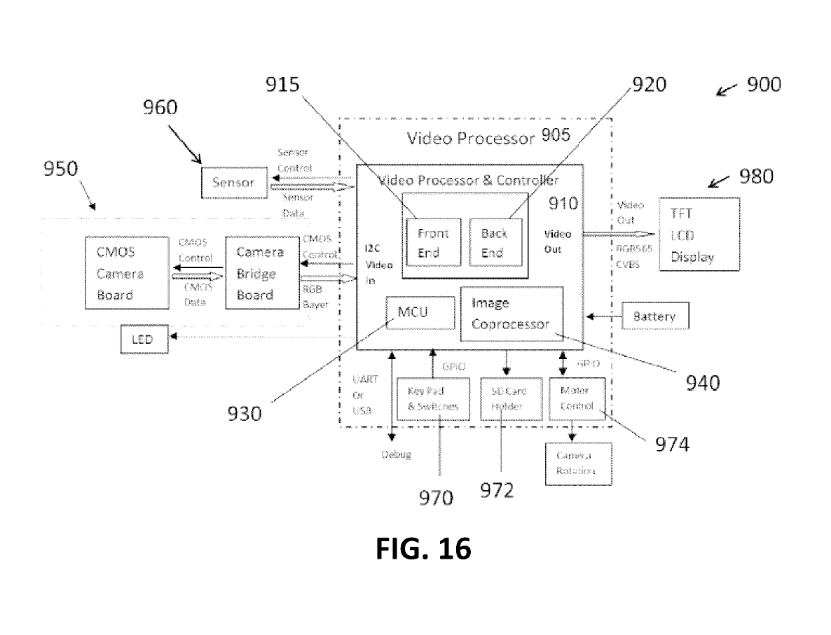

FIG. 16 illustrates a functional block diagram of a system 900 including a video processor module 905, according to one embodiment. Video processor module 905 includes a processor/controller module 910 which is in communication with sensor module 960, camera module 950, and display 980. Processor/controller module 910 comprises front end module 915, back end module 920, microcontroller 930, and image coprocessing module 940. Image coprocessing module 940 includes, for example, stereoscopic image module and performs the previously described functions and operations of the stereoscopic image module.

Camera module 950 may include a single visualization sensor, or two or more distinct visualization sensors which provide image data. Front end module 915 includes circuitry for receiving the image data from the camera module 950. The image data received from camera module 950 is processed by stereoscopic image module (i.e., by image coprocessing module 940) to provide stereoscopic image data. For example, as previously described, the image data from each distinct visualization sensor may be warped to correct image distortion, and fused to construct a single stereo image taking into account three-dimensional depth information. Back end module 920 includes circuitry for sending the stereoscopic image data to display 980. Display 980 displays a three-dimensional view of the image data for the user to see.

Video processor module 905 may be electrically coupled with camera module 950 via an I2C bus, for example, with camera module 950 configured as the slave and microcontroller 930 as the master. Microcontroller 930 may be configured to send camera control data to the camera module 950. The camera control data may comprise information requests (e.g., for information relating to testing/debugging, for calibration data, etc.) or provide commands for controlling the camera module 950 (e.g., controlling the two or more distinct visualization sensors, etc.).

Sensor module 960 may include one or more sensors and/or tools previously described. The one or more sensors and/or tools implemented may provide sensor data related to their specific function and application. The sensor data is received by processor/controller module 910 and may be used in a variety of ways depending on the specific function of the sensor(s) and/or tool(s) and their application. For instance, sensor data may be used by processor/controller module 910 to provide information to a user (e.g. parameter data, calibration data, measurement readings, warnings, etc., to be displayed on display 980 or to illuminate one or more LEDs), to account for feedback signals for more accurate control of a specific sensor(s) and/or tool(s), to store in memory, to further process into additional related information, etc. Microcontroller 930 may also control the sensor module 960 via the I2C bus or General Purpose Input/Output (GPIO) interface by sending sensor control data (e.g., to control and/or calibrate the specific sensors and/or tools implemented).

Processor/controller module 910 further comprises various modules for interfacing with external devices and peripherals. For example, as shown in FIG. 9, processor control module includes a key pad and switches circuitry 970 for receiving input signals from the user key pad and switches on the device; SO card holder circuitry 972 for sending/receiving data stored in memory devices, and motor control circuitry 97 4 for controlling the camera rotation. Microcontroller 930 may be configured with, for example, a GPIO to communicate with the various circuitry. Furthermore, the video processor module 905 may include a communication interface for implementing testing or debugging procedures—e.g., UART, USB, etc.

### EXPERIMENTAL EXAMPLES

The following examples are offered by way of illustration and not by way of limitation.

A hand-held minimally dimensioned diagnostic device having integrated distal end visualization was constructed as follows. The device consisted of an outer SLA shell in the form of a hand-held unit housing batteries, a 3.5″ monitor, a control board, and wires that connect to 2 LEOS and a visualization module at the distal tip of a steel 4 mm hypodermic tube that was connected to the handle. The tubing was bent about an inch back from the distal tip to about 30 degrees. A manual wheel was provided on the hand-piece connected to the tube, and when actuated, rotated the tube 180 degrees in each direction. Considering a field of view for the camera of roughly 120 degrees (diagonal), the rotation of the tube allowed the camera to view at least a full hemisphere of space. The visualization module at the 4 mm outer diameter distal tip of the hypodermic tube included an Omnivision 6920 QVGA imaging chip (Santa Clara, Calif.), a series of lenses, an aperture, IR filter and a cover-glass within a small steel housing. In addition, LEOS were placed behind the flat cover-glass, but distal to the aperture. Thus due to the configuration of camera lens and lighting, there is little incidence of stray light affecting the image. In the constructed device, the signal from the powered camera goes through a series of electronic components where it is processed in a manner useful for the control board, and wires send the data to the control board where it is then displayed on the monitor. The monitor also rotates. QVGA resolution was observed for the image displayed on the 3.5 inch monitor.

### Embodiments of RF Tissue Modulation Devices

As summarized above, RF tissue modulation devices of the invention may include an elongated member and a hand-held control unit (such as an RF probe and hand-held control unit described further below). For example, the elongated member may be operably coupled to the hand-held device at a proximal end of the elongated member. In other aspects of the invention, RF tissue modulation devices may include an elongated member and an adapter configured to be independently removably coupled to a medical device (e.g., a visualization device). In some instances, the elongated member removably couples to the medical device. It should also be understood, that that in some instances, the elongated member may be affixed to the medical device, adapter, and/or hand-held control unit. Furthermore, it should be understood that the term RF tissue modulation devices is used herein to refer generally to cumulative devices (e.g., RF probe and hand-held device; or, RF probe, adapter, and medical device), and in some instances to refer to each of the individual or combination of component devices (e.g., RF probe, or adapter, or RF probe and adapter, etc.).

In addition to the above two components, devices of certain embodiments of the invention may include an RF energy source that is configured to generate a plasma at the plasma generator of the elongated member (e.g., RF probe) for a therapeutic duration, e.g., as described above. The RF energy source may include a number of distinct components, such as but not limited to: an electrical energy source, voltage converter, charge accumulator, and RF signal generator. In certain instances, the devices may also include an adaptor, as described in greater detail below. The various components of the RF energy source may be present in one of the handheld control unit or adaptor (when present) or distributed among the various components of the device, e.g., the hand held control unit, adaptor and/or RF probe.

### RF Probe

The RF probe is an elongated member that is configured to be operably coupled to a hand-held control unit. With respect to the elongated member, this component has a length that is 1.5 times or longer than its width, such as 2 times or longer than its width, including 5 or even 10 times or longer than its width, e.g., 20 times longer than its width, 30 times longer than its width, or longer. The length of the elongated member may vary, and in some instances ranges from 5 cm to 20 cm, such as 7.5 cm to 15 cm and including 10 to 12 cm. The elongated member may have the same outer cross sectional dimensions (e.g., diameter) along its entire length. Alternatively, the cross sectional diameter may vary along the length of the elongated member.

As described above, in some instances, at least the distal end region of the elongated member of the device is dimensioned to pass through a Cambin's triangle. By distal end region is meant a length of the elongated member starting at the distal end of 1 cm or longer, such as 3 cm or longer, including 5 cm or longer, where the elongated member may have the same outer diameter along its entire length. The Cambin's triangle (also known in the art as the Pambin's triangle) is an anatomical spinal structure bounded by an exiting nerve root and a traversing nerve root and a disc. The exiting root is the root that leaves the spinal canal just cephalad (above) the disc, and the traversing root is the root that leaves the spinal canal just caudad (below) the disc. Where the distal end of the elongated member is dimensioned to pass through a Cambin's triangle, at least the distal end of the device has a longest cross sectional dimension that is 10 mm or less, such as 8 mm or less and including 7 mm or less. In some instances, the devices include an elongated member that has an outer diameter at least in its distal end region that is 5.0 mm or less, such as 4.0 mm or less, including 3.0 mm or less.

The elongated members of the subject RF tissue modulation devices have a proximal end and a distal end. The term “proximal end”, as used herein, refers to the end of the elongated member that is nearer the user (such as a physician operating the device in a tissue modification procedure), and the term “distal end”, as used herein, refers to the end of the elongated member that is nearer the internal target tissue of the subject during use. The proximal end is also the end that is operably coupled to the hand-held control unit of the device (described in greater detail below). The elongated member is, in some instances, a structure of sufficient rigidity to allow the distal end to be pushed through tissue when sufficient force is applied to the proximal end of the elongate member. As such, in these embodiments the elongated member is not pliant or flexible, at least not to any significant extent.

As summarized above, some embodiments of the RF tissue modulation devices include a plasma generator integrated at the distal end of the elongated member, such that the plasma generator is integrated with the elongated member. As the plasma generator is integrated at the distal end of the device, it cannot entirely be removed from the remainder of the device without significantly compromising the structure and functionality of the device. While the plasma generator cannot entirely be removed from the device without compromising the structure and functionality of the device, components of the plasma generator may be removable and replaceable. For example, an RF electrode of a plasma generator according to some embodiments may be configured such that a wire component of the plasma generator may be replaceable while the remainder of the plasma generator is not. Accordingly, the devices of the present invention are distinguished from devices which include a “working channel” through which a separate autonomous plasma generator device, such as autonomous RF electrode device, is passed through. In contrast to such devices, since the plasma generator of the present device is integrated at the distal end, it is not a separate device from the elongated member that is merely present in a working channel of the elongated member and which can be removed from the working channel of such an elongated member without structurally compromising the elongated member in any way. The plasma generator may be integrated with the distal end of the elongated member by a variety of different configurations. Integrated configurations include configurations where the plasma generator is fixed relative to the distal end of the elongated member, as well as configurations where the plasma generator is movable to some extent relative to the distal end of the elongated member may be employed in devices of the invention. Specific configurations of interest are further described below in connection with the figures. As the plasma generator is a distal end integrated plasma generator, it is located at or near the distal end of the elongated member. Accordingly, it is positioned at 30 mm or closer to the distal end, such as at 20 mm or closer to the distal end, including at 10 mm or closer to the distal end. In some instances, the plasma generator is located at the distal end of the elongated member.

The plasma generator may be configured in a variety of ways for a controllable delivery of RF energy. The plasma generator may include one or more RF electrodes positioned at the distal end of the elongated member. RF electrodes are devices for the delivery of radiofrequency (RF). In some instances, the RF electrodes are electrical conductors, such as a metal wire, or other conductive member, and can be dimensioned to access an intervertebral disc space for example.

RF electrodes may be shaped in a variety of different formats, such as circular, square, rectangular, oval, etc. The dimensions of such electrodes may vary, where in some embodiments the RF electrode has a longest cross sectional dimension that is 7 mm or less, 6 mm or less 5 mm or less, 4 mm or less, 3 mm or less or event 2 mm or less, as desired. Where the RF electrode includes a wire, the diameter of the wire in such embodiments may be 180 m, such as 150 μm or less, such as 130 μm or less, such as 100 μm or less, such as 80 μm or less.

Various RF electrode configurations for use in tissue modification devices are described in U.S. Pat. Nos. 7,449,019; 7,137,981; 6,997,941; 6,837,887; 6,241,727; 6,112,123; 6,607,529; 5,334, 183; in Provisional Application Ser. No. 61/082,774; in U.S. patent application Ser. No. 12/422,176; and in International Patent Application Serial No. US09/51446; the disclosures of which are herein incorporated by reference. RF electrode systems or components thereof may be adapted for use in devices of the present invention (when coupled with guidance provided by the present specification) and, as such, the disclosures of the RF electrode configurations in these patents are herein incorporated by reference. Specific RF electrode configurations of interest are further described in connection with the figures, below.

In some aspects of the invention, the plasma generator is configured to generate a plasma between two or more RF electrodes. In some instances, one or more of the RF electrodes is a grounded conductive member, wherein a plasma is generated between an RF electrode and a grounded RF electrode (e.g., grounded conductive member, such as grounded outer surface of the elongated member, etc.). The RF electrodes are provided with the necessary power and voltage to generate a plasma between the electrodes. In some instances, the plasma is only generated when the plasma generator is partially or fully submerged in saline solution such that only a portion of the plasma field is exposed to the patient. The surrounding saline solution provides a conductive path between the electrodes as well as the sodium ions required to produce the plasma. The saline solution may also help to disperse the thermal effects generated by the plasma field. Such limited exposure may also help to confine the treated region to the surface surrounding tissue. In some instances, the plasma may be generated in other mediums, such as air, blood, tissue, etc.

RF electrodes may be positioned in a variety of ways at the distal end of the elongated member. For example, one or more RF electrodes may be positioned on the elongated member, extending from the elongated member, and/or positioned within the elongated member. In some instances, the plasma generator is configured to produce a plasma between an RF electrode positioned inside of the distal end of the elongated member and an outer surface of the elongated member. In some instances, the plasma generator may be configured to produce a plasma between an RF electrode positioned substantially at a tip of the elongated member and the outer surface of the elongated member. In this way, the tip of the elongated member may correspond approximately to the target tissue site.

The position of the RF electrodes may depend on specific application and design considerations (e.g., field of view of the user holding the device, and/or positioning of other components in the elongated member (e.g., visualization sensor, illuminator, etc.). For example, in some instances, the elongated member may include a distal end integrated visualization sensor in addition to the plasma generator, and the hand-held device further include a monitor, such as described in further detail below.

The elongated member may also include an opening positioned at the distal end of the elongated member. The opening may be of a variety of shapes—e.g., oval, circular, rectangular, open-ended, etc.). The size of the opening may vary depending on particular application and design considerations. Example opening sizes may include, for example, 20 mm or less, such as 10 mm or less and including 5 mm or less, e.g., 2.5 mm or less. In some embodiments, the elongated member may include an opening positioned over a conductive member acting as an RF electrode positioned within the distal end of the elongated member. In another embodiment, the elongated member may include a conductive member acting as an RF electrode positioned within or near the opening at the distal end of the elongated member.

In some instances, the elongated member includes one or more insulators coupled to one or more RF electrodes. The insulator may be of a variety of materials, such as ceramic, or any other insulative material. The insulators may be used to maintain the RF electrodes in position. For example, an insulator may be positioned in the elongated member to maintain an RF electrode (e.g., conductive member such as a small metal wire or plate) within the elongated member. Multiple insulators may be positioned within or on the elongated member to maintain one or more RF electrodes within or near an opening at the distal end of the elongated member.

In some aspects of the invention, the plasma generator receives an RF signal generated by an RF energy source. The plasma generator is supplied with current and the voltage signal driving the current to the plasma generator may be definable as a sine, square, saw-tooth, triangle, pulse, non-standard, complex, or irregular waveform, or the like, with a well-defined operating frequency. For example, the operating frequency can range from 1 KHz to 50 MHz, such as from 100 KHz to 25 MHz, and including from 250 KHz to 10 MHz. Furthermore, the operating frequency can be modulated by a modulation waveform. By “modulated” is meant attenuated in amplitude by a second waveform, such as a periodic signal waveform. The modulation waveform may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 70 Hz. Thus, in some instances, the plasma generator receives a high voltage modulated RF signal and generates a plasma.

In some aspects of the invention, an RF line may couple one or more RF electrodes described above to an RF energy source. The RF line may be made of any conductive material, such as metal, metal alloys, etc. The RF line electrically couples the plasma generator to the RF energy source at another location of the device, such as a proximal end location. Such proximal end location may include, for example, a hand-held control unit or adapter as described in further detail below. The RF line may be positioned, for example, within or along the elongated member to couple the proximal end RF energy source to the distal end plasma generator.

The RF tissue modulation device may be configured to deliver RF energy to the plasma generator for a therapeutic duration. The therapeutic duration may last, for example, minutes or less, such as 1 minute or less, including 30 seconds or less, such as 10 seconds or less. In some instances, the therapeutic duration may range from 1 to 2 seconds. Visualization capabilities (as developed in greater detail below), if implemented, may be available for a duration independent of the therapeutic duration. For instance, visualization capabilities may continue after RF energy treatment.

In some instances, an RF shield is positioned within the elongated member adjacent to the RF line in order to provide RF shielding for the ambient RF field generated. The RF shielding is positioned in the elongated member so as to minimize ambient RF interference and disturbances encountered by other components in the device (e.g., visualization sensors, chips, etc.). The term “adjacent to” herein is meant to include next to, surrounding, or substantially next to or surrounding. For example, RF shielding may be provided around the RF line and/or substantially around the RF electrodes. In some instances, RF shielding may be provided substantially around or near other components which require protection from ambient RF. In some instances, the RF shielding is provided between the components which require protection and the RF line (and/or RF electrodes) but not necessarily around them.

### RF Energy Source

In some aspects of the invention, embodiments of the RF tissue modulation devices include an RF energy source used to generate RF energy for delivery to the plasma generator. For example, the RF energy source may generate a high voltage modulated RF signal for delivery to the plasma generator. The RF energy source may include, for example, an electrical energy source, a voltage converter, charge accumulator, and a RF signal generator. In some instances, the voltage converter, charge accumulator, and RF signal generator operably couple the electrical energy source to a plasma generator on an elongated member (e.g., RF probe).

In some aspects of the invention, the RF energy source is included in a hand-held control unit. In some instances, the hand-held control unit may be a hand-held medical device (such as, for example, a tissue visualization device as described in U.S. application Ser. No. 12/501,336, the disclosure of which is hereby incorporated by reference) that has been configured to further include an RF energy source. In some aspects of the invention, the RF energy source is included in an adapter configured to removably couple to a hand-held device, such as a hand-held medical device, such as a tissue visualization device as described in U.S. application Ser. No. 12/501,336, the disclosure of which is hereby incorporated by reference.

The electrical energy source may include one or more power sources—e.g., one or more DC batteries. While the electrical energy source is described as being located within the hand-held control unit or adapter, in some instances, the electrical energy source may be remote from the hand-held control unit or adapter—e.g., in a battery pack configured to be electrically coupled to the hand-held control unit or adapter—e.g., via cables. However, providing the electrical energy source within the hand-held control unit or adapter allows the RF tissue modulation device to remain untethered and more portable, which may be user-friendly for the operator of the device.

The charge accumulator stores electrical energy which is later discharged when RF energy is to be delivered to the plasma generator. The charge accumulator may be, for example, one or more capacitors that charge until delivery of RF energy is activated by the user. In some instances, the charge accumulator is coupled to an electrical energy source and stores energy in one or more capacitors until RF energy is activated. To activate the RF energy, the user may engage a switch or other activation element, such as a button, key, wheel, trigger, etc., which initiates the decoupling of the charge accumulator from the electrical energy source so that it may begin discharging. The one or more capacitors may be selected to provide most the current, so that less current is required by the electrical energy source. In some instances, this configuration provides a large current in a short amount of time. Further, the one or more capacitors may be chosen, for example, to have less impedance than the internal resistance of the DC batteries.

In some instances, the charge accumulator may be configured to receive a voltage signal from a component other than the electrical energy source. For example, the charge accumulator may be coupled to the voltage converter and receive a high voltage signal which charges the charge accumulator. When RF energy is activated, the voltage converter is disconnected from the charge accumulator, for example, to provide for discharge.

In some instances, the charge accumulator may include two or more capacitor pairs which may be discharged sequentially in stages. For example, each pair of capacitors may be configured to provide a respective positive and negative voltage output. In some instance, a modulation circuit may be configured to couple to the charge accumulator and discharge the two or more capacitor pairs sequentially at a modulated rate based on a clock signal from a clock source. For example, the modulation circuit may include a demultiplexer configured to receive a count from a counter and to discharge stages of the charge accumulator based on the count. The counter may be configured to count at a rate based on the clock signal from the clock source and discharge each stage on an associated count. The modulation circuit may further include a timer coupled to the enable input of the demultiplexer, for example, to activate the discharging of the capacitor pairs when RF energy is activated. Upon completion of the timer count, the timer disables the demultiplexer so that the capacitor pairs are no longer triggered to discharge and may once again charge.

The voltage converter receives an input signal at a first voltage level and generates an output signal at a second voltage level. Voltage converters may include, for example, a DC to DC converter, transformer, etc. The voltage converter boosts the voltage level and generates a high voltage signal necessary for plasma generation. While it should be understood that a voltage boost is not necessarily required if the electrical energy source provides sufficient voltage, in typical applications, practical design considerations (e.g., weight and size) limit the batteries to a voltage level which requires further boosting.

In some embodiments, the voltage booster is configured to receive a modulated RF signal and to output a high voltage modulated RF signal. In some embodiments, the voltage converter is configured to receive a DC voltage signal from the charge accumulator and to output a high voltage signal (e.g., to an RF signal generator). In some instances, the voltage converter may further be configured to receive a clock signal from a clock source, in addition to a voltage signal, and to output a modulated high voltage signal based on the clock signal. In some instances, the voltage converter may include more than one DC to DC converter and be configured to generate a positive and negative high voltage rail with a common ground.

The RF signal generator generates an RF signal at a desired operating frequency to provide the necessary power delivery to the plasma generator. The RF signal may be in the form of, for example, a sine, square, saw-tooth, triangle, pulse, non-standard, complex, or irregular waveform, or the like, with a well-defined operating frequency. For example, the operating frequency can range from 1 KHz to 50 MHz, such as from 100 KHz to 25 MHz, and including from 250 MHz to 10 MHz. In some embodiments, the RF voltage signal is a sine wave with operating frequency 460 kHz.

In some embodiments, the RF signal generator includes an RF power amplifier and an RF clock source. The RF power amplifier receives an RF clock signal generated by the RF clock source and generates an RF signal at an operating frequency based on the RF clock signal. In some instances, the RF power amplifier may be configured to receive a voltage signal used as a bias voltage input. The bias voltage input may affect, for example, the peak voltage of the signal output by the RF power amplifier. The bias voltage signal may be received by another component such as the charge accumulator, DC to DC converter, or other voltage source. For example, in some embodiments, the RF signal generator is configured to receive a bias voltage signal from a charge accumulator, as well as receive an RF clock signal from an RF clock source, and to output an RF signal based on the bias voltage signal and RF clock signal.

In some embodiments, the RF power amplifier is configured to receive a second clock signal from a second clock source and generate a modulated RF output signal based on the second clock signal. By “modulated” it is meant that the modulation frequency comprises attenuating the amplitude of the RF signal based on the second clock signal. The modulation waveform may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 50 Hz.

In some instances, the RF power amplifier is configured to receive a modulated bias voltage signal (e.g., from another component such as a DC to DC converter, or other voltage converter), as well as an RF clock signal from an RF clock source, and to output a modulated RF signal—e.g., the RF signal is based on the RF clock signal and is modulated based on the modulated bias voltage signal. For example, the voltage converter may be coupled to a clock source and receive a clock signal and provide a high voltage modulated output signal based on the clock signal to the RF power amplifier.

In some embodiments, the RF signal generator comprises an H-bridge. In some instances, the H-bridge is coupled to an RF clock source and configured to receive an RF clock signal from the RF clock source and operate at a frequency based on the RF clock signal. For instance, the H-bridge may receive positive and negative voltage input signals and generate positive and negative voltage output signals that switch polarities at an operating frequency based on the RF clock signal.

In some instances, the RF energy source may include a bandpass filter to filter out out-of-band frequencies. Any convenient bandpass filter may be employed.

The RF energy source may also include an RF tuner in some embodiments. The RF tuner includes basic electrical elements (e.g., capacitors and inductors) which serve to tailor the output impedance of the RF energy system. The term “tailor” is intended here to have a broad interpretation, including affecting an electrical response that achieves maximum power delivery, affecting an electrical response that achieves constant power (or voltage) level under different loading conditions, affecting an electrical response that achieves different power (or voltage) levels under different loading conditions, etc. Furthermore, the elements of the RF tuner can be chosen so that the output impedance is dynamically tailored, meaning the RF tuner self-adjusts according to the load impedance encountered at the electrode tip. For instance, the elements may be selected so that the electrode has adequate voltage to develop a plasma corona when the electrode is placed in a saline solution (with saline solution grounded to return electrode), but then may self-adjust the voltage level to a lower threshold when the electrode contacts tissue (with tissue also grounded to return electrode, for example through the saline solution), thus dynamically maintaining the plasma corona at the electrode tip while minimizing the power delivered to the tissue and the thermal impact to surrounding tissue. RF tuners, when present, can provide a number of advantages. For example, delivering RF energy to target tissue through the distal tip of the electrode is challenging since RF energy experiences attenuation and reflection along the length of the conductive path from the RF energy source to the electrode tip, which can result in insertion loss. Inclusion of an RF tuner, e.g., as described above, can help to minimize and control insertion loss.

The RF tissue modulation devices may be configured to deliver RF energy from the RF energy source to the plasma generator for a therapeutic duration. The therapeutic duration may range, for example, from minutes or less, such as 1 minute or less, including 30 seconds or less, such as 10 seconds or less. In some instances, the therapeutic duration may range from 1 to 2 seconds

Furthermore, the RF tissue modulation device may be configured to recharge the charge accumulator within a minimum recharge period between plasma generation. The minimum recharge period may range, for example, from 10 minutes or less, including 5 minutes or less, such as 3 minutes or less. In some instances, the minimum recharge period ranges from 1 to 2 minutes.

### Further Embodiments of the Hand-Held Control Unit

As summarized above, the RF tissue modulation devices of the invention further include a hand-held control unit to which the elongated member is operably connected. By “operably connected” is meant that one structure is in communication (for example, mechanical, electrical, optical connection, or the like) with another structure. The hand-held control unit is located at the proximal end of the elongated structure. As the control unit is hand-held, it is configured to be held easily in the hand of an adult human. Accordingly, the hand-held control unit may have a configuration that is amenable to gripping by the human adult hand. The weight of the hand-held control unit may vary, but in some instances is 10 lbs or less, including 5 lbs or less, such 3 lbs or less. In some instances, the weight of the hand-held control may weigh 2 lbs or less, including 1 lb or less. The hand-held control unit may have any convenient configuration, such as a hand-held wand with one or more control buttons, as a hand-held gun with a trigger, etc.

In some aspects of the invention, the hand-held control unit includes the RF energy source. For example, the hand-held control unit may include an electrical energy source, a charge accumulator, voltage converter, and RF signal generator, wherein the voltage converter, charge accumulator, and RF signal generator operably couple the electrical energy source to a plasma generator on an elongated member (e.g., RF probe). In some instances, the RF energy source may additionally include a bandpass filter and/or RF tuner. In some instances, the bandpass filter and/or RF tuner are located external to the hand-held control unit—e.g., in the elongated member.

As stated before, in some instances, the hand-held control unit may be a hand-held medical device (such as, for example, a tissue visualization device as described in U.S. application Ser. No. 12/501,336, the disclosure of which is hereby incorporated by reference) that has been configured to further include an RF energy source.

### Adapter

In some aspects of the invention, an adapter is provided that includes the RF energy source. For example, in some embodiments, the adapter includes an electrical energy source, a charge accumulator, voltage converter, and a RF signal generator, wherein the voltage converter, charge accumulator, and RF signal generator operably couple the electrical energy source to a plasma generator on an elongated member (e.g., RF probe). Furthermore, in some instances, the adapter may additionally include a bandpass filter and/or RF tuner.

In some aspects of the invention, the adapter is configured to operably and removably couple to a hand-held minimally dimensioned medical device. In some embodiments, the adapter may be configured to removably couple to a minimally dimensioned visualization device (such as, for example, a tissue visualization device as described in U.S. application Ser. No. 12/501,336, the disclosure of which is hereby incorporated by reference) that has been configured to couple to the adapter. For example, the visualization device may be configured to include a removable section that removes so that the adapter may operably couple in place of the removable section. It should be understood that the adapter of the present invention may be configured to removably couple and operate with a variety of medical devices other than a visualization device.

The size of the adapter may vary depending on the particular application and design consideration (e.g., how many batteries are required, whether a transformer is included, etc.). Generally, the adapter is large enough to house the RF energy source components and yet be minimally sized to maintain the hand-held nature of the device. In some instances, the adapter is smaller than five times the size of the hand-held device, including smaller than three times the size of the device, such as smaller than two times the size of the device. For example, in some instances, the device may be smaller than the size of the hand-held device. Furthermore, the weight of the adapter may vary and depends largely on the components included within. For instance, components such as batteries and transformers may provide extra weight to the adapter. The weight of the adapter may vary, but in some instances ranges from 10 lbs or less, including 5 lbs or less, such 3 lbs or less. In some instances, the weight of the hand-held control may weigh 2 lbs or less, such as 1 lb or less, including 0.5 lbs or less.

As the adapter is removably coupled to a hand-held medical device, it is configured to maintain the hand-held nature of the device—e.g., remain amenable to gripping by the human adult hand. The adapter may vary in shape and is generally shaped to couple to the hand-held device without inhibiting or negatively affecting the use of the device by the user—e.g., inhibiting movement of the device, inhibiting field of vision for the user, etc. In some instances, the adapter is configured to removably couple to the hand-held device in a manner such that it is positioned below the hand-held device when coupled. For example, the adapter may be arc-shaped or u-shaped and positioned below the hand-held device so as to provide a space between the inner are or “u” of the adapter and the hand-held device, thus allowing the user to grip the hand-held device without the adapter obstructing the grip. In another example, the adapter is rectangularly shaped and positioned below the hand-held device when coupled—e.g., extending lengthwise downward from the device. In such case, the adapter may couple to the proximal or distal end of the device and still allow the user to grip the device. In some instances, the adapter may be configured to allow the user to grip the adapter when coupled to the device—e.g., forming a gun-shape with the device. Additionally, the adapter may be configured to include switches or other control elements, such as an activation switch to activate RF energy.

The adapter may be coupled to the hand-held device using a variety of mechanisms—e.g., hinge, magnet, Velcro, ball and socket, etc. Furthermore, the adapter may couple to the hand-held device at one or more interface locations. For example, if the adapter is arc-shaped or u-shaped, the adapter may couple to the device at one end of the arc-shaped housing, or at both ends of the arc-shaped housing, etc. Electrical contacts may be included at the interface locations (both on the adapter and on the hand-held device) to form an electrical path between the adapter and the hand-held device. The electrical path may be used to provide control signals from the hand-held device to the adapter. For example, the activation of RF energy may be initiated by an activation element on the hand-held device and control signal provided via the electrical path to activate RF energy generation and delivery. In instances where the RF probe further includes visualization sensors, switches and control elements on the hand-held device may still be used to provide and control the visualization capabilities and the RF energy capabilities.

### Additional Components and Functionality

In some aspects of the invention, the RF tissue modulation devices are configured to include additional components and the associated functionalities of the additional components. For example, the elongated member may further include components such as visualization sensors, lumens, illuminators, etc.

In some embodiments, the RF tissue modulation devices further include a visualization sensor integrated at the distal end of the elongated member, such that the visualization sensor is integrated with the elongated member. As the visualization sensor is integrated with the elongated member, it cannot be removed from the remainder of the elongated member without significantly compromising the structure and functionality of the elongated member. Accordingly, the devices of the present invention are distinguished from devices which include a “working channel” through which a separate autonomous device is passed through. In contrast to such devices, since the visualization sensor of the present device is integrated with the elongated member, it is not a separate device from the elongated member that is merely present in a working channel of the elongated member and which can be removed from the working channel of such an elongated member without structurally compromising the elongated member in any way. The visualization sensor may be integrated with the elongated member by a variety of different configurations. Integrated configurations include configurations where the visualization sensor is fixed relative to the distal end of the elongated member, as well as configurations where the visualization sensor is movable to some extent relative to the distal end of the elongated member. Movement of the visualization sensor may also be provided relative to the distal end of the elongated member, but then fixed with respect to another component present at the distal end, such as the plasma generator, a distal end integrated illuminator, etc. Specific configurations of interest are further described below in connection with the figures.

In some instances, the distal end integrated visualization sensor is present as an RF-shielded visualization module. As the visualization sensor module of these embodiments is RF-shielded, the visualization sensor module includes an RF shield that substantially inhibits, if not completely prevents, an ambient RF field from reaching and interacting with circuitry of the visualization sensor. As such, the RF shield is a structure which substantially inhibits, if not completely prevents, ambient RF energy (e.g., as provided by a distal end RF electrode, as described in greater detail blow) from impacting the circuitry function of the visualization sensor.

Visualization sensor modules of devices of the invention include at least a visualization sensor. In certain embodiments, the devices may further include a conductive member that conductively connects the visualization sensor with another location of the device, such as a proximal end location. Additional components may also be present in the visualization sensor module, where these components are described in greater detail below.

In some instances, the RF tissue modulation devices further include a second tissue modifier other than the plasma generator. Tissue modifiers are components that interact with tissue in some manner to modify the tissue in a desired way. The term modify is used broadly to refer to changing in some way, including cutting the tissue, ablating the tissue, delivering an agent(s) to the tissue, freezing the tissue, etc. As such, of interest as tissue modifiers are tissue cutters, tissue ablators, tissue freezing/heating elements, agent delivery devices, etc. Tissue cutters of interest include, but are not limited to: blades, liquid jet devices, lasers and the like. Tissue ablators of interest include, but are not limited to ablation devices, such as devices for delivery ultrasonic energy (e.g., as employed in ultrasonic ablation), devices for delivering plasma energy, devices for delivering radiofrequency (RF) energy, devices for delivering microwave energy, etc. Energy transfer devices of interest include, but are not limited to: devices for modulating the temperature of tissue, e.g., freezing or heating devices, etc. In some embodiments, the tissue modifier is not a tissue modifier that achieves tissue modification by clamping, clasping or grasping of tissue such as may be accomplished by devices that trap tissue between opposing surfaces (e.g., jaw-like devices). In these embodiments, the tissue modification device is not an element that is configured to apply mechanical force to tear tissue, e.g., by trapping tissue between opposing surfaces.

In some instances, as described elsewhere, the RF tissue modulation devices may include a collimated laser configured to emit collimated laser light from a distal region of the elongated member, such as the distal end of the elongated member. The collimated laser components of these embodiments may be configured for use for a variety of purposes, such as but not limited to: anatomical feature identification, anatomical feature assessment of sizes and distances within the field of view of the visualization sensor, etc.

In certain embodiments, devices of the invention include an image recognition module. Image recognition modules of interest are those that are configured to receive image data and compare the received image data with a reference that includes at least one of color descriptor data and anatomical descriptor data to make a determination as to whether an alert signal should be generated. In some embodiments, the devices may include a conveyance structure configured to convey an item between the distal end of the elongated member and an entry port positioned at a proximal end of the device, e.g., associated with the proximal end of the elongated member or associated with the hand-held control unit. This conveyance structure may have any convenient configuration, where in some instances it is a “working channel” disposed within the elongated member. When present as a working channel, the channel may have an outer diameter that varies, and in some instances has an outer diameter of 3 mm or less, such as 2 mm or less and including 1 mm or less. The conveyance structure may be configured to transport items, e.g., fluids, medicines, devices, to an internal target site or from an internal target site. As such, the proximal end entry port of the conveyance structure may vary, and may be configured to be operably coupled to a variety of different types of components, such as but not limited to: aspiration units, fluid reservoirs, device actuators, etc.

### Illustrated Embodiments

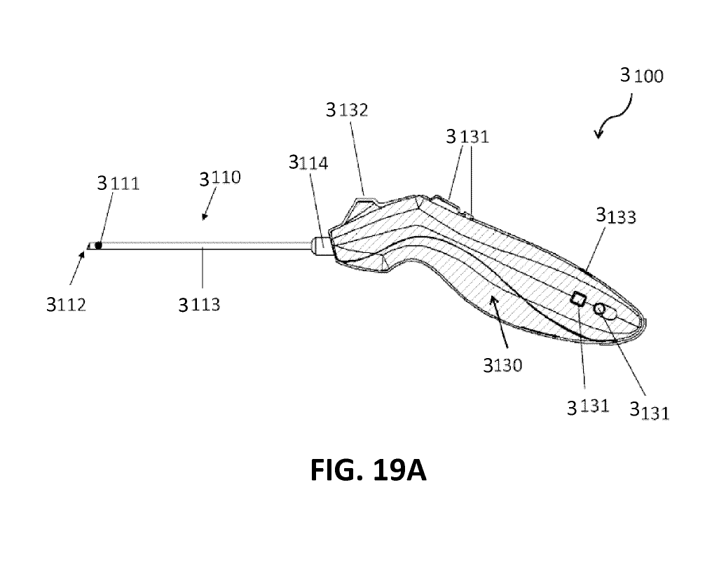

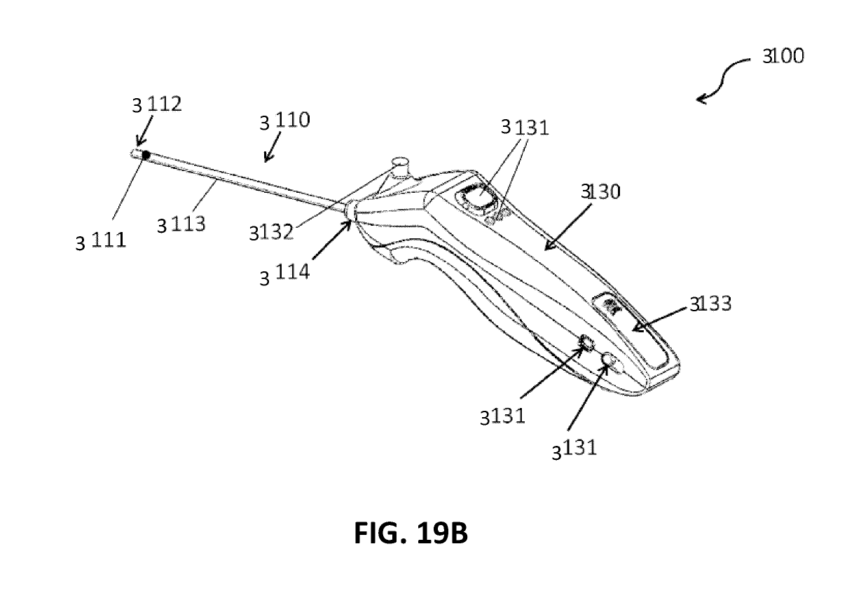

Turning now to the figures, FIGS. 19A-19B illustrate a side view and perspective view, respectively, of an RF tissue modification device comprising a hand-held control unit and RF probe, according to some embodiments. Both figures are described together in the following paragraphs.

The RF tissue modulation device 3100 is shown including a hand-held control unit 3130 and a removably coupled elongated member 3110 having a plasma generator 3111 at the distal end (also referred to herein as a “RF probe 3110”). From an external view, the RF probe 3110, as shown, includes a distal end tip 3112, and tubular structure 3113, and a mechanical connector 3114 to removably couple to the hand-held control unit 3130. The hand-held control unit 3130, from an external view may include various control switches 3131 for controlling the device—e.g., activating delivery of RF energy to the plasma generator 3111, turning power off and on, controlling the rotation or articulation of the RF probe 3110, controlling functions associated with illuminators, visualization, etc., if such capabilities are present, etc. It should be understood that the term switch is used generally and may include any various types of control elements, such as keys, buttons, wheels, etc. Furthermore, it should be understood that the control switches 3131 may be positioned in various locations on the hand-held control unit 3130.

While not required, positioning control switches 3131 in locations on the hand-held control unit 3130 that can be accessed by the user while gripping the control unit 3130 provides the advantage of being more user-friendly. This may be especially advantageous for control switches 3131 expected to be used more frequently. For example, one of the control switches 3131 may control the delivery of RF energy. Another one of the control switches 3131 may, for example, control motor rotation and three positions available for controlling the motor rotation, one position to rotate the motor clockwise, one position to rotate the motor counterclockwise, and a position in the center that is neutral.

Furthermore, as shown in FIG. 19A, there may be a battery door 3133 for the purpose of accessing the electrical energy source inside. As stated above, the electrical energy source may include one or more DC batteries, for example. The DC batteries may be rechargeable or non-rechargeable batteries. In some embodiments, the hand-held control unit may be configured to removably couple to a docking station, cradle, plug, etc. (not shown) to recharge the electrical energy source.

Internally, the hand-held control unit 3130 includes RF energy source components as described above. The hand-held control unit 3130 may include, for example, the electrical energy source, a voltage converter, charge accumulator, and RF signal generator (not shown). Example embodiments of the RF energy source are described in further detail in later figures illustrating example block diagrams of the RF energy source. It should be understood that additional circuitry such as wiring, LEDs, control units (e.g., microcontrollers and/or microprocessors), memory units (e.g., volatile and non-volatile memory) may also be included within the hand-held control unit.

An RF line (not shown) is positioned along the RF probe to electrically couple the hand-held control unit 3130 and the plasma generator 3111 positioned at the distal end of the RF probe 3110. The RF line may be, for example, conductive wiring extending within the RF probe 3110 from the mechanical connector 3114 to the RF electrode (not shown) of the plasma generator 3111. In some instances, RF probe 3110 includes RF shielding as described above.

In some instances, the RF probe 3110 may include additional components other than the plasma generator 3111 (e.g., visualization sensors, illumination elements, lumens, etc.). For example, in some embodiments, a visualization sensor may be included at a distal end of the RF probe 3110, and a monitor coupled to the hand-held control unit 3130 at an optional monitor connector 3132. In some embodiments, the hand-held control unit 3130 includes a built in monitor or display.

Hand-held minimally dimensioned diagnostic devices having integrated distal end visualization sensors and other additional components are discussed in U.S. application Ser. No. 12/501,336, the disclosure of which is hereby incorporated by reference. The components, their configurations, and operations thereof, described within the disclosure may also apply here to the RF probe 3110 and hand-held control unit 3130 of the RF tissue modulation devices 3100, when such components are present. For example, when visualization capabilities are included within device 3100, hand-held control unit 130 may include associated circuitry such as an image processor, video processor, and/or stereoscopic image module, as described in U.S. application Ser. No. 12/501,336. Additionally, tissue modification devices having tissue modifiers and other additional components are discussed in Provisional Application Ser. No. 61/082,774, U.S. application Ser. No. 12/422,176, and International Patent Application Serial No. US09/51446, the disclosures of which are herein incorporated by reference. The components, their configurations, and operations thereof, described within these disclosures may also apply here to the RF probe 3110 and hand-held control unit 3130 of the RF tissue modulation devices 3100, when such components are present.

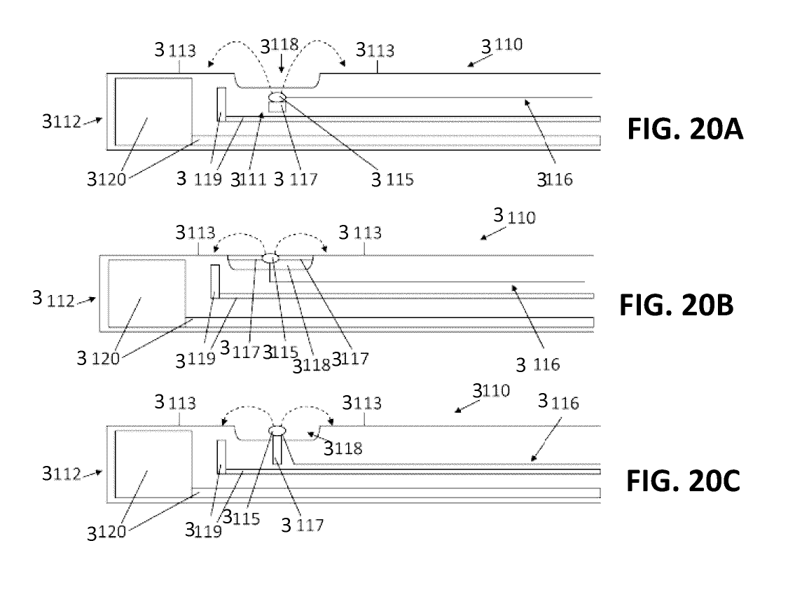

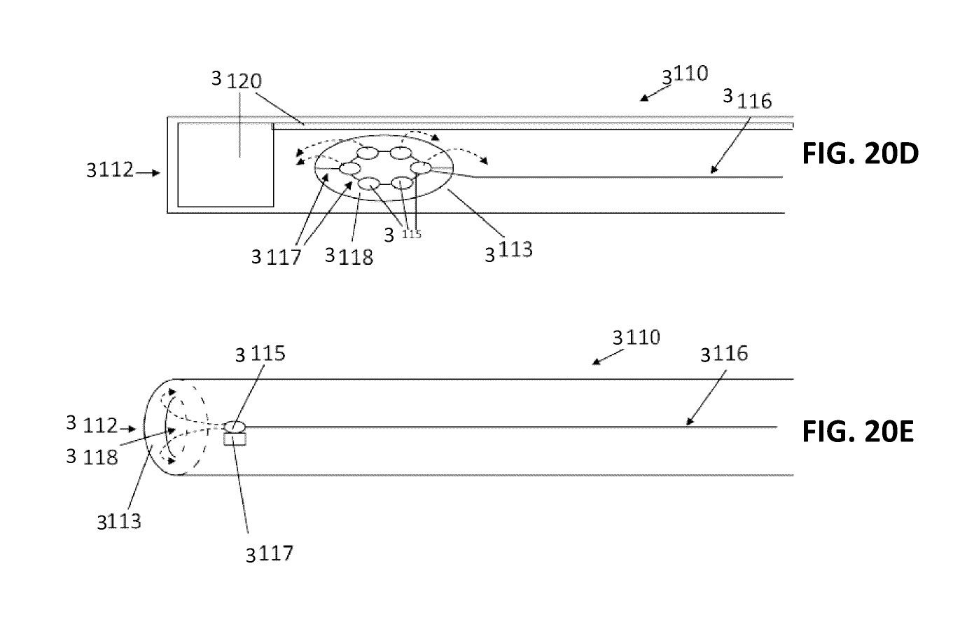

FIGS. 20A-20E illustrate a distal end of an elongated member 3110 including a plasma generator 3111, according to some embodiments. Plasma generator 3111 is shown to include a conductive member 3115 functioning as an RF electrode. The plasma generator may also include insulators and/or other conductive members such as other electrodes. Conductive member 3115 is maintained in position by insulator 3117. The conductive member 3115 is coupled to RF line 3116. RF line 3116 is shown extending from the conductive member 3115 at the distal end of the elongated member 3110 down the length of the elongated member to the proximal end of the elongated member 3110. RF line 3116 provides an electrical connection between the RF energy source (not shown) and the conductive member 3115 such that RF energy (e.g., high voltage modulated RF signals as described above) may be delivered to conductive member 3115 from RF energy source when RF energy is activated. When RF energy is activated and received by plasma generator 3111, plasma generator 3111 produces a plasma between the conductive member 3115 and outer surface 3113, for example, as represented by the dotted arrows illustrated in FIGS. 2A-2E.

For FIGS. 20A-20D, elongated member 3110 is shown to further include additional components 3120 (such as earlier described visualization sensors, illuminator elements, etc.) also at the distal end of the elongated member 3110. Additional components may also include components running the length of the elongated member 3110—e.g., wires, fiber optics, etc. It should be understood that the position of the additional components may vary depending on application, and are represented generally in FIGS. 20A-20D.

As further shown in FIGS. 20A-20D, elongated member may also include an RF shield 3119 within the elongated member 3110 and adjacent to the RF line 3116 and/or RF electrode 3115. RF shield 119 provides an ambient RF barrier between the additional components 3120 and RF line 116 and/or conductive member 3115.

FIG. 20A illustrates a cross sectional side view of an elongated member 3110, according to one embodiment. Elongated member 3110 includes an outer surface 3113, distal end opening 3118 within the outer surface 3113, and distal end tip 3112. In this embodiment, distal end opening 3118 is positioned over conductive member 3115. When RF energy is delivered to plasma generator 3111 via RF line 3116, a plasma is generated between the conductive member 3115 and outer surface of the elongated member 3113, as represented by the dotted arrows.

FIG. 20B illustrates a cross sectional side view of an elongated member 3110, according to one embodiment. Elongated member 3110 includes an outer surface 3113, distal end opening 3118 within the outer surface 3113, and distal end tip 3112. Conductive member 3115 is positioned within the distal end opening 3118 by insulator 3117. In this embodiment, insulator 117 is shown extending from elongated member 3110 near the perimeter of the opening 3118. When RF energy is delivered to plasma generator 3111 via RF line 3116, a plasma is generated between the conductive member 3115 and outer surface 3113, as represented by the dotted arrows.

FIG. 20C illustrates a cross sectional side view of an elongated member 3110, according to one embodiment. Elongated member 3110 includes an outer surface 3113, distal end opening 3118 within the outer surface 3113, and distal end tip 3112. Conductive member 3115 is positioned within the distal end opening 3118 by insulator 3117. In this embodiment, insulator 117 extends from within the elongated member 3110. When RF energy is delivered to plasma generator 3111 via RF line 3116, a plasma is generated between the conductive member 3115 and outer surface 3113, as represented by the dotted arrows.

FIG. 20D illustrates a cross sectional top view of an elongated member 3110, according to one embodiment. Elongated member 3110 includes an outer surface 3113, distal end opening 3118 within the outer surface 3113, and distal end tip 3112. Conductive member 3115 is positioned within the distal end opening 3118 by insulator 3117. In this embodiment, insulator 3117 is shown extending from elongated member 3110 near the perimeter of the opening 3118. Furthermore, in this embodiment, multiple insulators 3117 and conductive members 3115 are shown. As shown, insulators 3117 may be positioned between conductive members 3115 to maintain the conductive members 3115 in position. It should be understood that RF line may extend within the insulator 3117 in some instances—e.g., metal wiring extending through a piece of ceramic and contacting the RF electrode. When RF energy is delivered to plasma generator 3111 via RF line 3116, a plasma is generated between the conductive members 3115 and outer surface 3113, as represented by the dotted arrows.

FIG. 20E illustrates a cross sectional side view of an elongated member 3110, according to one embodiment. Elongated member 3110 includes an outer surface 3113, distal end opening 3118 within the outer surface 3113, and distal end tip 3112. In this embodiment, distal end opening 3118 is positioned over conductive member 3115 at the distal end tip 120 of the elongated member 3110. When RF energy is delivered to plasma generator 3111 via RF line 3116, a plasma is generated between the conductive member 3115 and outer surface 3113, as represented by the dotted arrows. It should be understood, that while the FIG. 20E is shown not to include additional components 3120, this embodiment is exemplary and additional components 3120 may be included in other embodiments having the distal end opening 3118 at the distal end tip 3112. Moreover, it should be understood that elongated members shown in FIGS. 20A-20D, may not include additional components 3120 and/or RF shielding 3119 in other embodiments. It should also be understood that the embodiments shown for FIGS. 2A-2E are illustrative and are functionally represented to facilitate understanding of the configurations and placements of the components. For example, the embodiments shown are not drawn to scale and do not embody the exact shapes of the components used.

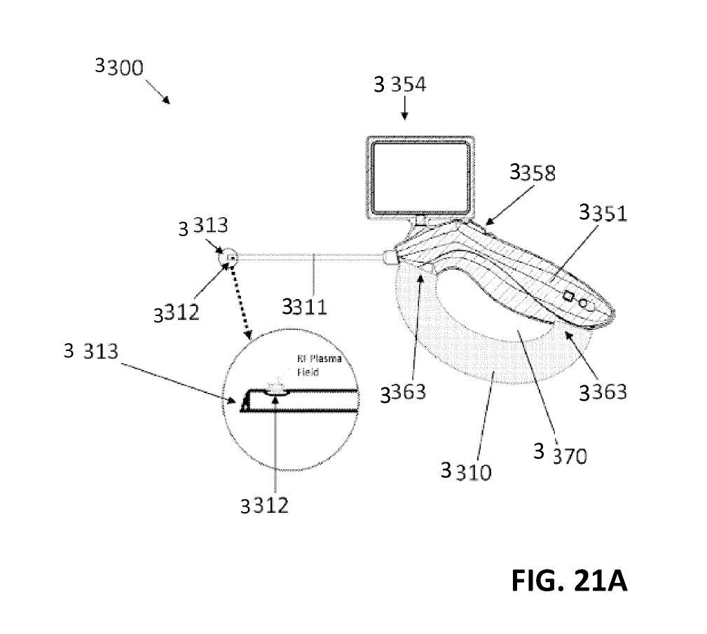

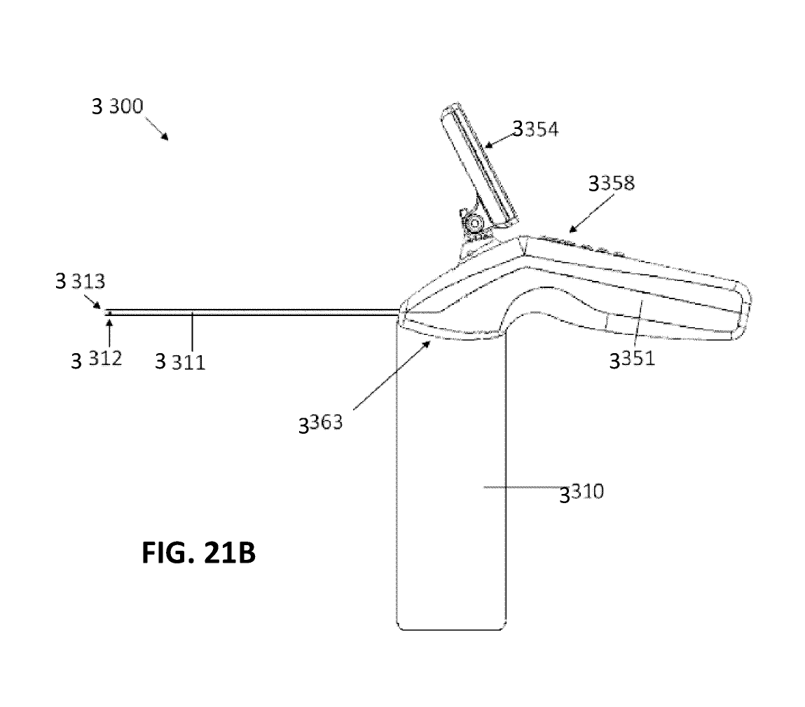

FIG. 21A-21B illustrate RF tissue modulation devices including an adapter, elongated member, and hand-held piece, according to some embodiments. It should be understood that in some instances the hand-held piece and elongated member may function without the adapter as a diagnostic device, such as a visualization device. For example, the visualization device may be similar to the visualization devices described in U.S. application Ser. No. 12/501,336, except configured to removably couple to the adapter.

As shown in FIG. 21A, RF tissue modulation device 300 includes a hand-held piece 3351 and an elongated member 3311 (e.g., a RF probe) coupled to the hand-held piece 3351. Hand-held piece 3351 is shown having a monitor 3354 and control switches 3358 coupled to hand-held piece 3351. In this embodiment, elongated member 3311 is removably coupled to the hand-held piece 3351 at a distal end of the hand-held piece 3351. The elongated member 352 includes a RF generator 3312 and visualization sensor 3313 at a distal end of the elongated member 3311 used to provide visualization to monitor 3354 coupled to the hand-held piece 3351. The distal end of the elongated member 3311 is shown close up in FIG. 21, as represented by the dotted arrow and circled sections. Furthermore, as explained earlier, additional components as well as the visualization sensor may be included in the RF probe 3311—e.g., illuminators, lumens, etc.

While in this example embodiment, a visualization sensor 3313 is included in the elongated member 3311, it should be understood that a visualization sensor may not be included in another embodiment. Additionally, it should be understood that the elongated member 3311 may be removed from hand-held piece 3351 and a new elongated member may be coupled in its place. For example, in some instances, an RF probe without visualization capabilities may be coupled to the hand-held piece 3351 instead. Furthermore, it should be understood that, in some instances, an elongated member (e.g., a visualization probe) without a RF plasma generator may be used in place of the RF probe, in which case the hand-held piece 3351 and visualization probe function as a visualization device (with or without the adapter 3310 coupled).

Adapter 3310 is shown having an arc-shape or u-shape and removably coupled to hand-held piece 3351. Adapter 3310 is form fitted to couple to the hand-held piece 3351 at interface locations 3363 and to provide a space 3370 between the inner arc or “u” of the adapter 3310 and the hand-held piece 3351, thus allowing the user to grip the hand-held piece 3351 without the adapter 3310 obstructing the user's grip.

Internally, adapter 3310 includes RF energy source components (not shown). For example, in some embodiments, the adapter 3310 includes an electrical energy source, a charge accumulator, voltage converter, and RF signal generator, wherein the voltage converter, charge accumulator, and RF signal generator operably couple the electrical energy source to the plasma generator 3312 on the elongated member 3311 removably and operably coupled to the hand-held piece 3351. Example embodiments of the RF energy source are described in further detail in later figures illustrating example block diagrams of the RF energy source. It should be understood that additional circuitry such as wiring, LEDs, control units (e.g., microcontrollers and/or microprocessors), memory units (e.g., volatile and non-volatile memory) may also be included within the adapter. Furthermore, in some instances, the adapter 310 may additionally include a bandpass filter and/or RF tuner.

An RF line (not shown) is positioned within RF probe 3311 to electrically couple the adapter 310 and the plasma generator 3312 positioned at the distal end of the RF probe 3311. The RF line may be, for example, conductive wiring extending within the RF probe 3311 from an RF electrode (not shown) of the plasma generator 3312. In some instances, RF probe 3311 includes RF shielding as described above.

Adapter 3310 is configured to couple to the hand-held piece 3351 at interface locations 3363. Electrical contacts (not shown) may be provided at interface locations 3363 on both the adapter 3310 and the hand-held piece 3351 to provide an electrical path between the two. The electrical path provides an electrical path for the delivery of RF energy from the adapter to the plasma generator. Furthermore, the electrical path provides a communication path between the adapter 3310 and the hand-held piece 3351

As stated above, the RF probe 3311 coupled to the adapter 3310 includes a visualization sensor 3313 in addition to a plasma generator 3312. In such case, the hand-held piece 3351 and adapter 3310 are configured such that the hand-held piece 3351 may operate with the visualization sensors 3313 and plasma generator 3312 on the RF probe 3311. Further, the hand-held piece 3351 includes various switches 3358 to control functions of the hand-held piece 3351 and adapter 3310—e.g., switches to activate the delivery of RF energy to the plasma generator, switches for controlling visualization, lighting, rotation, articulation, etc. When RF energy is activated (e.g., by the user depressing a corresponding control switch 3358, the RF energy source within adapter generates RF energy (e.g., the high voltage modulated RF signal described earlier) and delivers it to the plasma generator 3312 via the RF line.

Adapter 3310 may further include a battery door (not shown) for removing the electrical energy source—e.g., chargeable or non-chargeable DC batteries. In some instances, the rechargeable batteries cannot be removed by the user and the adapter configured to removably couple to a docking station, cradle, plug, etc. In such case, the adapter may include a corresponding charging plug, port, etc. In some instances, the adapter is configured to be charged via electrical contacts at the interface locations 3363.

While this embodiment is described as having two interface locations, it should be understood that in other embodiments, the RF tissue modulation device may include another number of interface locations—e.g., one. Furthermore, it should be understood that when there are more than one interface location, electrical contacts may be included at one or more of the interface locations. Moreover, the electrical path for delivery of RF energy is not required to be at the same interface location of the electrical path for communication between the hand-held piece and the adapter.

The description above for FIG. 21A applies to FIG. 21B as well, except in FIG. 3B the adapter 3310 is generally shaped as a rectangle as opposed to an are or u-shape, and is configured to couple to the hand-held piece 3351. The rectangular shaped adapter 310 is configured to removably and operably couple to interface location 3363 of hand-held piece 3351 of RF tissue modulation device 3300. Interface location 3363 may, for example, include a socket, plug, or other coupling mechanism for coupling the adapter 3310 to the hand-held piece 3351. Upon coupling, contacts (not shown) from the adapter 3310 at interface location 3363 and contacts (not shown) from the hand-held piece form an electrical path for delivery of control signals, as well as delivery of RF energy from the adapter 3310 to the plasma generator 3312, as similarly described above. It should be understood that a variety of shapes and interface locations may be implemented without compromising the underlying principles of the invention.

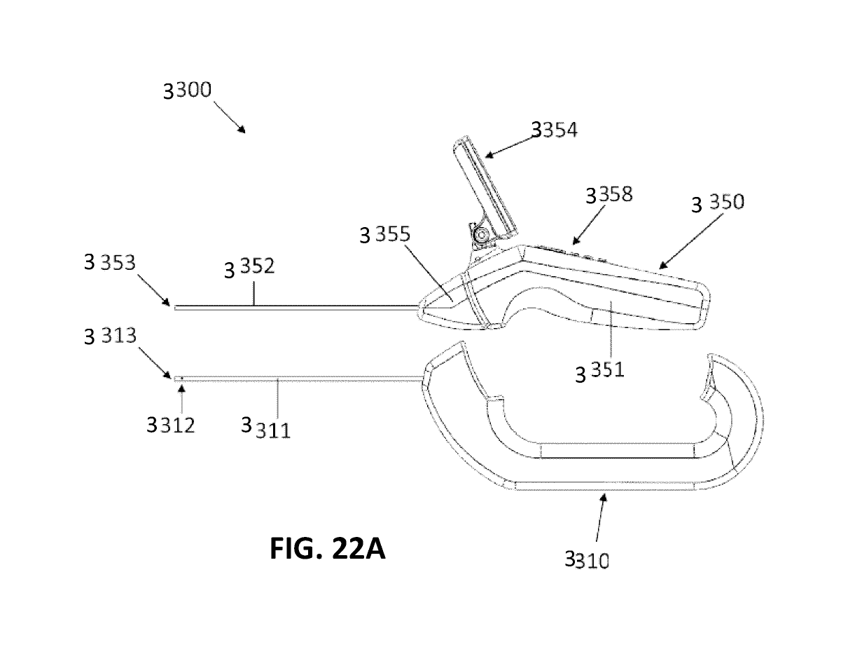

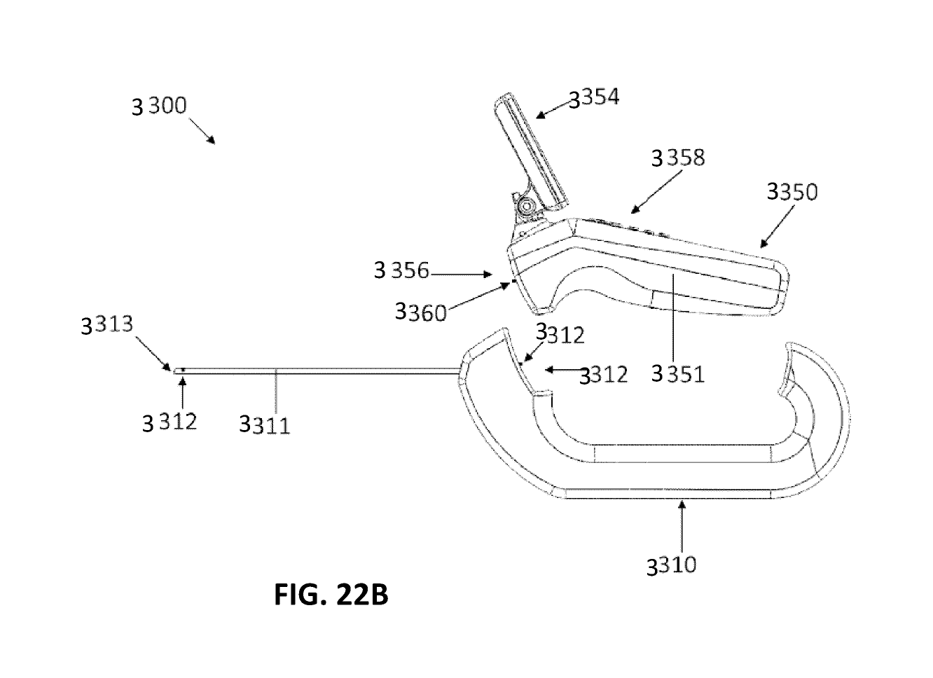

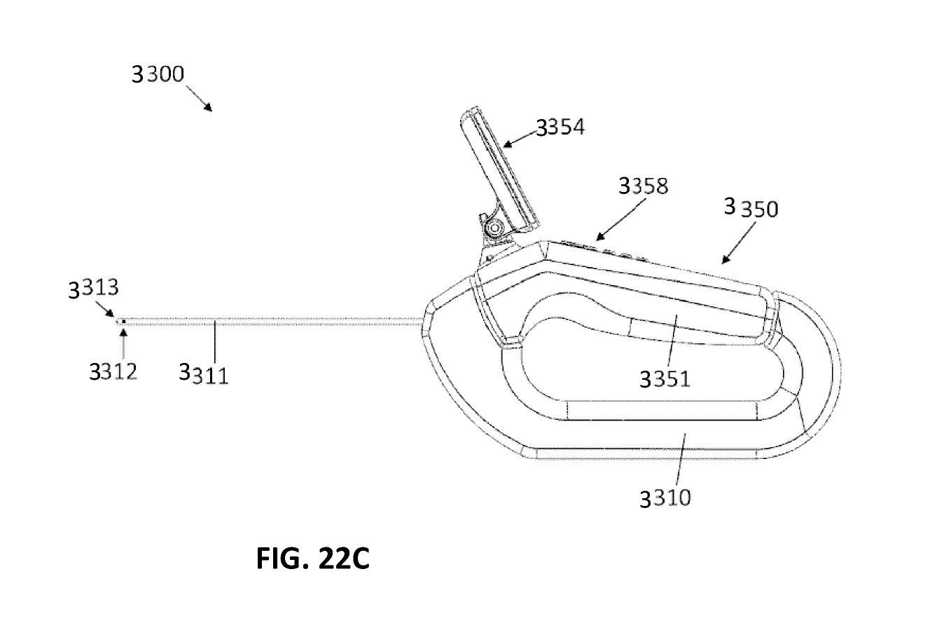

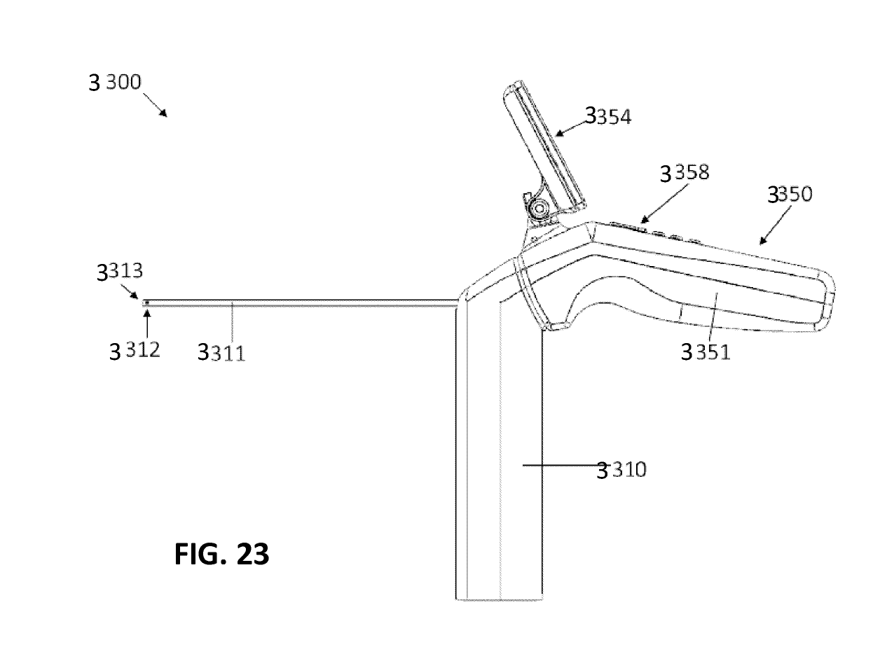

FIGS. 22A-22C and FIG. 23 illustrate an RF tissue modulation device 300 including an adapter 3310 and diagnostic device 3350 (also referred to herein as “visualization device”), according to one embodiment. The visualization device may be similar to the visualization devices described in U.S. application Ser. No. 12/501,336, except configured to removably couple to the adapter. More specifically, FIGS. 22A-22C and FIG. 23 illustrate various embodiments where the elongated member (e.g., RF probe) is removably coupled to the adapter.

As shown in FIG. 22A, visualization device 3350 includes a hand-held piece 3351 and an elongated member 3352 (e.g., a visualization probe) coupled to the hand-held piece 351. Hand-held piece is shown having a monitor 3354 and control switches 3358 coupled to hand-held piece 3351. The elongated member 3352 includes a visualization sensor 3353 at a distal end of the elongated member 3352 used to provide visualization to monitor 354 coupled to the hand-held piece 3351. In this embodiment, elongated member 3352 is removably coupled to the hand-held piece 3351 at a removable section 3355 of the hand-held piece 351. Removable section 3355 is removed when adapter 3310 is to be operably coupled to the hand-held piece 3351.

Adapter 3310 is shown having an arc-shape or u-shape with RF probe 3311 removably coupled to adapter 3310. The RF probe includes a plasma generator 3312 and visualization sensor 3313 at a distal end. Adapter 3310 is form fitted to couple to the visualization device 3350 at interface location 3356 and to provide a space between the inner are or “u” of the adapter 3310 and the hand-held device 3350, thus allowing the user to grip the hand-held device 3350 without the adapter 3310 obstructing the user's grip.

Internally, adapter includes RF energy source components (not shown). For example, in some embodiments, the adapter includes an electrical energy source, a charge accumulator, voltage converter, and RF signal generator, wherein the voltage converter, charge accumulator, and RF signal generator operably couple the electrical energy source to the plasma generator 3312 on an elongated member 3311 (e.g., RF probe) removably and operably coupled to the adapter 3310. Example embodiments of the RF energy source are described in further detail in later figures illustrating example block diagrams of the RF energy source. It should be understood that additional circuitry such as wiring, LEDs, control units (e.g., microcontrollers and/or microprocessors), memory units (e.g., volatile and non-volatile memory) may also be included within the adapter. Furthermore, in some instances, the adapter 3310 may additionally include a bandpass filter and/or RF tuner.

Elongated member 3311 is shown removably coupled to adapter 3310 and includes a plasma generator 3312 and visualization sensor 3313 at a distal end of the elongated member 3311. While in this example embodiment, a visualization sensor is included in the elongated member, it should be understood that at visualization sensor may not be included in another embodiment. Furthermore, as explained earlier, additional components as well as the visualization sensor may be included in the RF probe 3311—e.g., illuminators, lumens, etc.

An RF line (not shown) is positioned within RF probe 3311 to electrically couple the adapter 310 and the plasma generator 3312 positioned at the distal end of the RF probe 3311. The RF line may be, for example, conductive wiring extending within the RF probe 3311 from an RF electrode (not shown) of the plasma generator 3312. In some instances, RF probe 3311 includes RF shielding as described above.

To couple the adapter 3310 to the visualization device 3350, the removable section 3355 of the hand-held piece 3351, along with elongated member 3352, are removed, as illustrated in FIG. 22B. Removable section 3355 is removably coupled to hand-held piece 3351 at an interface location 3356. Interface location 3356 may include electrical contacts 3360 that contact contacts on the removable section 3355, thus forming an electrical path between the hand-held piece 3351 and visualization probe 3352.

Adapter 3310 is configured to couple to the hand-held piece 3351 at an interface location 3356 where the removable section 3355 was coupled, as illustrated in FIG. 22C. In this way, the electrical contacts 3360 at interface location 3357 on the hand-held piece 3351 that were providing an electrical path between the hand-held piece 3351 and the elongated member 3352 are now used to provide an electrical path between the hand-held piece 3351 and contacts 3361 on the interface location 3357 of adapter 3310 when coupled.

As stated above, the RF probe 3311 coupled to the adapter 310 includes a visualization sensor 3313 in addition to a plasma generator 3312. In such case, the hand-held device 3350 and adapter 3310 are configured such that the hand-held device 3350 may operate with the visualization sensors 3313 and plasma generator 3312 on the RF probe 3311. Further, the visualization device 3350 includes various switches 3358 to control functions of the device 3350 and adapter 3310—e.g., switches to activate the delivery of RF energy to the plasma generator, switched for controlling visualization, lighting, rotation, articulation, etc. When RF energy is activated (e.g., by the user depressing a corresponding control switch 358, the RF energy source within adapter generates RF energy (e.g., the high voltage modulated RF signal described earlier) and delivers it to the plasma generator 3312 via the RF line.

While in this example embodiment, removable section is removed in order to operably couple the hand-held piece, in another embodiment, the adapter operably couples to the hand-held piece without requiring a removable section to be included on the hand-held piece. In such case, the RF probe 3311 removably couples to the hand-held piece at the same location that the visualization probe 3352 is removably coupled.

Adapter 3310 may further include a battery door (not shown) for removing the electrical energy source—e.g., chargeable or non-chargeable DC batteries. In some instances, the rechargeable batteries cannot be removed by the user and the adapter configured to removably couple to a docking station, cradle, plug, etc. In such case, the adapter may include a corresponding charging plug, port, etc. In some instances, the adapter is configured to be charged via electrical contacts 361.

FIG. 23 illustrates an RF tissue modulation device 3300 including an adapter 3310 and diagnostic device 350 (also referred to herein as “visualization device”), according to one embodiment. The description above for FIGS. 22A-22C apply to FIG. 23 as well, except in FIG. 5 the adapter 3310 is generally shaped as a rectangle as opposed to an are or u-shape. The rectangular shaped adapter 3310 is configured to removably and operably couple to the interface location 3356 of hand-held piece 3351 of visualization device 3350.

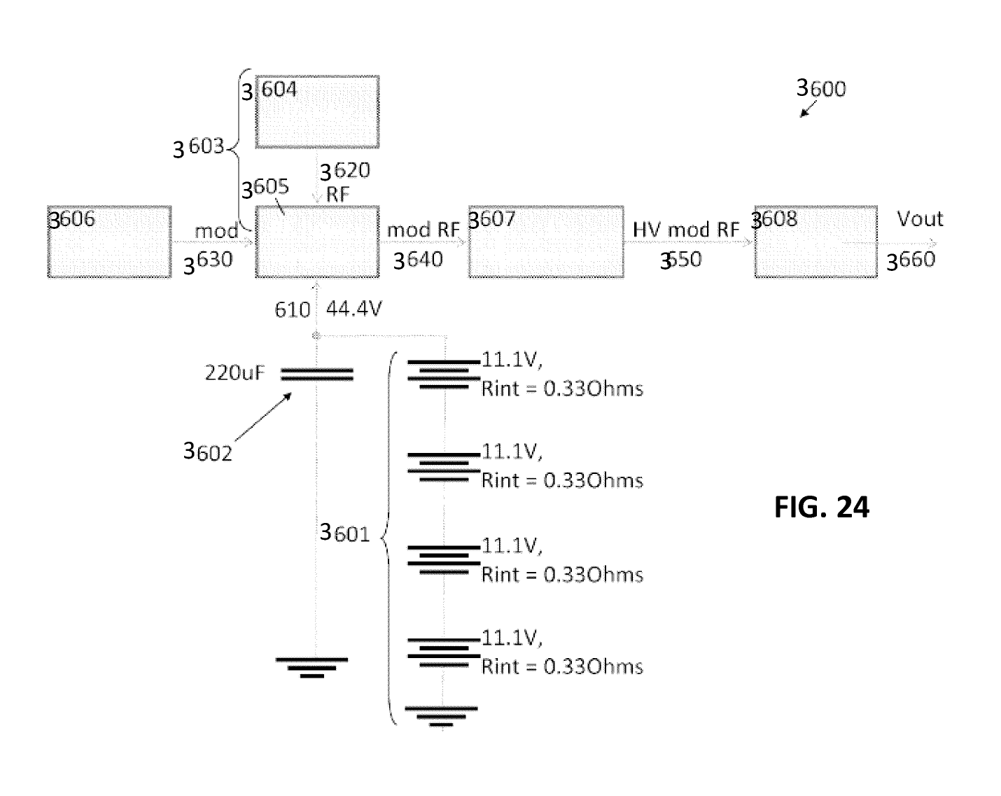

Turning now to the RF energy source, FIG. 24 illustrates a functional block diagram of an RF energy source, according to one embodiment. As shown, RF energy source 3600 includes an electrical energy source 3601 coupled to a charge accumulator 3602. Electrical energy source 3601 provides electrical energy for storage in charge accumulator 3602. Electrical energy source 3601 may comprise one or more DC power sources (e.g., batteries) to provide the electrical energy for storage in charge accumulator 3602, shown here as a capacitor. For example, in the embodiment shown, electrical energy source 3601 comprises four 11.1 volt batteries connected in series and provides a combined DC voltage of 44.4 volts across charge accumulator 3602. Charge accumulator 3602 and electrical energy source 3601 are shown coupled to RF signal generator 3603.

In the embodiment shown, DC voltage from electrical energy source 3601 is provided across charge accumulator 3602 and charge is stored therein. In some instances, charging may occur when delivery of RF energy to the plasma generator is not activated by the user. When RF energy is activated, charging of the charge accumulator 3602 is interrupted and the stored energy in the charge accumulator 3602 is discharged. For example, electrical energy source 3601 may be disconnected from charge accumulator 3602 by a switch (not shown) triggered by a control signal received from the hand-held control unit or medical device upon depression of the control switch for activation of RF energy. For example, charge accumulator 3602 may be decoupled from electrical energy source 3601 and begin discharging. The discharging of the charge accumulator 3602 provides a voltage signal 3610 to the RF signal generator 3603.

The RF tissue modulation devices may be configured to deliver RF energy from the RF energy source to the plasma generator for a therapeutic duration. The therapeutic duration may range, for example, from minutes or less, such as 1 minute or less, including 30 seconds or less, such as 10 seconds or less. In some instances, the therapeutic duration may range from 1 to 2 seconds. The therapeutic duration may be controlled using a variety of implementations. For example, in some instances, a timer (not shown) may be used to return switches back to positions for charging—e.g., switches that couple/uncouple the charge accumulator to the electrical energy source. As another example, in some instances, recharging of the charge accumulator may not occur until the user releases the activation switch—e.g., thus coupling the charge accumulator back to the electrical energy source.

After delivery of RF energy to the plasma generator, the electrical energy source 3601 is again coupled to the charge accumulator 3602 and charging may occur again. In some instances, the RF tissue modulation device is configured to recharge the charge accumulator within a minimum recharge period between plasma generation. The minimum recharge period may range, for example, from 10 minutes or less, including 5 minutes or less, such as 3 minutes or less. In some instances, the minimum recharge period ranges from 1 to 2 minutes. Various recharge periods can be implemented by varying, for example, battery and capacitance sizes.

RF signal generator 3603 is shown comprising an RF power amplifier 3605 and RF clock source 3604. RF power amplifier 3605 is coupled to RF clock source 3604 and receives an RF clock signal 3620 as its input. RF power amplifier 3605 receives the RF clock signal 3620, as well as a bias voltage 3610 from charge accumulator 3602, and generates an amplified RF signal with an operating frequency based on the RF clock signal 3620 and peak voltage based on the bias voltage 3610 (e.g., in this case approximately 44 volts).

RF signal generator 3603 is shown also coupled to a second clock source 3606 providing a second clock signal 3630 for generating a modulated output signal based on the second clock signal 3630. Again, the modulation waveform may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 50 Hz. The RF signal is modulated at the modulation frequency based on a second clock and a modulated RF signal is output from the RF signal generator 3603. Thus, in such case, the RF signal generator 603 generates a modulated RF signal 3640 and outputs it to voltage converter 607.

RF signal generator 3603 is coupled to a voltage converter 3607, such as the transformer shown. Voltage converter 3607 steps up the voltage level of the modulated RF signal 640 received and outputs a high-voltage modulated RF signal 3650. While it should be understood that a voltage boost is not necessarily required if the electrical energy source 3601 provides sufficient voltage to begin with; however, in typical applications, practical design considerations (e.g., weight and size) limit the batteries to a voltage level which requires further boosting. In the embodiment shown, voltage converter 3607 is a 1:11 transformer which boosts the voltage level of the modulated RF signal 3640 to a high-voltage modulated RF signal 3650 with approximately 11 times the voltage amplitude. For example, if the modulated RF signal 3640 is at approximately 44 volts, then the high-voltage modulated RF signal 3650 would have a voltage of approximately 484 volts.

Also shown in this embodiment is an optional RF tuner 3608 coupled to voltage converter 3607. RF tuner 3608 receives the high voltage modulated RF signal 3650 and outputs a signal 3660 to the plasma generator—e.g., vian RF line. Signal 3660 is a high voltage modulated RF signal that has been tuned as follows. The RF tuner 3608 includes basic electrical elements (e.g., capacitors and inductors) which serve to tailor the output impedance of the RF energy system. The term “tailor” is intended here to have a broad interpretation, including affecting an electrical response that achieves maximum power delivery, affecting an electrical response that achieves constant power (or voltage) level under different loading conditions, affecting an electrical response that achieves different power (or voltage) levels under different loading conditions, etc. Furthermore, the elements of the RF tuner 3608 can be chosen so that the output impedance is dynamically tailored, meaning the RF tuner 3608 self-adjusts according to the load impedance encountered at the electrode tip. For instance, the elements may be selected so that the electrode has adequate voltage to develop a plasma corona when the electrode is placed in a saline solution (with saline solution grounded to return electrode), but then may self-adjust the voltage level to a lower threshold when the electrode contacts tissue (with tissue also grounded to return electrode, for example through the saline solution), thus dynamically maintaining the plasma corona at the electrode tip while minimizing the power delivered to the tissue and the thermal impact to surrounding tissue. RF tuner 3608, when present, can provide a number of advantages. For example, delivering RF energy to target tissue through the distal tip of the electrode is challenging since RF energy experiences attenuation and reflection along the length of the conductive path from the RF energy system to the electrode tip, which can result in insertion loss. Inclusion of an RF tuner 3608, e.g., as described above, can help to minimize and control insertion loss.

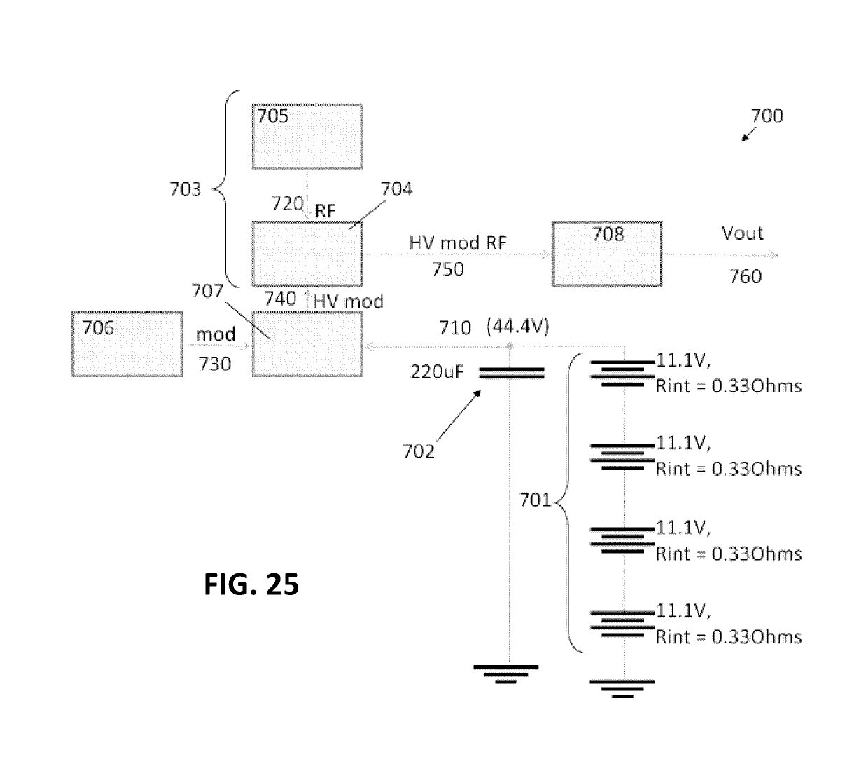

FIG. 25 illustrates a functional block diagram of an RF energy source, according to one embodiment. As shown, RF energy source 700 includes an electrical energy source 701 coupled to a charge accumulator 702. Again, electrical energy source 701 is shown as a series of 11.1 volt DC batteries to provide a voltage of approximately 44.4V to the charge accumulator 702 shown in this case to be a capacitor. Electrical energy provides the electrical energy for storage in charge accumulator 702 that discharged when activation of RF energy occurs. The above description for the charge accumulator and electrical energy source of FIG. 6 apply here as well, except the discharge of the capacitor is received by a voltage converter.

In this embodiment, voltage converter 707, shown here as a DC to DC converter, is coupled to charge accumulator 702 and receives the discharged voltage signal 710 from the charge accumulator 702. Voltage converter 707 boosts the voltage signal 710 received by the charge accumulator 702 to generate a high voltage output signal. Voltage converter 707 is also shown coupled to a clock source 706. The voltage converter 707 is configured to receive a clock signal 730 from the clock source 706 for modulation purposes and to output the high voltage signal at a modulated rate.

In some instances, the modulation at the modulation frequency comprises attenuating the amplitude of the high voltage signal based on the second clock signal. The modulation waveform (i.e., the clock signal from the clock source) may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 50 Hz.

For example, the clock signal 730 may be coupled to the enable input of voltage converter 707. In this way, the voltage converter 707 boosts the voltage signal 710 when enabled (e.g., when the clock signal 730 is high) and does not output a signal when disabled (e.g., when the clock signal 730 is low). Thus, the high voltage signal is modulated at a modulation frequency based on the clock signal from the clock source to generate a high voltage modulated signal 740.

Voltage converter 707 is also shown coupled to RF signal generator 3703. RF signal generator 703 receives the high voltage modulated signal 740 from voltage converter 707 and outputs a high voltage modulated RF signal 750. RF signal generator 3703 is shown comprising an RF power amplifier 704 and RF clock source 705. RF power amplifier 3704 is coupled to RF clock source 705 and receives an RF clock signal 720 from the RF clock source 705. Further, RF power amplifier 704 receives the high voltage modulated signal 740 from voltage converter 707 as a bias voltage.

The RF power amplifier 704 generates an amplified RF signal with an operating frequency based on the RF clock signal 720 and peak voltage based on the bias voltage (i.e., the high voltage modulated signal 740 from voltage converter 707). The resulting high voltage modulated RF signal 750 is output by the RF signal generator 703. In some instances, as shown, the RF signal generator 703 outputs high voltage modulated RF signal 750 to an optional RF tuner 708. RF tuner 708 receives the high voltage modulated RF signal and generates a tuned high voltage modulated RF signal 760, as similarly described above for FIG. 24.

FIG. 8 illustrates a high level functional block diagram of an RF energy source 800, according to one embodiment. As shown, RF energy source 800 includes an electrical energy source 801 coupled to a voltage converter 802. The electrical energy source 801 may comprise one or more DC power sources (e.g., batteries) to provide voltage 811 to a voltage converter 802. The voltage converter 802 boosts the voltage 811 provided by the electrical energy source 801 to provide a high voltage signal 812. The voltage converter 802 is coupled to a charge accumulator 803 and the high voltage output 812 from the voltage converter 802 provides electrical energy for storage within charge accumulator 803.

Charge accumulator 803 stores the electrical energy until RF energy is activated, at which point the electrical energy is discharged from the charge accumulator 803 as a high voltage modulated output signal 813. Charge accumulator is coupled to RF signal generator 804 and high voltage modulated output signal 813 is received by RF signal generator 804. In one embodiment, charge accumulator 803 discharges the stored energy in stages. In some instances, a modulation circuit is implemented to discharge stages of energy at a specific frequency and duty cycle, thus providing the modulated aspect of the high voltage modulated output signal 813.

The RF signal generator 804 receives the high voltage modulated signal 813 from charge accumulator 803 and outputs a high voltage RF signal 814 at a specific operating frequency. The RF signal generator 804 outputs the high voltage modulated RF signal 814 to an optional RF tuner 805. RF tuner 805 receives the high voltage modulated RF signal 814 and provides a tuned high voltage modulated RF signal as similarly described above for FIG. 24.

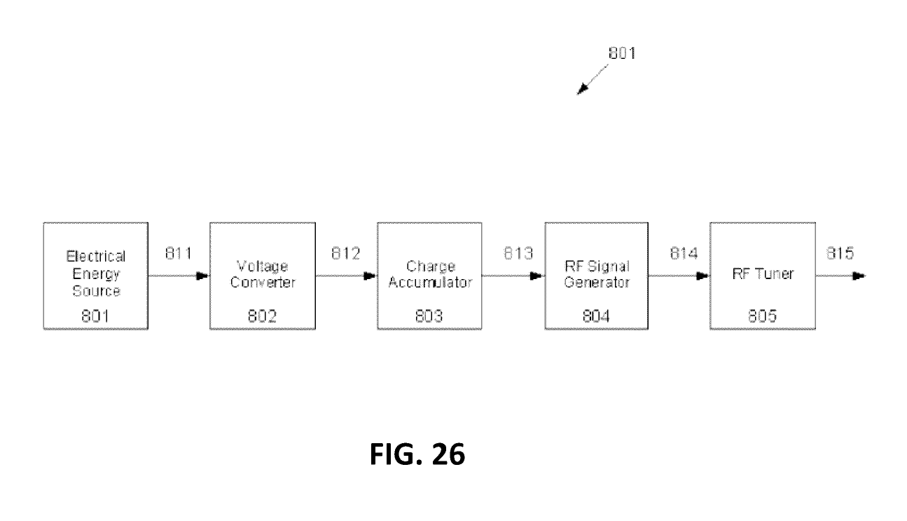

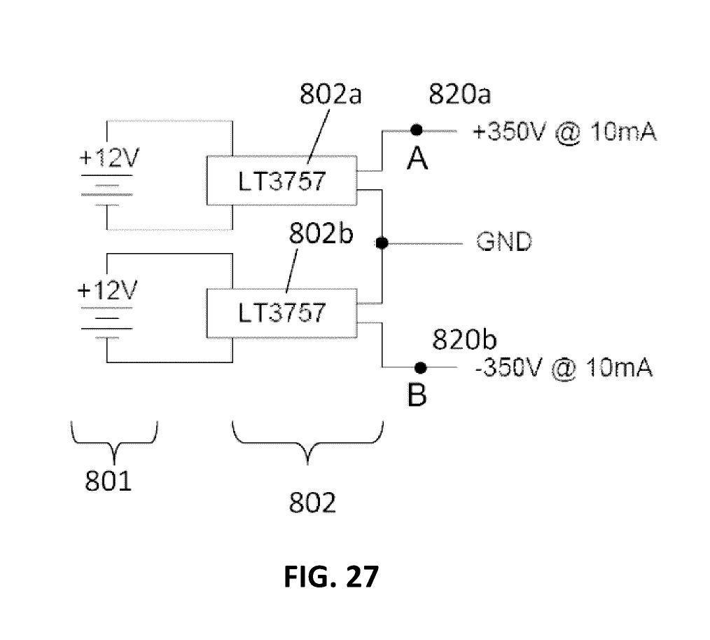

FIGS. 27-30 illustrate functional block diagrams corresponding to various elemental blocks shown in FIG. 26, according to certain embodiments. FIG. 27 illustrates a functional block diagram of the electrical energy source 801 and voltage converter 802 as shown in FIG. 8, according to one embodiment. Voltage converter 802 is shown to generally include voltage converter 802a and voltage converter 802b. In the embodiment shown, electrical energy source 801 comprises two DC power sources (e.g., batteries) with each coupled to separate voltage converters 802a,802b. The two voltage converters 802a,802b are configured to provide positive and negative high voltage rails at Point A and Point B shown in FIG. 27, respectively, with a common ground 821. A variety of devices may be used to perform such voltage conversion—e.g., two LT3757 DC-DC controllers by Linear Technologies as shown. In this embodiment shown, voltage converter 802a is configured to step up the voltage of a 12 volt battery to generate a positive high voltage output signal 820a at a positive rail. The second voltage converter 802 is configured to step up the voltage of a second 12 volt battery to generate a negative high voltage output signal 820b at a negative rail. Ranges of positive and negative high voltage outputs 820a,820b may vary depending on the particular application and design considerations. For instance, in some cases, positive and negative high voltage outputs 820a,820b may range from +/−50 volts (at e.g., approximately 1.4 mA) to +/−1000 volts (at e.g., approximately 28.5 mA), such as from +/−200 volts (at e.g., approximately 5.7 mA) to +/−500 volts (at e.g., approximately 14.2 mA), and including from +/−300 volts (at e.g., approximately 8.5 mA) to +/−400 volts (at e.g., approximately 11.4 mA). In some embodiments, the positive and negative high voltage outputs are +/−350 volts (at e.g., approximately 10 mA), as shown. Voltage levels may depend on the particular application and design considerations (e.g., voltage and current limits, etc.).

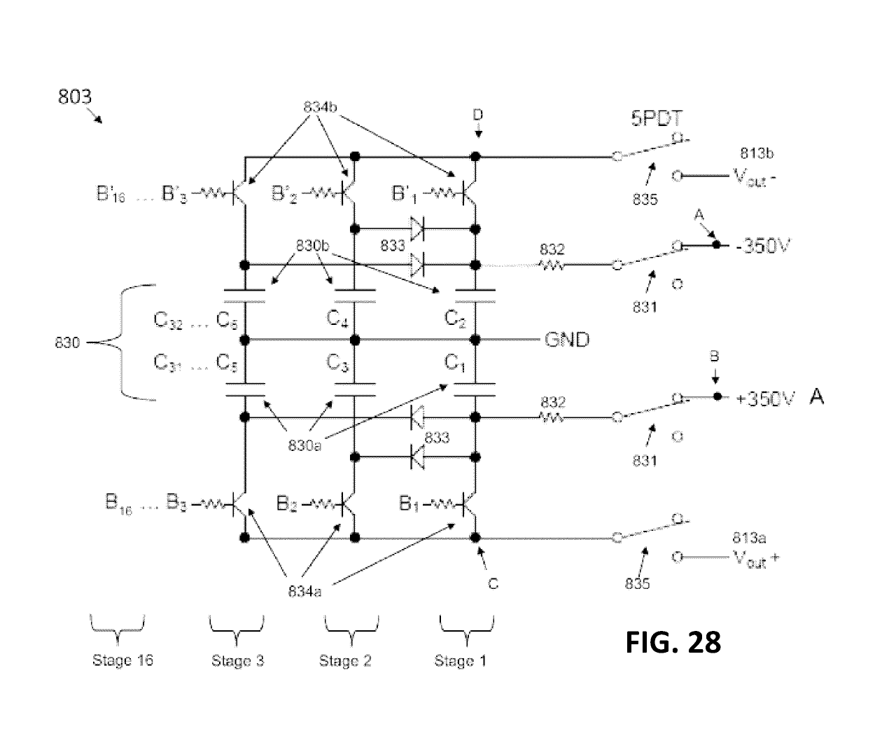

FIG. 28 illustrates a functional block diagram of the charge accumulator 803 shown in FIG. 26, according to one embodiment. Charge accumulator 803 may comprise one or more capacitors 830 configured to store electrical energy from the positive and negative high voltage outputs 820a,820b received by voltage converter 802 at corresponding Point A and Point B shown in FIG. 28. Switches 831 are shown in positions to allow the signals 820a,820b to charge the capacitors 830 when RF energy is not activated. Current associated with positive and negative voltage signals 820a,820b are provided through resistors 832 to charge capacitor 830. Diodes 833 are configured so that the capacitors are charged and discharged in stages. In other embodiments, the charge accumulator 803 is configured to charge all stages simultaneously.

In some instances, the charge accumulator 803 may be configured in stages, wherein electrical energy is stored in each stage, as represented by stages 1 through 16 shown in FIG. 28. The electrical energy can later be delivered as high voltage when RF energy is activated. For example, capacitors 830 are shown comprised of capacitor pairs—e.g., pair C1,C2; pair C3,C4, . . . pair C31,C32—with each pair referred to as being in a stage. Each pair of capacitors includes capacitor 830a associated with energy storage from the positive high voltage signal 820a received by the charge accumulator 803 at Point A, and another capacitor 830b associated with energy storage from the negative high voltage signal 820b received by the charge accumulator 803 at Point B. Point A and Point B in FIG. 28 correspond to charge accumulator 803's input lines 840 (when switch 831 is positioned accordingly), and further correspond to Point A and Point B in FIG. 9 and voltage converter 802's output lines.

Transistors 834a,834b are shown coupled to a capacitor 830a,830b, respectively. In some instances, as shown, transistors 834a,834b are bipolar junction transistors (BJT) used as switching devices. When turned on, transistor 834a is configured to provide a high voltage signal received from capacitor 830a to a positive high voltage rail at Point C. Similarly, when turned on, transistor 834b is configured to provide a negative high voltage signal received from capacitor 830b to a negative high voltage rail at Point D. It should be understood that transistors 834a,834b may also be configured to provide inverted voltage signals without compromising the underlying principles of the invention. Transistors 834a,834b are further configured to receive input signals that turn the transistor on and off. For example, transistors 834a are configured to receive signals B1-B16 at the respective base inputs of BJTs 834a to turn on the respective BJT. Similarly, transistors 834b are configured to receive signals B′1-B′16 at the respective base inputs of BJTs 834b to turn on the respective BJT.

Positive and negative high voltage output rails at Point C and Point D, respectively, are shown coupled to switches 835. Point C and Point D are also referred to herein as charge accumulator 803's output lines and output positive and negative high voltage signals 813a,813b, respectively, when the corresponding transistors are turned on.

The output lines of charge accumulator 803 are shown floating while the input lines of charge accumulator 803 are coupled to the output lines of voltage converter 802. Thus no RF energy provided to the plasma generator. Switches 831 and 835 are configured to switch when RF energy is activated so that charging is interrupted and accumulated charge is discharged. For example, when user activates RF energy by depressing an activation switch, for example, switches 831 for are switched such that the input lines go from the contacts coupling it to the voltage converter 802 to floating. Switches 835 for the output lines of the charge accumulator 803 are switched such that output lines go from floating to contacts coupling it to input lines of the RF signal generator 804. The switches 831 and 835 are returned to the positions shown after RF energy is delivered to the RF signal generator 804. In some instances, switches 831 and 835 are configured to switch independently. For example, after RF energy is activated, switch 835 may not switch back to the position shown in FIG. 10 until all RF energy is has been delivered to the plasma generator.

When RF energy is activated, the charge accumulator 803 is configured to discharge stored energy in each stage sequentially such that the energy from each stage is sequentially multiplexed to RF signal generator 804. The sequential rate of discharge of each stage may vary depending on desired application and design considerations. For example, each transistor pair 834a,834b in each stage may be configured to turn on when an activation voltage signal (e.g., B1-B16 and B′1-B′16) is applied to its base. In this way, an activation voltage signal may be applied to a pair of transistors 834a,834b in a first stage, and then subsequently to a pair of transistors 834a,834b in a second stage, and so on, until all stages have discharged.

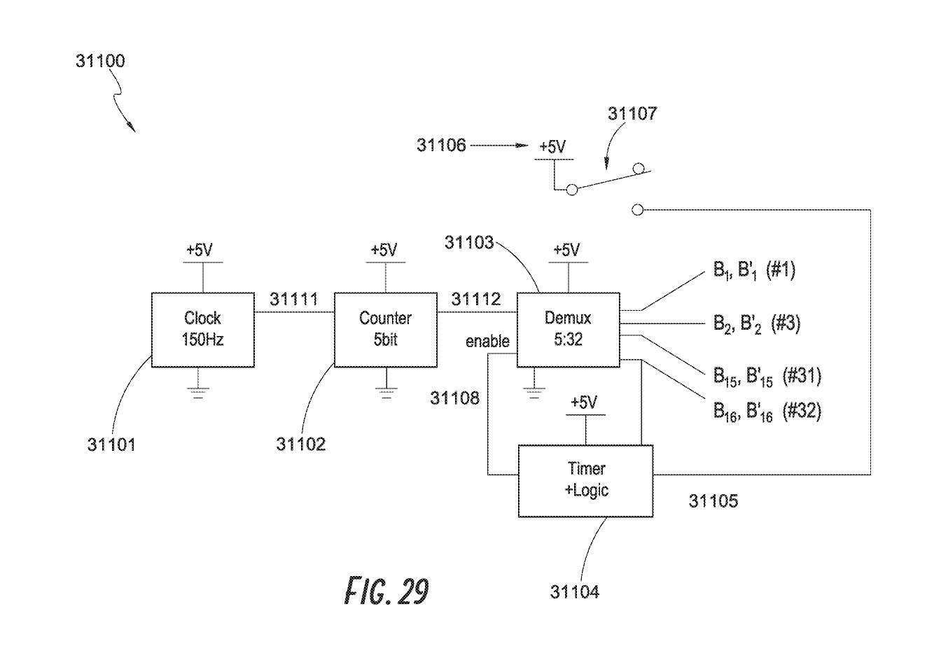

A modulation circuit (e.g., the one described in FIG. 29) may be implemented to provide the activation voltages signals sequentially to each stage at a modulated rate, as described further in FIG. 29. Thus, charge accumulator 803 receives a high voltage signal from voltage converter 802 and outputs a high voltage modulated signal on its output lines. The modulation rate can range from 1 Hz to 10 kHz, such as from 1 Hz to 500 Hz, and including from 10 Hz to 100 Hz. The duty cycle may also vary and range from 5% to 95%, such as from 25% to 75%, and including from 45% to 55%). In some embodiments, the duty cycle is approximately 50%.

FIG. 29 illustrates a functional block diagram of a modulation circuit 31100 coupled to charge accumulator 803 shown in FIG. 10, according to one embodiment. Modulation circuit 31100 is coupled to the charge accumulator 803 and outputs activation voltage signals (B1,B1′ to B16-B16′) to turn on the transistors 834a,834b in charge accumulator 803, thus discharging the stored charge in the pairs of capacitors 830a,830b at a modulated rate. More specifically, the activation voltage signals (B1,Bl′ to B16-816′) from the output of the modulation circuit 3110 are input into the base of the transistor and bias the transistor and turn it on and off accordingly.

In this embodiment, the modulation circuit 31000 comprises a clock source 31101 (e.g., 50 Hz clock as shown) coupled to a counter 31102 (e.g., 5 bit counter as shown). Counter 31102 receives a clock signal 31111 from the clock source 1101 and provides a counting output 31112 to demultiplexer 31103—e.g., a count corresponding to each clock cycle. Demultiplexer 31103 thus receives an incremental counting signal 31112 from the counter 31102 (e.g., at 50 Hz as shown). Demultiplexer 31103 is also coupled to a timer 31104 which enables and disables the demultiplexer 31103. Timer 31104 includes a input enable line 31105 which is floating until RF energy is activated (e.g., by user depressing an activation switch) at which point the input line 31105 is connected to a power source 31106 (e.g., 5 volts as shown) via switch 31107 to enable timer circuit 31104. Switch 31107 returns to its original position thereafter (e.g., after depression of the activation switch by user. Timer 31104 provides an enable signal 31108 to the demultiplexer 31103 for a predetermined amount of time.

Demultiplexer 31103 is shown having a plurality of output lines, each coupled to respective bases of transistor pairs 834a,834b in a given stage of the charge accumulator 803. Each capacitor pair of the set is coupled to the demultiplexer by a corresponding transistor. The demultiplexer 31103 is configured so that each output line (shown as #1 through #32 in FIG. 29) provides an activation voltage signal (shown as signals B1,B1′ through B16,B16′) at the occurrence of a corresponding count 31112 of the counter 31102 received while the demultiplexer is enabled. Thus, the count 31112 of the counter 31102 provides the rate at which the stages of capacitor pairs 834a,834b are discharged. For instance, when the demultiplexer 31103 is enabled by the timer 31104, the first count of the counter 31102 may correspond to activation voltage signals (B1 and B1′) being applied to the first output (line #1) for the demultiplexer 31103, which in turn turns on respective transistors 834a,834b and discharges respective capacitors 830a,830b in the first stage (stage 1) of the charge accumulator 803. The second count of the counter 31102 may correspond to an activation voltage signals (B2 and B2′) being applied to the second output (line #2) for the demultiplexer 31103, which in turn turns on respective transistors 834a,834b and discharges respective capacitors 830a,830b in the second stage (stage 2) of the charge accumulator 803. This continues until all counts corresponding to all output lines (lines 1-16) and discharging of all capacitors 830a,830b of in all stages (stages 1-16) of charge accumulator 803 have occurred. After all stages have been discharged, timer 31104 may be configured to disable the demultiplexer 31103. For example, timer 31104 may be configured to receive the last activation signal corresponding to the discharge of the last stage and disable upon receipt, thus disabling the demultiplexer.

The RF tissue modulation devices may be configured to deliver RF energy from the RF energy source to the plasma generator for a therapeutic duration. The therapeutic duration may range, for example, from minutes or less, including 30 seconds or less, such as 10 seconds or less. In some instances, the therapeutic duration may range from 1 to 2 seconds. The therapeutic duration may be controlled using a variety of implementations. For example, the RF tissue modulation device may be configured to return switches 831, 835, 31107 to their charging positions after a predetermined amount of time.

When switches 831, 835, 31107 are returned to charging positions, the charge accumulator 803 may once again store charge in the capacitors 830. In some instances, the RF tissue modulation device is configured to recharge the charge accumulator within a minimum recharge period between plasma generation. The minimum recharge period may range, for example, from 10 minutes or less, including 5 minutes or less, such as 3 minutes or less. In some instances, the minimum recharge period ranges from 1 to 2 minutes. Various recharge periods can be implemented by varying, for example, battery size, voltage boosting levels, and/or capacitance sizes.

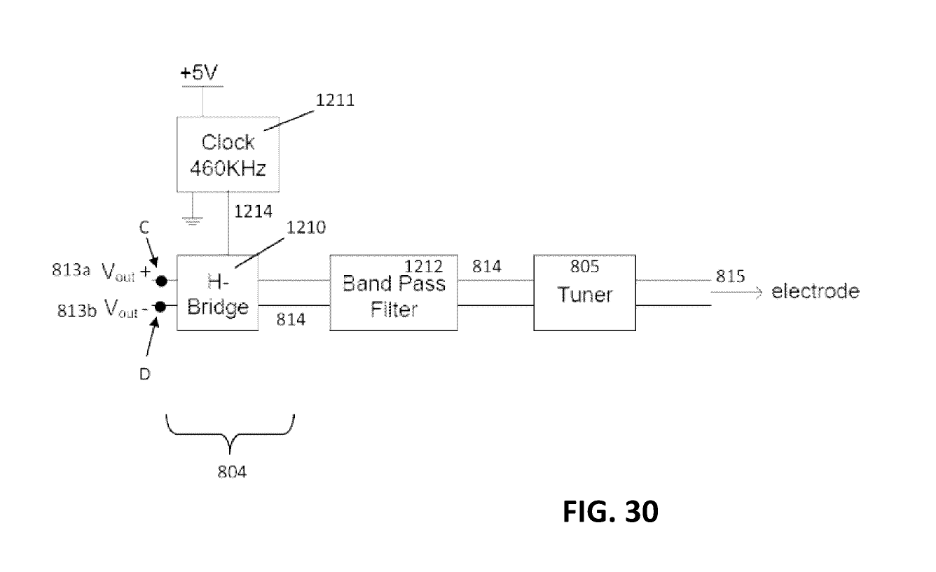

FIG. 30 illustrates a functional block diagram of an RF signal generator 804 and RF tuner 805 shown in FIG. 8, according to one embodiment. RF signal generator 804 outputs a high voltage modulated RF signal 814 at a specific operating frequency. In the embodiment shown, RF signal generator 804 includes an H-bridge 1210, an RF clock source 1211, and optional bandpass filter 1212. The H-bridge 1210 is coupled to the charge accumulator 803 and includes input lines at Point C and Point D that receive the positive and negative high voltage modulated signals 813a,813, respectively, provided by the positive and negative high voltage output rails at Point C and Point D, respectively, of charge accumulator 803 in FIG. 10 (when switch 835 is positioned accordingly).

H-bridge 1210 is coupled to an RF clock source 1211 and receives the positive and negative high voltage modulated signals 813a,813b at Point C and Point D. H-bridge 1210 switches the polarities of the positive and negative high voltage modulated signals 813a,813b based on an RF clock signal 1214 received by the RF clock source 1211, thus outputting a high voltage modulated RF signal 814. The switching provided at the output of the H-bridge 1210 is switched at an operating frequency based on the RF clock signal 1214. The operating frequency can range, for example, from 1 KHz to 50 MHz, such as from 100 KHz to 25 MHz, and including from 250 KHz to 10 MHz. In some embodiments, the RF voltage signal is a sine wave with operating frequency 460 kHz.

The resulting high voltage modulated RF signal 814 is provided to the plasma generator and provides the necessary power and voltage to generate a plasma. An optional bandpass filter 1212 is shown coupled to H-bridge 1210 and filters the signal to eliminate noise and output it to optional RF tuner 805. RF tuner 805 receives the high voltage modulated RF signal 814 and outputs a tuned high voltage modulated RF signal 815 as described above for FIG. 24.

### Further Embodiments of Methods

Aspects of the subject invention also include methods of modifying an internal target tissue of a subject. In certain embodiments, the methods of modifying an internal target site include positioning the distal end of a minimally invasive RF tissue modulation device at a target tissue site. In some instances, the RF tissue modulation device may comprise a hand-held control unit and RF probe, as described above. In some instances, the RF tissue modulation device may include an RF probe, medical device, and adapter operably coupled to the medical device.

The methods further include activating RF energy for delivery to a plasma generator at a distal end of the minimally invasive RF tissue modulation device. Still further, the methods include generating RF energy, delivering the RF energy to the plasma generator, and generating a plasma at the plasma generator to deliver RF energy to the internal target tissue site of the subject. For example, a plasma may be generated between an RF electrode of the plasma generator and the outer surface of the elongated member, resulting in tissue modification. In some instances, irrigating conducting fluid is provided. In some instances, the plasma generator may further be translated and/or rotated while supplying RF energy (and irrigating conducting fluid in some instances)—e.g, resulting in tissue dissection. In some instances, the entire end of the RF tissue modulation device may be translated proximally and distally until the desired tissue dissection is obtained. When finished with tissue dissection at the first location, the device may be rotated 180 degrees and further tissue removed using the steps described above.

Aspects of the subject invention may also include methods of generating RF energy for delivery to an internal target tissue of a subject. In some embodiments, the methods of generating RF energy include providing electrical energy from an electrical energy source to a charge accumulator, and storing energy in a charge accumulator. The methods may further include discharging the electrical energy to an RF signal generator and generating a modulated RF signal output. The methods may further include boosting the voltage of the modulated RF signal using a voltage converter to generate a high voltage modulated RF signal. In some instances, the methods further include providing the high voltage modulated RF signal to an RF tuner and outputting a tuned high voltage RF signal to a plasma generator.

In some embodiments, the methods of generating RF energy include providing electrical energy from an electrical energy source to a charge accumulator, storing energy in a charge accumulator, and discharging the electrical energy to voltage converter. The methods may further include providing an RF clock signal from an RF clock source to the voltage converter and generating a modulated high voltage signal output. The methods may further include providing the modulated high voltage signal to an RF signal generator to generate a high voltage modulated RF signal output. In some instances, the methods further include providing the high voltage modulated RF signal to an RF tuner and outputting a tuned high voltage RF signal to a plasma generator.

In some embodiments, the methods of generating RF energy include providing electrical energy from an electrical energy source to a voltage converter. The methods further include generating a high voltage positive and negative voltage, providing the high voltage positive and negative voltage to a charge accumulator, storing energy within the charge accumulator, and discharging positive and negative high voltage modulated signals from the charge accumulator. In some instances, the discharging of positive and negative high voltage modulated signals may include activating a modulation circuit to discharge the charge accumulator in stages at a modulated rate. The methods may further include providing the positive and negative high voltage modulated signals from the charge accumulator to an H-bridge operating at a frequency based on an RF clock signal to generate positive and negative high voltage modulated RF signal outputs. In some instances, the methods further include providing the high voltage modulated RF signal to an RF tuner and outputting a tuned high voltage RF signal to a plasma generator.

Aspects of the invention further include methods of imaging an internal tissue site with RF tissue modulation devices of the invention. A variety of internal tissue sites can be modified and/or imaged with devices of the invention. In certain embodiments, the methods are methods of imaging an intervertebral disc in a minimally invasive manner. For ease of description, the methods are now primarily described further in terms of imaging IVD target tissue sites. However, the invention is not so limited, as the devices may be used to image a variety of distinct target tissue sites.

With respect to imaging an intervertebral disc or portion thereof, e.g., exterior of the disc, nucleus pulposus, etc., embodiments of such methods include positioning a distal end of an RF tissue modulation device of the invention in viewing relationship to an intervertebral disc or portion of there, e.g., nucleus pulposus, internal site of nucleus pulposus, etc. By viewing relationship is meant that the distal end is positioned within 40 mm, such as within 10 mm, including within 5 mm of the target tissue site of interest. Positioning the distal end of the RF tissue modulation device in relation to the desired target tissue may be accomplished using any convenient approach, including through use of an access device, such as a cannula or retractor tube, which may or may not be fitted with a trocar, as desired.

Methods of invention may include visualizing the internal target tissue site via a visualization sensor integrated at the distal end of the elongated member of the RF tissue modulation device. The visualizing may include obtaining image data of an internal tissue site with the visualization sensor and then forwarding the image data to an image processing module of a system of the invention. Methods of invention may also include receiving image data into a system that includes an image processing module of the invention. The methods may further include viewing an image produced from the image data received by the image processing module. In some instances, the methods include visualizing the internal target tissue via a remote monitor.

Methods of the invention may further include illuminating the internal target tissue site via an illuminator integrated at the distal end of the elongated member. For example, following positioning of the distal end of the RF tissue modulation device in viewing relationship to the target tissue, the target tissue, e.g., intervertebral disc or portion thereof, is imaged through use of the illumination and visualization elements to obtain image data. Image data obtained according to the methods of the invention is output to a user in the form of an image, e.g., using a monitor or other convenient medium as a display means. In certain embodiments, the image is a still image, while in other embodiments the image may be a video.

In certain embodiments, the methods include a step of tissue modification using RF energy, as described in the methods above. For example, the methods may include a step of tissue removal using RF energy, e.g., using a combination of tissue cutting and irrigation or flushing. For example, the methods may include cutting a least a portion of the tissue using RF energy and then removing the cut tissue from the site, e.g., by flushing at least a portion of the imaged tissue location using a fluid introduced by an irrigation lumen and removed by an aspiration lumen.

The internal target tissue site may vary widely. Internal target tissue sites of interest include, but are not limited to, cardiac locations, vascular locations, orthopedic joints, central nervous system locations, etc. In certain cases, the internal target tissue site comprises spinal tissue.

The subject methods are suitable for use with a variety of mammals. Mammals of interest include, but are not limited to: race animals, e.g. horses, dogs, etc., work animals, e.g. horses, oxen etc., and humans. In some embodiments, the mammals on which the subject methods are practiced are humans.

Aspects of the invention further include methods of assembling an RF tissue modulation device. In these embodiments, the methods include operably coupling a proximal end of an elongated member to a hand-held control unit, e.g., as described above. Depending on the particular configuration, this step of operably coupling may include a variety of different actions, such as snapping the elongated member into a receiving structure of the hand-held control unit, twist locking the elongated member into a receiving structure of the hand-held control unit, and the like. In some instances, methods of assembling may further include sealing the hand-held control unit inside of a removable sterile covering, where the sterile covering is attached to the proximal end of the elongated member and configured to seal the hand-held control unit from the environment, e.g., as described above. In such instances, the methods may further include sealing a proximal end of the sterile covering.

In some embodiments, the methods of assembly include operably coupling a proximal end of an adapter to a hand-held medical device, e.g., a visualization device as described above. In some instances, the medical device includes a removable section that is removed before the adapter may be operably coupled. Depending on the particular configuration, this step of operably coupling may include a variety of different attachment mechanisms, such as snapping, hinging, using magnetics, etc. In some instances, medical device does not include a removable section that is required to be removed before operably coupling adapter to the medical device.

In some instances, methods of assembling may further include sealing the hand-held control unit inside of a removable sterile covering, where the sterile covering is attached to the proximal end of the elongated member and configured to seal the hand-held control unit from the environment, e.g., as described above. In such instances, the methods may further include sealing a proximal end of the sterile covering.

### Further Examples of Utility

The subject RF tissue modulation devices and methods find use in a variety of different applications where it is desirable to modify (and image, in some instances) an internal target tissue of a subject while minimizing damage to the surrounding tissue.

The subject devices and methods find use in many applications, such as but not limited to surgical procedures, that involve for example, removing small amounts of tissue via RF resection, RF ablation of a minor surface region of tissue, or coagulation of a limited area of exposed blood vessels, etc. Such surgical fields may include, for example, sports medicine, orthopedics, arthroscopy, spine surgery, laparoscopy, END, and neurosurgery. Example applications may include, for instance, debriding a torn meniscus, performing a micro-discectomy on a herniated lumbar disc, treating carpal tunnel syndrome by severing tissue around the nerve, etc.

The subject devices and methods find use in many applications, such as but not limited to surgical procedures, where a variety of different types of tissues may be removed, including but not limited to: soft tissue, cartilage, bone, ligament, etc. Specific procedures of interest include, but are not limited to, spinal fusion (such as Transforaminal Lumbar Interbody Fusion (TLIF)), total disc replacement (TDR), partial disc replacement (PDR), procedures in which all or part of the nucleus pulposus is removed from the intervertebral disc (IVD) space, arthroplasty, and the like. As such, methods of the invention also include treatment methods, e.g., where a disc is modified in some manner to treat an existing medical condition. Treatment methods of interest include, but are not limited to: annulotomy, nucleotomy, discectomy, annulus replacement, nucleus replacement, and decompression due to a bulging or extruded disc. Additional methods in which the RF tissue modulation devices may find use include those described in United States Published Application No. 20080255563.

In certain embodiments, the subject devices and methods facilitate the dissection of the nucleus pulposus while minimizing thermal damage to the surrounding tissue. In addition, the subject devices and methods can facilitate the surgeon's accessibility to the entire region interior to the outer shell, or annulus, of the IVD, while minimizing the risk of cutting or otherwise causing damage to the annulus or other adjacent structures (such as nerve roots) in the process of dissecting and removing the nucleus pulposus.

Furthermore, the subject devices and methods may find use in other procedures, such as but not limited to ablation procedures, including high-intensity focused ultrasound (HIFU) surgical ablation, cardiac tissue ablation, neoplastic tissue ablation (e.g. carcinoma tissue ablation, sarcoma tissue ablation, etc.), microwave ablation procedures, and the like. Yet additional applications of interest include, but are not limited to: orthopedic applications, e.g., fracture repair, bone remodeling, etc., sports medicine applications, e.g., ligament repair, cartilage removal, etc., neurosurgical applications, and the like.

### Further Embodiments of Kits

Also provided are kits for use in practicing the subject methods, where the kits may include one or more of the above devices, and/or components thereof, e.g., elongated members (RF probes), hand-held control units, adapters, sterile coverings, etc., as described above. For example, the kits may include one or more of the following: a hand-held device as described above, an adapter as described above, an RF probe as described above, and other types of probes, such as a visualization probe. The kits may further include other components, e.g., guidewires, access devices, fluid sources, etc., which may find use in practicing the subject methods. Various components may be packaged as desired, e.g., together or separately.

In addition to above mentioned components, the subject kits may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

It should be understood that some of the techniques introduced above can be implemented by programmable circuitry programmed or configured by software and/or firmware, or they can be implemented entirely by special-purpose “hardwired” circuitry, or in a combination of such forms. Such special-purpose circuitry (if any) can be in the form of, for example, one or more application-specific integrated circuits (ASICS), programmable logic devices (PLDs), field-programmable gate arrays (FPGAs), etc. For example, various switches, timers, etc., may be implemented in software and/or firmware, or they can be implemented entirely by special-purpose “hardwired” circuitry.

Software or firmware implementing the techniques introduced herein may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing took, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc. The term “logic”, as used herein, can include, for example, special purpose hardwired circuitry, software and/or firmware in conjunction with programmable circuitry, or a combination thereof. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

Further embodiments can be seen in the following paragraphs:

1. A minimally invasive tissue modification system, the system comprising:
(a) a minimally invasive access device having a proximal end, a distal end and an internal passageway; and
(b) an elongated tissue modification device having a proximal end and a distal end, wherein the tissue modification device is dimensioned to be slidably moved through the internal passageway of the access device;
wherein the system includes an illumination element and a visualization element positioned among the distal ends of the access device and tissue modification device.

2. The minimally invasive tissue modification system according to Claim 1, wherein the illumination element comprises a LED.

3. The minimally invasive tissue modification system according to Claim 1, wherein the illumination element comprises a fiber optic light source.

4. The minimally invasive tissue modification system according to Claim 1, wherein the illumination element comprises both a LED and a fiber optic light source.

5. The minimally invasive tissue modification system according to Claim 1, wherein the illumination element includes a diffusion element.

6. The minimally invasive tissue modification system according to Claim 1, wherein the visualization element is selected from a CCD and a CMOS sensor.

7. The minimally invasive tissue modification system according to Claim 6, wherein the visualization element is operably coupled to an image display unit at the proximal end of the tissue modification device.

8. The minimally invasive tissue modification system according to Claim 1, wherein the tissue modifier is a mechanical tissue modifier.

9. The minimally invasive tissue modification system according to Claim 8, wherein the tissue modification device is a rongeur.

10. The minimally invasive tissue modification system according to Claim 9, wherein the visualization element is positioned at the distal tip of the rongeur.

11. A method of modifying an internal target tissue of a patient, the method comprising:
(a) positioning a minimally invasive access device having a proximal end, a distal end and an internal passageway so that the distal end is near the target tissue, wherein the distal end comprises an illumination element; and
(b) slidably moving an elongated tissue modification device having a proximal and distal end through the internal passageway of the access device so that the distal end is operably positioned in relation to the target tissue, wherein the tissue modification device includes a tissue modifier and a visualization element integrated at the distal end; and
(c) modifying the target tissue with the tissue modifier.

12. The method according to Claim 11, wherein the illumination element comprises a LED and the method comprises illuminating the target tissue with the LED.

13. The method according to Claim 11, wherein the illumination element comprises a fiber optic light source and the method comprises illuminating the target tissue with the fiber optic light source.

14. The method according to Claim 11, wherein the illumination element comprises both a LED and a fiber optic light source and the method comprising illuminating the target tissue with both the LED and the fiber optic light source.

15. The method according to Claim 11, wherein the visualization element is selected from a CCD and a CMOS sensor and the method comprising obtaining one or more image frames of the target tissue with the visualization element.

16. The method according to Claim 15, wherein the visualization element is operably coupled to an image display unit at the proximal end of the tissue modification device and the method comprises viewing the obtained one or more image frames on the image display unit.

17. The method according to Claim 11, wherein the tissue modifier is a tissue remover and the method comprises removing tissue with the tissue remover.

18. The method according to Claim 17, wherein the tissue modification device is a rongeur.

19. The method according to Claim 18, wherein the visualization element is integrated with the forceps of the rongeur.

20. The method according to Claim 19, wherein the target tissue is spinal tissue.

21. The method according to Claim 20, wherein the method is a method of removing nucleus pulposus tissue from a herniated intervertebral disc.

22. A kit comprising:
(a) a minimally invasive access device having a proximal end, a distal end and an internal passageway, wherein the distal end comprises an illumination element; and
(b) an elongated tissue modification device having a proximal end and a distal end, wherein the tissue modification is dimensioned to be slidably moved through the internal passageway of the access device and includes a visualization element at the distal end.

23. The kit according to Claim 22, wherein the illumination element comprises a LED.

24. The kit according to Claim 22, wherein the illumination element comprises a fiber optic light source.

25. The kit according to Claim 22, wherein the illumination element comprises both a LED and a fiber optic light source.

26. The kit according to Claim 22, wherein the illumination element comprises a diffusion element.

27. The kit according to Claim 22, wherein the visualization element is selected from a CCD and a CMOS sensor.

28. The kit according to Claim 27, wherein the visualization element is operably coupled to an image display unit at the proximal end of the tissue modification device.

29. The kit according to Claim 22, wherein the tissue modifier is a tissue remover.

30. The kit according to Claim 29, wherein the tissue modification device is a rongeur.

31. The kit according to Claim 30, wherein the visualization element is integrated at the distal tip of the rongeur.

32. A minimally invasive access device having a proximal end, a distal end and an internal passageway, wherein the distal end comprises an illumination element.

33. The minimally invasive access device according to Claim 32, wherein the illumination element comprises a LED.

34. The minimally invasive access device according to Claim 32, wherein the illumination element comprises a fiber optic light source.

35. The minimally invasive access device according to Claim 32, wherein the illumination element comprises both a LED and a fiber optic light source.

36. The minimally invasive access device according to Claim 32, wherein the illumination element includes a diffusion element.

37. An internal tissue visualization system, the system comprising:
(a) an internal tissue visualization device comprising:
(i) an elongated member having a proximal end and a distal end; and
(ii) an RF-shielded visualization sensor module; and
(b) an extra-corporeal control unit operatively coupled to the proximal end of the elongated member.

38. The system according to claim 37, wherein the RF-shielded visualization sensor module comprises a:
a visualization sensor comprising a lens and an integrated circuit, wherein the visualization sensor is integrated at the distal end of the elongated member; and
a grounded conductive enclosure that shields the integrated circuit from an RF field.

39. The tissue modification device according to claim 38, wherein the visualization sensor is a CMOS device.

40. The tissue modification device according to claim 38, wherein the visualization sensor is a CCD device.

41. The system according to claim 38, wherein the grounded conductive enclosure comprises a housing comprising an outer grounded conductive layer.

42. The system according to claim 41, wherein the outer grounded conductive layer is a metallic layer.

43. The system according to claim 38, wherein the RF-shielded visualization sensor module further comprises an RF-shielded conductive member that connects the visualization sensor to a proximal end location of the elongated member.

44. The system according to claim 37, wherein the distal end of the elongated member further comprises an integrated illuminator.

45. The system according to claim 44, wherein the illuminator is a light emitting diode.

46. The system according to claim 45, wherein the RF-shielded visualization sensor module comprises the light emitting diode.

47. The system according to claim 37, wherein the system further comprises a tissue modifier at the distal end of the elongated member.

48. The system according to claim 43, wherein the tissue modifier is integrated at the distal end.

49. The system according to claim 43, wherein the tissue modifier comprises an electrode.

50. The system according to claim 37, wherein the system comprises an image displayer for displaying to a user images obtained by the visualization sensor.

51. An internal tissue visualization device comprising: an elongated member having a proximal end and a distal end; and an RF-shielded visualization sensor module.

52. The device according to claim 51, wherein the RF-shielded visualization sensor module comprises:
a visualization sensor comprising a lens and an integrated circuit, wherein the visualization sensor is integrated at the distal end of the elongated member; and
a grounded conductive enclosure that shields the integrated circuit from an RF field.

53. The device according to claim 52, wherein the visualization sensor is a CMOS device.

54. The device according to claim 52, wherein the visualization sensor is a CCD device.

55. The device according to claim 52, wherein the grounded conductive enclosure comprises a housing comprising an outer grounded conductive layer.

56. The device according to claim 52, wherein the RF-shielded visualization sensor module further comprises an RF-shielded conductive member that connects the visualization sensor to a proximal end location of the elongated member.

57. The device according to claim 51, wherein the distal end of the elongated member further comprises an integrated illuminator.

58. The device according to claim 51, wherein the system further comprises a tissue modifier at the distal end of the elongated member.

59. The device according to claim 58, wherein the tissue modifier comprises an electrode.

60. A method of imaging an internal target tissue site of a subject, the method comprising:
(a) positioning the distal end of an internal tissue visualization device comprising:
(i) an elongated member having a proximal end and a distal end; and
(ii) an RF-shielded visualization sensor module;
in operable relation to the internal target tissue site; and
(b) visualizing the internal target tissue site with the RF-shielded visualization sensor module.

61. The method according to claim 60, wherein the internal target tissue site comprises spinal tissue.

62. The method according to claim 61, wherein the device further comprises a distal end tissue modifier and the method further comprises modifying tissue with the tissue modifier.

63. An internal tissue visualization device, the device comprising:
(a) a hand-held control unit comprising a monitor; and
(b) an elongated member having a proximal end operatively coupled to the hand-held control unit and a minimally-dimensioned distal end having an integrated visualization sensor.

64. The device according to claim 63, wherein the minimally dimensioned distal end has an outer diameter that is 5 mm or less.

65. The device according to claim 64, wherein the minimally dimensioned distal end has an outer diameter that is 3 mm or less.

66. The device according to claim 63, wherein the integrated visualization sensor comprises a CMOS device.

67. The device according to claim 63, wherein the distal end of the elongated member further comprises an integrated illuminator.

68. The device according to claim 67, wherein the integrated illuminator comprises a configuration selected from the group consisting of a crescent configuration and a concentric configuration.

69. The device according to claim 63, wherein the elongated member comprises an annular wall configured to conduct light to the elongated member distal end from a proximal end source.

70. The device according to 69, wherein the proximal end source comprises a forward focused light emitting diode.

71. The device according to claim 70, wherein the forward focused light emitting diode is configured to direct light along the outer surface of the elongated member.

72. The device according to claim 63, wherein the elongated member comprises a fluid filled structure configured to conduct light to the elongated member distal end from a proximal end source.

73. The device according to 72, wherein the proximal end source comprises a forward focused light emitting diode.

74. The device according to claim 73, wherein the forward focused light emitting diode is configured to direct light along the outer surface of the elongated member.

75. The device according to claim 67, wherein the device is configured to reduce coupling of light directly from the integrated illuminator to the visualization sensor.

76. The device according to claim 76, wherein the device comprises a distal end polarized member.

77. The device according to claim 76, wherein the polarized member polarizes light from the integrated illuminator.

78. The device according to claim 76, wherein the polarized member filters light reaching the visualization sensor.

79. The device according to claim 63, wherein the proximal end of the elongated member is configured to be detachable from the hand-held control unit.

80. The device according to claim 79, wherein the device comprises a removable sterile covering attached to the proximal end of the elongated member that is configured to seal the hand-held control unit from the environment.

81. The device according to claim 80, wherein the hand-held control unit comprises a handle portion and a controller.

82. The device according to claim 81, wherein the sterile covering comprises a window portion configured to associate with the monitor and boot portion configured to associated with the controller.

83. The device according to claim 82, wherein the window portion is configured to provide for touch screen interaction with the monitor.

84. The device according to claim 83, wherein the sterile covering comprises a seal at a region associated with the proximal end of the hand-held control unit.

85. The device according to claim 63, wherein the monitor is configured to communicate wirelessly with another device.

86. The device according to claim 85, wherein the monitor is configured to be detachable from the hand-held control unit.

87. The device according to claim 63, wherein the elongated member comprises a distal end integrated non-visualization sensor.

88. The device according to claim 87, wherein the distal end integrated non-visualization sensor is a sensor selected from the group consisting of: temperature sensors, pressure sensors, pH sensors, impedance sensors, conductivity sensors and elasticity sensors.

89. The device according to claim 87, wherein the sensor is deployable.

90. The device according to claim 63, wherein the elongated member comprises a lumen that extends for at least a portion of the elongated member.

91. The device according to claim 63, wherein the distal end of the elongated member comprises a tool selected from the group consisting of a low-profile biopsy tool and a low-profile cutting tool

92. The device according to claim 91, wherein the low-profile biopsy tool comprises an annular cutting member concentrically disposed about the distal end of the elongated member and configured to be moved relative to the distal end of the elongated member in a manner sufficient to engage tissue.

93. The device according to claim 63, wherein the integrated visualization sensor comprises an RF-shielded visualization module.

94. The device according to claim 63, wherein the elongated member is configured for distal end articulation.

95. The device according to claim 63, wherein the device comprises a stereoscopic image module.

96. The device according to claim 63, wherein the device comprises an image recognition module.

97. The device according to claim 63, wherein the device comprises a collimated laser.

98. A method of imaging an internal target tissue site of a subject, the method comprising:
(a) positioning the distal end of an internal tissue visualization device in operable relation to the internal target tissue site, where the device comprises:
(i) a hand-held control unit comprising a monitor; and
(ii) an elongated member having a proximal end operatively coupled to the hand-held control unit and a minimally-dimensioned distal end having an integrated visualization sensor; and
(b) visualizing the internal target tissue site with the visualization sensor.

99. The method according to claim 98, wherein the internal target tissue site comprises spinal tissue.

100. The method according to claim 99, wherein the device further comprises a distal end low-profile biopsy tool and the method further comprises obtaining a tissue biopsy with the low-profile biopsy tool.

101. A method of assembling an internal tissue visualization device, the method comprising operatively coupling a proximal end of an elongated member to a hand-held control unit, wherein the elongated member comprises a distal end integrated visualization sensor and the hand-held control unit comprises a monitor.

102. The method according to claim 101, wherein the method further comprises sealing the hand-held control unit inside of a removable sterile covering attached to the proximal end of the elongated member and configured to seal the hand-held control unit from the environment.

103. The method according to claim 102, wherein the hand-held control unit comprises a handle portion and a controller and the sterile covering comprises a window portion configured to associate with the monitor and boot portion configured to associated with the manual controller.

104. The method according to claim 103, wherein the method comprises sealing a proximal end of the sterile covering.

105. A minimally invasive RF tissue modulation device, the device comprising:
(a) a hand-held control unit comprising an electrical energy source; and
(b) an elongated member having a proximal end operably coupled to the hand-held control unit and a minimally-dimensioned distal end comprising a plasma generator;
wherein the device is configured to generate a plasma at the plasma generator for a therapeutic duration.

106. The device according to Claim 105, wherein the device comprises a voltage converter, a charge accumulator and an RF signal generator collectively operably coupling the electrical energy source to the plasma generator.

107. The device according to Claim 106, wherein the RF signal generator comprises a power amplifier and an RF clock source.

108. The device according to Claim 107, wherein the RF signal generator is configured to receive a first signal from the charge accumulator and to output a second signal to the voltage converter.

109. The device according to Claim 108, wherein the RF signal generator is configured to receive a clock signal from a second clock source and to output the second signal as a modulated signal based on the clock signal.

110. The device according to Claim 107, wherein the voltage converter is configured to receive a first signal from the charge accumulator and to output a second signal to the RF signal generator.

111. The device according to Claim 110, wherein the voltage converter is configured to receive a clock signal and to output the second signal as a modulated signal based on the clock signal.

112. The device according to Claim 107, wherein the charge accumulator is configured to receive a first signal from the voltage converter and to output a second signal to the RF signal generator.

113. The device according to Claim 105, wherein the electrical energy source comprises one or more batteries.

114. The device according to Claim 106, wherein the voltage converter is a DC to DC converter.

115. The device according to Claim 106, wherein the charge accumulator comprises a single capacitor.

116. The device according to Claim 106, wherein the charge accumulator comprises a set of two or more capacitor pairs.

117. The device according to Claim 116, wherein the device comprises a demultiplexer configured to produce a modulated signal output from the set.

118. The device according to Claim 117, wherein each capacitor pair of the set is coupled to the demultiplexer by a transistor.

119. The device according to Claim 118, wherein the transistor is a bipolar junction transistor.

120. The device according to Claim 107, wherein the RF signal generator comprises a H-bridge.

121. The device according to Claim 105, further comprising a band pass filter.

122. The device according to Claim 105, further comprising a tuner.

123. The device according to Claim 105, wherein the therapeutic duration is 1 second or longer.

124. The device according to Claim 123, wherein the therapeutic duration ranges from 1 to 2 seconds.

125. The device according to Claim 105, wherein the device is configured to have a minimum recharge period between plasma generation.

126. The device according to Claim 125, wherein the recharge period ranges from 1 to 2 minutes.

127. The device according to Claim 105, wherein the plasma generator is configured to produce a plasma are between a first conductive member positioned inside of the distal end of the elongated member and an outer surface of the elongated member.

128. The device according to Claim 105, wherein the elongated member comprises a distal end opening positioned over the first conductive member.

129. The device according to Claim 127, wherein the first conductive member is coupled to an RF line adjacent to an RF shield within the elongated member.

130. The device according to Claim 127, wherein the first conductive member is positioned within a distal end opening of the elongated member by an insulator.

131. The device according to Claim 130, wherein the insulator is ceramic.

132. The device according to Claim 105, wherein the plasma generator is configured to produce a plasma arc between a first conductive member positioned substantially at a tip of the elongated member and an outer surface of the elongated member.

133. The device according to Claim 105, wherein the elongated member comprises a distal end integrated visualization sensor and the device further comprises a monitor.

134. The device according to Claim 106, wherein the voltage converter, charge accumulator and RF signal generator are present inside of the hand-held control unit.

135. The device according to Claim 106, wherein the voltage converter, charge accumulator and RF signal generator are present in an adapter configured to be attached to the hand-held control unit during use.

136. The device according to Claim 105, wherein the elongated member is configured to be detachable from the hand-held control unit.

137. A method of delivering RF energy to an internal target tissue site of a subject, the method comprising:
(a) positioning the distal end of an elongated member of a device according to Claim 1 at the internal target tissue site of a subject; and
(b) generating a plasma from the plasma generator to deliver RF energy to the internal target tissue site of the subject.

138. The method according to Claim 137, further comprising visualizing the internal target tissue site via a visualization sensor integrated at the distal end of the elongated member.

139. The method according to Claim 138, further comprising illuminating the internal target tissue site via an illuminator integrated at the distal end of the elongated member.

140. The method according to Claim 137, further comprising visualizing the internal target tissue site via a remote monitor.

141. The method according to Claim 140, wherein the device and the remote monitor communicate wirelessly.

142. An adapter comprising:
an electrical energy source; and
a voltage converter;
a charge accumulator; and
an RF signal generator.

143. The adapter according to Claim 142, wherein the adapter is configured to removably couple to a hand-held minimally dimensioned medical device.

144. The adapter according to Claim 143, wherein the adapter is configured to removably couple to the hand-held minimally dimensioned medical device in a manner such that it is positioned below the hand-held minimally dimensioned medical device when coupled thereto.

145. The adapter according to Claim 142, wherein the RF signal generator comprises a power amplifier and an RF clock source.

146. The adapter according to Claim 145, wherein the RF signal generator is configured to receive a first signal from the charge accumulator and to output a second signal to the voltage converter.

147. The adapter according to Claim 146, wherein the RF signal generator is configured to receive a clock signal from a second clock source and to output the second signal as a modulated signal based on the clock signal.

148. The adapter according to Claim 145, wherein the voltage converter is configured to receive a first signal from the charge accumulator and to output a second signal to the RF signal generator.

149. The adapter according to Claim 148, wherein the voltage converter is configured to receive a clock signal and to output the second signal as a modulated signal based on the clock signal.

150. The adapter according to Claim 145, wherein the charge accumulator is configured to receive a first signal from the voltage converter and to output a second signal to the RF signal generator.

151. The adapter according to Claim 142, wherein the electrical energy source comprises one or more batteries.

152. The adapter according to Claim 142, wherein the voltage converter is a DC to DC converter.

153. The adapter according to Claim 142, wherein the charge accumulator comprises a single capacitor.

154. The adapter according to Claim 142, wherein the charge accumulator comprises a set of two or more capacitor pairs.

155. The adapter according to Claim 154, wherein the adapter comprises a demultiplexer configured to produce a modulated signal output from the set.

156. The adapter according to Claim 155, wherein each capacitor pair of the set is coupled to the demultiplexer by a transistor.

157. The adapter according to Claim 156, wherein the transistor is a bipolar junction transistor.

158. The adapter according to Claim 145, wherein the RF signal generator comprises a H-bridge.

159. The adapter according to Claim 142, further comprising a band pass filter.

160. The adapter according to Claim 142, further comprising a tuner.

161. An RF probe comprising an elongated member configured to operably couple to a hand-held device at a proximal end of the elongated member, wherein a minimally-dimensioned distal end of the elongated member comprises a plasma generator.

162. The RF probe according to Claim 161, wherein the elongated member comprises a first conductive member positioned substantially at a tip of the elongated member.

163. The RF probe according to Claim 161, wherein the elongated member comprises a distal end opening positioned over the first conductive member.

164. The RF probe according to Claim 162, wherein the first conductive member is coupled to an RF line adjacent to an RF shield within the elongated member.

165. The RF probe according to Claim 162, wherein the first conductive member is positioned within a distal end opening of the elongated member by an insulator.

166. The RF probe according to Claim 165, wherein the insulator is ceramic.

167. The RF probe according to Claim 161, wherein the elongated member further comprises a distal end integrated visualization sensor.

168. A hand-held minimally dimensioned device configured to operably couple to an adapter according to Claim 38 and an RF probe according to Claim 161.

169. The device according to Claim 168, wherein the hand-held minimally dimensioned device comprises a monitor.

170. A kit comprising:
a set of components selected from a group consisting of:
(a) a hand-held device according to Claim 168, an adapter according to Claim 38 and an RF probe according to Claim 161;
(b) an RF probe according to Claim 161 and an adapter according to Claim 38; and
(c) an RF probe according to Claim 161 and a second visualization probe.

171. The kit according to Claim 170, wherein the adapter is configured to removably couple to the device.

172. The kit according to Claim 171, wherein the RF signal generator comprises a power amplifier and an RF clock source.

173. The kit according to Claim 172, wherein the RF signal generator is configured to receive a first signal from the charge accumulator and to output a second signal to the voltage converter.

174. The kit according to Claim 172, wherein the voltage converter is configured to receive a first signal from the charge accumulator and to output a second signal to the RF signal generator.

175. The kit according to Claim 172, wherein the charge accumulator is configured to receive a first signal from the voltage converter and to output a second signal to the RF signal generator.

176. The kit according to Claim 171, wherein the RF probe comprises a first conductive member positioned substantially at a tip of the elongated member.

177. The kit according to Claim 171, wherein the RF probe comprises a distal end opening positioned over the first conductive member.

178. The kit according to Claim 170, wherein the kit comprises a hand-held device according to Claim 64, an adapter according to Claim 38 and an RF probe according to Claim 57.

179. The kit according to Claim 170, wherein the kit comprises an RF probe according to Claim 161 and an adapter according to Claim 142.

180. The kit according to Claim 170, wherein the kit comprises an RF probe according to Claim 161 and a second visualization probe.

As described elsewhere in the specification, aspects of the invention include minimally invasive tissue modification systems. Embodiments of the systems include a minimally invasive access device having a proximal end, a distal end and an internal passageway. Also part of the system is an elongated tissue modification device having a proximal end and a distal end. The tissue modification device is dimensioned to be slidably moved through the internal passageway of the access device. The tissue modification device includes a tissue modifier. Positioned among the distal ends of the devices are a visualization element and an illumination element. Also provided are methods of using the systems in tissue modification applications, as well as kits for practicing the methods of the invention. Internal tissue visualization devices having RF-shielded visualization sensor modules are provided. Also provided are systems that include the devices, as well as methods of visualizing internal tissue of a subject using the tissue visualization devices and systems. Minimally invasive RF tissue modulation devices are provided. In some aspects, the devices include a hand-held control unit and an elongated member. The hand-held control unit includes an electrical energy source and the elongated member has a proximal end operably coupling to the hand-held control unit. The RF tissue modulation device is configured to generate a plasma at a distal end plasma generator for a therapeutic duration. In some aspects, RF tissue modulation devices are provided and include an adapter that operably couples to a hand-held medical device. The adapter generates RF energy for delivery to a plasma generator on an elongated member. Methods of delivering the RF energy to the internal target tissue site are also provided.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1-20. (canceled)

21. A tissue visualization and modification device comprising:
an elongated member, extending along a longitudinal axis between a proximal end affixed to a handpiece and a distal end, the distal end comprising a visualization sensor;
a tissue modifier positioned at the distal end of the elongated member, the tissue modifier configured to deliver radiofrequency energy for a therapeutic duration; and
a visualization sensor module positioned at the distal end of the elongated member, the visualization sensor module shielded by an enclosure, the enclosure configured to shield the visualization sensor module from a radiofrequency field.

22. The tissue visualization and modification device of claim 21, wherein the visualization sensor module is configured to collect an image of the tissue modifier.

23. The tissue visualization and modification device of claim 21, wherein the tissue modifier comprises an electrode.

24. The tissue visualization and modification device of claim 23, wherein the electrode comprises a metal wire conductor.

25. The tissue visualization and modification device of claim 21, wherein the enclosure comprises a plurality of enclosure elements.

26. The tissue visualization and modification device of claim 21, wherein the visualization sensor module is configured to transmit an image to a display.

27. The tissue visualization and modification device of claim 26, wherein the enclosure is configured to reduce distortion of the image.

28. The tissue visualization and modification device of claim 21, wherein the enclosure comprises a grounded conductive component.

29. The tissue visualization and modification device of claim 21, wherein the enclosure surrounds the visualization sensor module.

30. A tissue visualization and modification device comprising:
an elongated member, extending along a longitudinal axis between a proximal end affixed to a handpiece and a distal end, the distal end comprising a tissue modifier;
a visualization sensor module positioned at the distal end of the elongated member, the visualization sensor module configured to visualize the tissue modifier; and
a light source positioned at the proximal end of the elongated member, the light source coupled to a light conveyance structure, the light conveyance structure configured to convey light to a target tissue.

31. The tissue visualization and modification device of claim 30, wherein the tissue modifier is configured to deliver radiofrequency energy for a therapeutic duration

32. The tissue visualization and modification device of claim 30, wherein the light conveyance structure is an optical fiber.

33. The tissue visualization and modification device of claim 30, wherein the light conveyance structure is contained within a wall of the elongated member.

34. The tissue visualization and modification device of claim 30, wherein the distal end of the elongated member comprises an articulated joint.

35. The tissue visualization and modification device of claim 30, further comprising a prism, the prism positioned distally of the visualization sensor module.

36. The tissue visualization and modification device, further comprising a first polarized member positioned at the distal end of the elongated member, the polarized member configured to reduce the transmission of light from the light source to the visualization sensor module.

37. The tissue visualization and modification device of claim 36, further comprising a second polarized member positioned at the distal end of the elongated member, the second polarized member configured to be complimentary to the first polarized member.

38. The tissue visualization and modification device of claim 30, wherein the tissue visualization module is configured for viewing at an angle between about 45-75 degrees from the longitudinal axis.

39. The tissue visualization and modification device of claim 30, wherein the tissue visualization module is configured for viewing at an angle about 30 degrees from the longitudinal axis.

40. The tissue visualization and modification device of claim 30, further comprising a plurality of illumination elements, the illumination elements positioned concentrically around the visualization sensor module.
